SEQUENCE LISTING (2 sequences)

SEQ ID NO: 1 - protein, 22 aa, synthetic construct
PESKAIKNLLKAVSKERSKRSP

SEQ ID NO: 2 - protein, 16 aa, synthetic construct
KNLLKAVSKERSKRSP

(19) United States
(12) Patent Application Publication
(10) Pub. No.: US 2026/0325657 A1
(43) Pub. Date: Oct. 22, 2026

(54) COMPOSITIONS COMPRISING LINEAR, STAR-SHAPED, AND COMB-LIKE STAPLED P9-PEG CONJUGATES AND METHODS OF USE THEREOF

(71) Applicant: University of Virginia Patent Foundation, Charlottesville, VA (US)

(72) Inventors: Rachel Letteri, Charlottesville, VA (US); Molly A. Hughes, Charlottesville, VA (US); Matthew Crawford, Charlottesville, VA (US); Zixian Cui, Charlottesville, VA (US); Vincent Gray, Charlottesville, VA (US)

(73) Assignee: University of Virginia Patent Foundation, Charlottesville, VA (US)

(21) Appl. No.: 19/099,470

(22) PCT Filed: Jul. 31, 2023
(86) PCT No.: PCT/US2023/029154

§ 371 Date: Jan. 28, 2025

Related U.S. Application Data
(60) Provisional application No. 63/393,755, filed on Jul. 29, 2022.

(51) Int. Cl. A61K 47/60; A61K 38/00; A61P 31/04; C07K 14/47

(52) U.S. Cl. CPC A61K 47/60; A61P 31/04; C07K 14/4723; A61K 38/00

(57) ABSTRACT

Provided are stapled peptides and conjugates thereof that have antibacterial activity against microbial pathogens that include, but are not limited to, multidrug-resistant bacteria. Also provided are stapled peptides and conjugates thereof that are functionalized, encapsulated in a particle, and/or embedded in and/or on a solid support, optionally wherein the stapled peptide and/or conjugate is formulated for release from the solid support, impregnated in a dressing, optionally wherein the stapled peptide and/or conjugate thereof is formulated for release from the dressing, medical devices having a support layer with an antibacterial agent embedded therein or associated therewith, and methods for inhibiting the growth of and/or killing microbes.

### CROSS REFERENCE TO RELATED APPLICATIONS

The presently disclosed subject matter claims priority to and the benefit of U.S. Provisional Patent Application Ser. No. 63/393,755, filed Jul. 29, 2022, the disclosure of which is herein incorporated by reference in its entirety.

### GRANT STATEMENT

This invention was made with government support under Grant Nos. R35 GM147424 and R01 AI150941 awarded by National Institutes of Health. The Government has certain rights in the invention.

### TECHNICAL FIELD

The presently disclosed subject matter relates to compositions and methods useful for treating and preventing bacterial infections. In particular, the presently disclosed subject matter relates to bactericidal peptide-based compositions that can be administered to subjects to treat and/or prevent microbial infections, undesirable cellular proliferation, and diseases, disorders, and conditions related thereto.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter relates in some embodiments to stapled peptides based on the amino acid sequence PESKAIKNLLKAVSKERSKRSP (SEQ ID NO: 1) or a truncated version thereof. In some embodiments, the stapled peptides comprise, consist essentially of, or consist of two modifications of the amino acid sequence PESKAIKNLLKAVSKERSKRSP (SEQ ID NO: 1) or the truncated version thereof, wherein the two modifications are designed to provide stapling sites for the peptide. In some embodiments, the two modifications comprise, consist essentially of, or consist of substitutions of two amino acids of SEQ ID NO: 1 that are separated by three intervening amino acids, wherein the modifications replace the amino acid present at a given location with 2-(4-pentenyl) alanines or other appropriate amino acid derivative. In some embodiments, the two modifications comprise, consist essentially of, or consist of substitutions of two amino acids of SEQ ID NO: 1 that are separated by six intervening amino acids, wherein the modifications replace the amino acid present at a given location with 2-(4-octenyl) alanine or other appropriate amino acid derivative. In some embodiments, the peptides containing the two modifications are stapled. In some embodiments, the two modifications are substitutions of amino acids 8 and 12 of SEQ ID NO: 1, or corresponding positions of a modified, optionally truncated, peptide, with 2-(4-pentenyl) alanines and/or the two modifications are substitutions of amino acids 5 and 12 of SEQ ID NO: 1, or corresponding positions of a modified, optionally truncated, peptide, with 2-(4-octenyl) alanines. In some embodiments, the stapled peptide is a truncated version of SEQ ID NO: 1, which in some embodiments comprises, consists essentially of, or consists of the amino acid sequence KNLLKAVSKERSKRSP (SEQ ID NO: 2).

In some embodiments, a stapled peptide of the presently disclosed subject matter further comprises a linking moiety at the N-terminus of the stapled peptide, the C-terminus of the stapled peptide, or both, wherein the linking moiety is designed for linking the stapled peptide to a polymer. In some embodiments, the linking moiety is an Ac-CGG moiety, a methacrylamide-(CH₂)₆ moiety, or a combination thereof.

In some embodiments of the presently disclosed subject matter, the stapled peptide is relatively more resistant to protease cleavage than a peptide with the same amino acid sequence that is not stapled. In some embodiments, the enhanced protease cleavage resistance results from one or more, optionally all, of the amino acids of the stapled peptide being D-amino acids.

In some embodiments, the presently disclosed subject matter also relates to truncated versions of the presently disclosed stapled peptides. In some embodiments, the truncated version comprises, consists essentially of, or consists of the amino acid sequence KNLLKAVSKERSKRSP (SEQ ID NO: 2).

In some embodiments, the presently disclosed subject matter relates to stapled peptides that include one or more amino acid substitutions relative to SEQ ID NO: 1 or 2. Exemplary amino acid substituted derivatives of P9 that exhibit antimicrobial activity and that can therefore be employed in the compositions of the presently disclosed subject matter are disclosed in PCT International Patent Application Publication No. WO 2020/023670 (incorporated herein by reference in its entirety), including particularly in FIG. 18. In some embodiments, the substitutions of the two amino acids in the stapled peptide of the presently disclosed subject matter or the truncated version thereof do not involve any cationic amino acids (in some embodiments, occur at amino acids other than arginine or lysine).

The presently disclosed subject matter also relates in some embodiments to conjugates comprising a stapled peptide of the presently disclosed subject matter conjugated to one or more polymers. In some embodiments, the one or more polymers are selected from the group consisting of a poly(ethylene glycol) (PEG) molecule, a zwitterionic polymer, a poly(hydroxypropyl methacrylamide) polymer, or any combination thereof. In some embodiments, the conjugate is a linear conjugate, a comb-shaped conjugate, a bottlebrush-shaped conjugate, and/or a star-shaped conjugate. In some embodiments, the star-shaped conjugate has 3, 4, 5, 6, 7, or 8 arms. In some embodiments, each arm individually comprises a PEG group of 1-20 kDa. In some embodiments, the conjugate forms a nanostructure, optionally a micelle, of about 10-20 nm diameter in water.

The presently disclosed subject matter also relates in some embodiments to comb-shaped conjugates comprising a stapled peptide of the presently disclosed subject matter and/or a truncated stapled peptide thereof, wherein at least one stapled peptide is modified with a polymerizable group, optionally a methacrylamide, copolymerized with a hydrophilic vinyl monomer, optionally poly(ethylene glycol) methacrylate, a 2-hydroxypropylmethacrylamide, a zwitterionic methacrylate, or any combination thereof. In some embodiments, the comb-shaped conjugate comprises a density of peptide monomers on the comb ranging from 1% to 100%. In some embodiments, the comb-shaped conjugate comprises 4, 8, or 16 peptides. In some embodiments, the molecular weight of the comb-shaped conjugate is about 50-70 kDa.

The presently disclosed subject matter also relates in some embodiments to comb-shaped conjugates with a peptide attached at its N- and/or the C-terminus and/or at an intermediate amino acid. In some embodiments, the internal amino acid is a lysine comprising an amine-functionalized side chain.

In some embodiments of the presently disclosed conjugates and/or comb-shaped conjugates, the conjugate and/or the comb-shaped conjugate form a nanoassembly, in some embodiments a micelle, of about 10-20 nm diameter in water.

The presently disclosed subject matter also relates in some embodiments to physical mixtures and/or formulations comprising at least one stapled peptide as disclosed herein and one or more polymers that complex the at least one stapled peptide via an electrostatic interaction. In some embodiments, the physical mixture comprises poly(methacrylic acid) and the at least one stapled peptide of the presently disclosed subject matter. In some embodiments, the physical mixture and/or formulation comprises at least one polymer that complexes the at least one stapled peptide via an stereochemistry-driven interaction. In some embodiments, the physical mixture comprises a mixture of conjugates of polymers and D-peptide versions of the at least one stapled peptide, wherein a D-peptide version of the at least one stapled peptide is capable of forming a complex with an L-peptide version of the at least one stapled peptide. In some embodiments, the physical mixture and/or formulation comprises a D-stapled peptide complexed to one or more conjugates of polymers, L-stapled peptides, or both.

The presently disclosed subject matter also relates in some embodiments to pharmaceutical compositions comprising, consisting essentially of, or consisting of a stapled peptide of the presently disclosed subject matter, a truncated version thereof, a conjugate of the presently disclosed subject matter, a comb-shaped conjugate of the presently disclosed subject matter, or any combination thereof, and a pharmaceutically acceptable carrier, diluent, and/or excipient. In some embodiments, the pharmaceutical composition is pharmaceutically acceptable for use in a human.

The presently disclosed subject matter also relates in some embodiments to medical devices comprising a support layer with an antibacterial agent embedded therein or associated therewith, wherein the antibacterial agent comprises a stapled peptide as disclosed herein, a truncated version thereof, a conjugate as disclosed herein, a comb-shaped conjugate as disclosed herein, or any combination thereof. In some embodiments, the medical device is a wound dressing. In some embodiments, the stapled peptide, truncated version thereof, conjugate, or comb-shaped conjugate, or any combination thereof, is encapsulated in a particle that is embedded in or associated with the support layer.

The presently disclosed subject matter also relates in some embodiments to methods for inhibiting growth of and/or for killing a bacterium. In some embodiments, the methods comprise, consist essentially of, or consist of contacting the bacterium with an effective amount of an antibacterial agent selected from the group consisting of a stapled peptide of the presently disclosed subject matter, a truncated version thereof, a conjugate of the presently disclosed subject matter, a comb-shaped conjugate of the presently disclosed subject matter, or any combination thereof. In some embodiments, the bacterium is selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to a Salmonella enterica serovars such as but not limited to Salmonella enterica serovar Typhi and Shigella flexneri; and biothreat agents such as but not limited to Bacillus anthracis in both vegetative and spore forms.

The presently disclosed subject matter also relates in some embodiments to methods for treating bacterial infections present in wounds. In some embodiments, the methods comprise, consist essentially of, or consist of contacting the wound with an effective amount of a composition comprising a stapled peptide of the presently disclosed subject matter, a truncated version thereof, a conjugate of the presently disclosed subject matter, a comb-shaped conjugate of the presently disclosed subject matter, or any combination thereof.

The presently disclosed subject matter also relates in some embodiments to uses of the presently disclosed stapled peptides, truncated versions thereof, conjugates thereof, comb-shaped conjugates thereof, or any combination thereof for preventing or treating a bacterial infection.

The presently disclosed subject matter also relates in some embodiments to compositions for use in preventing and/or treating bacterial infections. In some embodiments, the compositions comprise, consist essentially of, or consist of a stapled peptide of the presently disclosed subject matter, a truncated version thereof, a conjugate thereof, a comb-shaped conjugate thereof, or any combination thereof. In some embodiments, the compositions for use of the presently disclosed subject matter further comprise a pharmaceutically acceptable carrier, diluent, and/or excipient. In some embodiments, the composition is pharmaceutically acceptable for use in a human.

Accordingly, it is an object of the presently disclosed subject matter to provide stapled peptides, truncated versions thereof, conjugates thereof, and combination thereof, and methods for their uses.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following Detailed Description and Figures.

### BRIEF DESCRIPTION OF THE FIGURES

FIGS. 1A and 1B. Modulating presentation of therapeutic peptides within polymers by varying architecture (FIG. 1A) and supramolecular assembly (FIG. 1B) to control biophysical properties relevant to therapeutic performance.

FIGS. 2A and 2B. Synthesis of comb-shaped polymer-peptide conjugates using controlled radical polymerization to independently tune peptide density and polymer chain length.

FIG. 3. Stapled P9-PEG conjugation scheme, in which the thiol-terminated AMP stapled Ac-CGGP9 was appended to maleimide-functionalized linear and star-shaped PEG, using 1.1 or 1.25 equivalents of peptide relative to maleimides on linear and star-shaped polymer, respectively. Following dialysis to remove unreacted peptide, NMR showed the linear, 4-arm, 8-arm, and short 8-arm polymers to have 1, 2.8, 7.4, and 7.3 peptides arms, respectively. A P9 variant (differs from P9 by the acetyl-cysteine-glycine-glycine linker at the N-terminus and the hydrocarbon staple in the middle) is shown. The cysteine thiol at the N-terminus (highlighted in gray) was employed to attach the peptide to maleimide-decorated star polymers with varying arm lengths and numbers. The maleimide groups on the polymers are also circled in gray.

FIGS. 4A-4C. Circular dichroism data showing the stapled P9 peptide retained its helical structure after conjugation to polymers in a variety of environments: phosphate buffer (PB; FIG. 4A), PB containing trifluoroethanol (helix-promoting; PB-TFE; FIG. 4B), and PB containing SDS (bacterial membrane-mimicking detergent; PB-SDS; FIG. 4C).

FIG. 5. Summary of the zeta potentials (proportional to surface charge), sizes (in solution), antimicrobial activities, and hemolytic activities of the stapled P9 variant and exemplary conjugates. Conjugation to neutral hydrophilic polymers decreased zeta potential (surface charge). Linear conjugates had the lowest zeta potential-which, taken together with the large size in solution-suggested that these linear conjugates might assemble into micelles in solution with a peptide core surrounded by a neutral polymer shell. Antimicrobial activity increased with peptide content. Even if peptide was surrounded by polymer in the core of a micelle, and the linear conjugates indeed assembled in solution, the structures appeared to still have antimicrobial activity. None of the formulations caused hemolysis.

FIG. 6. Transmission electron microscopy (TEM) images showing the linear conjugates formed 20 nm micelles—a smaller size than in solution—but this was normal due to drying effects. These peptide-polymer conjugates had the ability to assemble into supramolecular (multi-molecular) structures in solution, which can provide advantages. There were definitely existing antimicrobial peptide-polymer micelles. Another of the conjugates also seemed to assemble in solution, and since the solution-state size measurements seemed to show 5 nm structures it was possible that the this was operating close to a critical concentration (below which the supramolecular micelles can disassemble and dissolve).

FIG. 7. A second group of exemplary conjugates also showed the linear conjugates to have the lowest zeta potential (surface charge), but this time assembly into micelles was not observed. However, since the polymer was neutral and hydrophilic and the peptide was cationic and had some hydrophobic character, it is possible that it can form supramolecular micelles and that the micelles could have antimicrobial activity and mechanisms of action that are distinct from single (unassembled) micelles.

FIG. 8 summarizes activity on a mass and mole-of-peptide basis of formulations from two batches of materials.

FIG. 9. The polymers partially shielded the peptides from proteolytic degradation. The top left gray high-performance liquid chromatography (HPLC) traces showed the peptide alone (without a staple) before (top) and after 1 hour (bottom) of incubating the peptide with Proteinase K, which likely cleaved the peptide in all of the locations shown by red arrows. After 1 hour, the peak at around 5 minutes disappeared completely, indicating no more intact peptide. The peaks eluting earlier that appeared after 1 hour were likely fragments of the peptide. The stapled peptide (black trace-top middle) also did not remain intact after 1 hour—the peak shifted to slightly earlier positions-suggesting that it may break only in one or two places—the staple may provide some protection against degradation. All conjugation strategies, however, appeared to protect the stapled peptide—the original peak just before 6 minutes remained even after 1 hour in all of the peptide-polymer formulations. There is some degradation (new peaks appeared), but some of the peptide/original conjugate remained intact.

FIG. 10. This graph summarizes the data of FIG. 9 and adds time points, showing how much of the original peak (for either the peptide or conjugate) remains intact after 30 minutes, 1 hour, 2 hours, and 20 hours incubation with Proteinase K. 25 FIG. 11. In addition to star-shaped polymers, P9 and stapled P9 were also modified so that they could be incorporated into comb/bottlebrush-shaped polymers. Here is shown peptide P9 modified at the N-terminus with a polymerizable methacrylamide group separated from the N-terminal proline residue by a 6-carbon linker. The polymerizable peptide is referred to as mAhxP9. Reversible addition fragmentation chain transfer (RAFT) polymerization—a metal-free controlled polymerization technique—was used to generate copolymers of a neutral hydrophilic monomer (2-hydroxypropyl methacrylamide), but this can also be accomplished with poly(ethylene glycol) methacrylates and other neutral hydrophilic monomers like zwitterionic sulfobetaine methacrylate and methacryloyloxyethyl phosphoryl choline. This technique can be employed to vary the number and density of peptides along the polymer chain and the total molecular weight.

FIG. 12. These are size exclusion chromatography results showing that mAhxP9 was co-polymerized with the neutral hydrophilic monomer HPMA. The top plot shows that after blending the monomers in a 1:9 ratio, about half of the peptide monomer reacted and all the small HPMA reacted. Suspecting the small monomer reacts faster than the large one, but that the large peptide monomer needs to be spaced out by smaller monomer (e.g., to avoid crowding), the small monomer was added in 2 batches with the second batch being added after 24 hours. This appeared to result in incorporation of more of the peptide to make a larger polymer, as seen by the larger (eluting at earlier times) comb polymer peak. Note that since the monomer was added half-way through, and therein diluted the mixture, peak intensity cannot be related to amount consumed on the bottom plot.

FIG. 13. Comb polymers with poly(ethylene glycol) methacrylate as the neutral hydrophilic monomer were also prepared. Data for the comb polymers are shown in FIGS. 14 and 15.

FIG. 14. Top set of plot shows that the two co-polymerized, but the small monomer incorporated/reacted much faster than the large peptide.

FIG. 15. This is a replicate of the experiment presented in FIG. 14 but with purified (>95% pure) peptide monomers.

FIG. 16. Engineering molecular architectures of AMP-PEG conjugates to balance antimicrobial activity, cytotoxicity, and proteolytic stability. Free AMPs (orange) exhibit high bactericidal activity but are not proteolytically stable and can be toxic to mammalian cells. Conjugating AMPs to long linear PEG chains (on the right side of the balance) will often decrease the toxicity and improve stability, but also significantly reduce antimicrobial activity. Since shortening PEG usually results in conjugates approaching the behavior of free peptide, we are sought to design star-shaped AMP-PEG conjugates with various arm number and arm lengths to better balance this trade-off.

FIGS. 17A-17D. SEC traces of stapled Ac-CGGP9 (black), PEG maleimide (lighter gray) with different architectures, and corresponding conjugates (darker gray). FIG. 17A) linear; FIG. 17B) 4-arm star; FIG. 17C) 8-arm star; FIG. 17D) short 8-arm star.

FIGS. 18A-18C. AlamarBlue assay to test activity of peptide/conjugates against K. pneumoniae. FIG. 18A) mechanism of alamarBlue reagent: the blue dye molecule can be reduced in pink and fluorescent in living bacteria. FIG. 18B) example 96-well plate with colors from pink, purple, to blue, suggesting the amounts of bacteria from high, medium, to low. FIG. 18C) Bacteria survival after treated by peptide/conjugate at 100 μM peptide in RPMI. Three batches of materials (square, triangle, and circle) were tested in triplicate. Results are plotted for each batch and average. Error bars represent the standard deviation of 9 data points.

FIGS. 19A and 19B. Proteolytic stability of stapled P9-PEG against Proteinase K. FIG. 19A) possible cleavage sites on stapled Ac-P9 (predicted by https://web.expasy.org/peptide_cutter/); FIG. 19B) summary of retained peptide fraction treated by Proteinase K after 0.5, 1, 2, and 24 h. Fractions were calculated from the peak area ratio from HPLC traces (see Table 2). Peak area values were normalized by peptide/conjugate without treatment.

FIGS. 20A-20C. Zeta potential, size, and morphology of stapled Ac-P9 and its conjugates. FIG. 21A) Zeta potential (circles) and size (bars) of stapled Ac-P9 and its conjugates at 200 M peptide in 10 mM PBS. The diameters were reported as the Z-average diameter from DLS (Dh, DLS) and average size measured from TEM images (DTEM). Error bars represent the standard deviation of triplicate measurements. Peptide content was calculated as the weight percent peptide of the molecules. FIG. 21B) Representative TEM images of stapled Ac-P9 and its conjugates at 200 μM peptide in 10 mM PBS, showing more distinct assemblies of the linear and short 8-arm conjugates than from the other conjugates. Scale bar=50 nm. FIG. 21C) Proposed schemes of assemblies of stapled Ac-P9 and its conjugates. Stapled Ac-P9 peptides aggregate randomly, while the linear conjugates form micelles with PEG chain on the outside, just partially hide the peptide content due to the similar hydrophobicity between PEG and peptide. With the low peptide content, the 4-arm and 8-arm conjugates may form ‘flower-shape’ structures with PEG shells and peptide cores. For the 4-arm conjugate with ca. 3 peptide arms on one molecule, the free PEG arms possibly make the PEG shells to be ‘loose’ and provide opportunity for peptide arms to be on the outside. For the 8-arm conjugates, the high arm number may constrain the assemblies of the conjugates into structures with defined cores and shells. The short 8-arm conjugates may form micelles that having the short PEG arms bridging the peptide cores instead of forming shells completely protecting the peptide content.

FIG. 21. Stapling of P9 via metathesis reaction.

FIGS. 22A-22C. RAFT copolymerization of mAhxP9 with HPMA into comb polymers. FIG. 22A) reaction scheme of the polymerization. FIG. 22B) When all monomer is added at once, HPMA reaches a higher conversion than peptide monomer. FIG. 22C) By adding HPMA in two batches, the copolymer shows a slightly higher molecular weight.

FIG. 23. Conjugation of GGGL8 to linear and star-shaped PEG NHS. While attaching GGGL8 to linear PEG NHS yielded side reactions. We developed a protection-conjugation-deprotection strategy to protect the thiol groups during conjugation reaction and remove the protecting groups after conjugation.

FIG. 24. Synthesis of 8-arm L8-PEG conjugate in which L8 peptides are attached to PEG at the C-terminus.

FIGS. 25A-25D. Stapled Ac-P9: FIG. 25A) structure; FIG. 25B) analytical HPLC traces of crude and purified stapled Ac-P9; FIG. 25C) analytical HPLC traces crude and purified stapled Ac-P9 with a shallower eluent gradient; FIG. 25D) MALDI-TOF MS of purified stapled Ac-P9, [M+Na]+: calculated m/z=2550.6+23.0=2573.6 g/mol; found 2573.0 g/mol.

FIGS. 26A-26E. S2 Stapled Ac-CGGP9: FIG. 26A) structure; FIG. 26B) analytical HPLC traces of crude and purified stapled Ac-CGGP9; FIG. 26C) analytical HPLC traces crude and purified stapled Ac-CGGP9 with a shallower eluent gradient; FIG. 26D) preparative HPLC trace, highlighting collected fractions; FIG. 26E) MALDI-TOF MS of purified stapled Ac-CGGP9, [M+Na]+: calculated m/z=2765.6+23.0=2788.6 g/mol; found 2789.3 g/mol.

FIGS. 27A-27D. HPLC traces of purified stapled P9, PEG maleimide, conjugate mixtures after reacting 1 and 3 h, and purified conjugates. FIG. 27A) linear; FIG. 27B) 4-arm star; FIG. 27C) 8-arm star; FIG. 27D) short 8-arm star. After dialysis all conjugates were >95% pure (contained <5% unreacted peptide).

FIG. 28. ¹H NMR (800 MHz, D₂O) spectrum of stapled Ac-CGGP9. Peaks a and b contain the alkene protons on the staple, and these signals integrate approximately to the expected 2H per peptide. Peak c and d, overlapped with the peaks of 25 αHs on all amino acids, contain 4 Pro δH and 8 Ser δH, respectively. Peak e contains 4 Arg δH. Peaks f and g contains 2 Cys βH and 10 Lys εH, respectively. The remaining 113 protons on the peptide side chains appear between 0.7-2.6 ppm.

FIGS. 29A and 29B. ¹H NMR (800 MHz, D₂O) spectrum of FIG. 29A) linear PEG maleimide and FIG. 29B) linear stapled P9-PEG conjugate.

FIGS. 30A and 30B. ¹H NMR (800 MHz, D₂O) spectrum of FIG. 30A) 4-arm PEG maleimide and FIG. 30B) 4-arm stapled P9-PEG conjugate, showing the conjugates to contain an average of 2.8 peptide arms.

FIGS. 31A and 31B. ¹H NMR (800 MHz, D₂O) spectrum of FIG. 31A) 8-arm PEG maleimide and FIG. 31B) 8-arm stapled P9-PEG conjugate, showing the conjugates to contain an average of 7.4 peptide arms.

FIGS. 32A and 32B. ¹H NMR (800 MHz, D₂O) spectrum of FIG. 32A) short 8-arm PEG maleimide and FIG. 32B) short 8-arm stapled P9-PEG conjugate, showing the conjugates to contain an average of 7.3 peptide arms.

FIG. 33. Metabolic activity of Klebsiella Pneumoniae after treating with peptide/conjugates at 100 or 200 μM peptide. Bacteria and peptide/conjugates were incubated at 37° C. for 2 h before adding alamarBlue agent. The average fluorescence generated by metabolically active bacteria was read after 2-3 h and was normalized by the fluorescence generated by untreated bacteria. Each batch was tested in triplicate, and error bars represent the standard deviation of the repeats.

FIG. 34. Hemolysis. Human red blood cells were exposed to stapled Ac-P9 peptides/conjugates at 200 μM peptide, water, or 10 μM melittin in RPMI buffer with 1% BSA for 1 h. Hemolysis % was normalized by the color of red blood cells treated by 1% Triton X-100, and reported as the average from n=3-4 donors, with error bars representing the standard deviation. ** p<0.001 and *p<0.05 as compared to the vehicle-alone control by one-way ANOVVA with Dunnett's multiple comparisons test, ns=not significant.

FIG. 35. HPLC traces of stapled Ac-P9 incubated with Proteinase K after 0.5, 1, 2, and 24 h in RPMI buffer. More stapled Ac-P9 was added to the mixture to test the functionality of Proteinase K. The peptide peak increased initially due to the spike, and then decreased 40 min after spiking the sample, showcasing the Proteinase K was still active after 24 h and can be used to monitor degradation of conjugates in 24 h. Highlighted peaks corresponding to intact peptide were used for calculation of the remained peptide fraction.

FIG. 36. HPLC traces of linear stapled P9-PEG conjugate incubated with Proteinase K after 0.5, 1, 2, and 24 h in RPMI buffer at room temperature. Highlighted peaks were used for calculation.

FIG. 37. HPLC traces of 4-arm stapled P9-PEG conjugate incubated with Proteinase K after 0.5, 1, 2, and 24 h in RPMI buffer. At room temperature Highlighted peaks were used for calculation.

FIG. 38. HPLC traces of 8-arm stapled P9-PEG conjugate incubated with Proteinase K after 0.5, 1, 2, and 24 h in RPMI buffer at room temperature. Highlighted peaks were used for calculation.

FIG. 39. HPLC traces of short 8-arm stapled P9-PEG conjugate incubated with Proteinase K after 0.5, 1, 2, and 24 h in RPMI buffer at room temperature. Highlighted peaks were used for calculation.

FIGS. 40A and 40B. Summary of retained peptide fraction treated by Proteinase K after 0.5, 1, 2, and 24 h. Fractions were calculated from the peak area ratio from HPLC traces (Table 2). Peak area values were normalized by peptide/conjugate without treatment. FIGS. 40A and 40B are two runs with two batches of materials synthesized independently.

FIG. 41. HPLC traces of stapled Ac-CGGP9 with treatment of Proteinase K in 1×PBS after 0.5, 1, 2, and 24 h.

FIG. 42. HPLC traces of linear stapled P9-PEG conjugate incubated with Proteinase K after 0.5, 1, 2, and 24 h in 1×PBS at room temperature. Highlighted peaks were used for calculation.

FIG. 43. HPLC traces of 4-arm stapled P9-PEG conjugate incubated with Proteinase K after 0.5, 1, 2, and 24 h in 1×PBS at room temperature. Highlighted peaks were used for calculation.

FIG. 44. HPLC traces of 8-arm stapled P9-PEG conjugate incubated with Proteinase K after 0.5, 1, 2, and 24 h in 1×PBS at room temperature. Highlighted peaks were used for calculation.

FIG. 45. HPLC traces of short 8-arm stapled P9-PEG conjugate incubated with Proteinase K after 0.5, 1, 2, and 24 h in 1×PBS at room temperature. Highlighted peaks were used for calculation.

FIGS. 46A-46C. CD spectra of stapled Ac-CGGP9 and the corresponding conjugates in different solvents with mass concentration of 0.1 mg/mL at 25° C.: FIG. 46A) 10 mM phosphate buffer (PB); FIG. 46B) 50% v/v TFE in 10 mM PB; and FIG. 46C) 60 mM SDS in 10 mM PBS. Measurements were performed in triplicate, and the data are plotted as an average of the three runs.

FIG. 47. Correlograms of peptide/conjugates at concentrations of 400 (left), 200 (middle), and 100 (right) μM peptide in 10 mM PBS. Measurements were performed in triplicate.

FIGS. 48A-48D. Intensity profiles of stapled Ac-P9-PEG conjugates at 100, 200, and 400 μM peptide in 10 mM PBS: FIG. 48A) linear conjugate; FIG. 48B) 4-arm conjugate; FIG. 48C) 8-arm conjugate; and FIG. 48D) short 8-arm conjugate. The darkness of colors indicates different peptide concentrations.

FIG. 49. TEM images and histograms of stapled Ac-P9 and its conjugates at 200 μM peptide in 10 mM PBS. TEM images were taken at 200 μM peptide concentration and were analyzed by ImageJ and the histograms were obtained from Excel by counting 200 samples. Scale bar=100 nm.

FIGS. 50A-50H. Representative TEM images of stapled Ac-P9 and its conjugates at 200 μM peptide in 10 mM PBS: FIG. 50A) 10 mM PBS; FIG. 50B) stapled Ac-P9; FIG. 50C) linear PEG maleimide; FIG. 50D) linear conjugates; FIG. 50E) short 8-arm PEG maleimide; FIG. 50F) short 8-arm conjugate; FIG. 50G) 4-arm conjugate; and FIG. 50H) 8-arm conjugate. The polymer control samples were prepared at same mass concentration of polymer content on corresponding conjugate. Scale bar=100 nm.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. General Considerations

Overuse or misuse of antibiotics leads bacteria to develop resistance, which has been predicted to cause infections that result in 10 million deaths globally and cost trillions of dollars annually by 2050. Therefore, novel antimicrobial agents are needed to treat infections and lower associated costs. As one promising class of therapeutics, cationic and helical antimicrobial peptides (AMPs) can induce membrane disruption via hydrophobic and electrostatic interactions with bacterial membrane, while invoking less and slower resistance than conventional antibiotics. However, the clinical implementation and application of AMPs are limited by their rapid clearance, proteolytic instability, and toxicity to mammalian cells. According to a 2022 report on the Data Repository of Antimicrobial Peptides (DRAMP), only 0.3% enter clinical trials and only one is FDA approved for human immunodeficiency virus (HIV) treatment.

To overcome the aforementioned challenges, different delivery strategies for administering AMPs have been studied, including physical encapsulation and chemical conjugation, with carriers ranging from inorganic (e.g., gold, silver, and silica-based nanoparticles) to organic (e.g., polymer, micelle, liposome, and hydrogel) compositions. In general, such vehicles improve solubility of AMPs, shield toxic interactions with healthy cells, slow enzymatic degradation of peptide constituents by blocking protease access, and yield larger, stealthy molecules that evade rapid renal or immune system-mediated clearance. Among potential delivering strategies, attaching AMPs to polymers allows control of carrier composition and architecture, providing unique opportunities to enhance stability and lower toxicity while retaining antimicrobial activity.

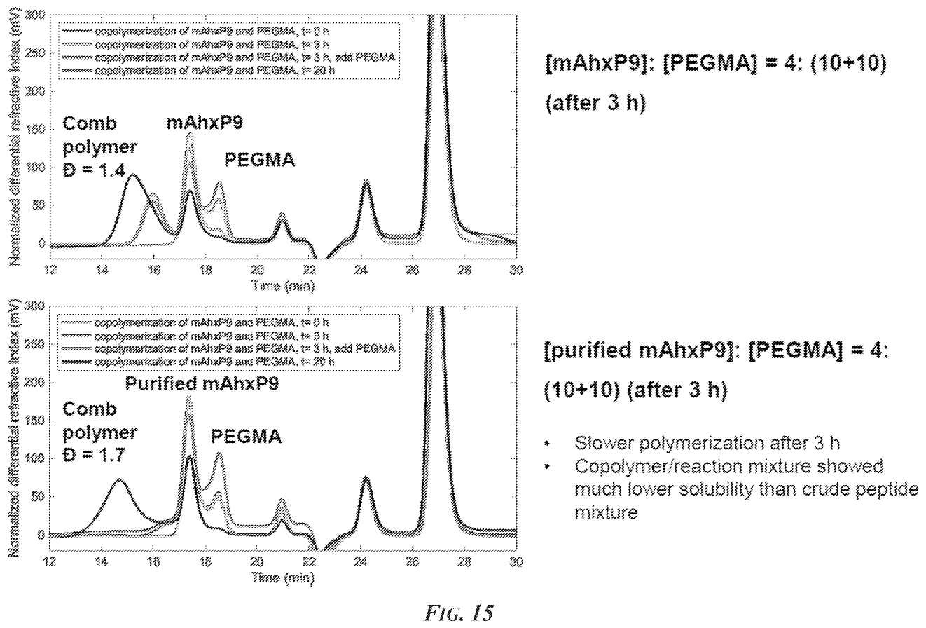

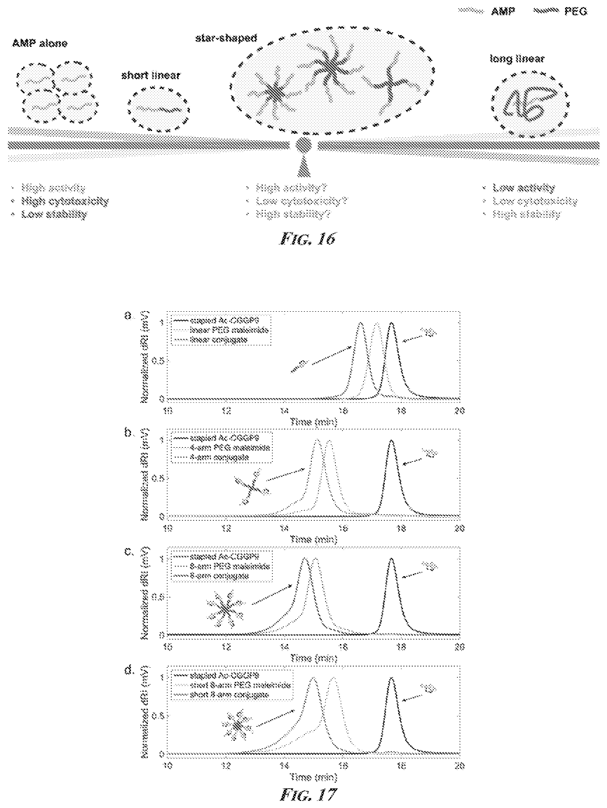

As the most straightforward conjugation scheme, AMPs can be conjugated to the end of a long polymer chain. For example, linear conjugates of AMPs and neutral hydrophilic poly(ethylene glycol) (PEG) prevent proteolytic degradation and toxic interactions of the AMPs with mammalian cells due steric hinderance provided by the long polymer chain, but also mask the peptide from attacking bacterial membranes. Attaching AMPs to shorter PEG chains improves activity, but often at the expense of faster degradation and compatibility with mammalian cells. Thus, simply varying the chain length of polymer conjugated to AMPs results in a dilemma of either wrapping the AMP and preventing it from attacking bacteria or leaving it more exposed on polymer facing similar limitations of the free peptide. (see FIG. 16).

Advances in polymer chemistry allow the preparation of AMP-polymer conjugates with a variety of architectures (e.g., star-shaped, comb-like, and hyperbranched). With multiple AMPs attached to one molecule, non-linear architectures provide opportunities to better balance activity, stability, and toxicity compared to linear analogs. Star-shaped polymers, having multiple arms emanating from a core, allow multivalency of AMPs on the molecule and variations in both arm number and arm length to change peptide content on molecules to improve their antimicrobial activity. For example, conjugates having multiple poly(lysine-co-valine) AMP arms attached to a cationic poly(amido amine) (PAMAM) core exhibit superior antibacterial activity relative to the poly(lysine-co-valine) AMP alone. Moreover, increasing the number or length of AMP arms on the star-shaped conjugates enhanced activity, presumably due to the higher density and molecular weight of the AMPs. Star-shaped poly(lysine)-PEI conjugates also showed higher proteolytic stability than linear poly(lysine) AMP alone with a similar molecular weight. However, these conjugates with cationic polymer cores still suffer from the trade-off between antimicrobial activity and toxicity. Conjugates with cationic PAMAM cores improved activity again E. coli (MIC from 8 to 5 μg/mL, calculated based on molecular weight×conjugate concentration) but decreased mammalian cell viability (IC₅₀ from 63 to 32 μg/mL).

While neutral hydrophilic polymers in linear conjugates impart stability and mammalian cell compatibility at the expense of antimicrobial activity, they are very attractive constituents of non-linear conjugates. For example, star-shaped AMP-polymer conjugates have been prepared with a mixture of neutral glucosamine polymer and cationic AMP (poly(lysine)) arms. Encouragingly, increasing the fraction of peptide arms increased activity against MRSA (MIC from 128 to 32 μg/mL) while only very slightly decreasing cell viability (IC₅₀ from 369 to 337 μg/mL). However, few other studies involved neutral hydrophilic polymers.

As combining AMPs with neutral hydrophilic polymers in non-linear conjugates is promising, it is important to understand learn their solution behavior to connect molecular architecture to antimicrobial performance. Since AMPs are often cationic and hydrophobic, combining them with neutral hydrophilic PEG may induce phase separation and supramolecular assembly. Indeed, several linear PEGylated AMPs assemble into micelles or nanoparticles with peptide cores and PEG shells. In these cases, rather than the polymer wrapping the AMP and reducing antimicrobial activity, the resulting supramolecular structures show enhanced activity attributed to the multivalency of AMPs. Moreover, these supramolecular structures exhibit the lower toxicity to mammalian cells and higher stability imparted by the shielding effects of PEG. Similar to linear conjugates, the star-shaped conjugates may form supramolecular assemblies, either with AMP cores surrounded by PEG shells or vice versa in aqueous solution. Therefore, it is essential to understand if star-shaped AMP-PEG conjugates with various arm numbers and arm lengths assemble in solution and therein modulate how AMPs are presented and change the morphology and properties in solution, and by extension the antimicrobial performance.

As disclosed herein, a series of AMP-PEG conjugates were prepared, varying arm number and arm length, to investigate the architectural effects on their solution properties and performance (i.e., antimicrobial activity, proteolytic stability, and hemolysis). A sequence derived from CXCL10, stapled P9, which showed high activity against multiple bacterial strains without introducing significant hemolysis at 50 μM was selected. As this helical stapled P9 is similar to many typical membrane-disrupting AMPs in cationic, hydrophobic and helical character, it was chosen as the model peptide and envision the design rules developed here can be extended to guide the molecular design of other AMP-polymer conjugates. To understand how architecture affects the presentation of peptide on conjugates in solution, the performance results to some of the performance-determining properties (i.e., secondary structure, size, zeta potential, and morphology) were studied. The findings and methods described herein can guide the molecular design of AMP-polymer conjugates in the future.

Peptides hold tremendous promise as therapeutics. They capably eradicate infections, kill cancer cells, control immune system responses, and provide restorative and/or protective effects to a wide range of cells with few off-target effects and few if any toxic metabolites. Yet, degradation by proteases, removal of small molecular structures <20 nm from the blood stream through renal filters, and immune system-mediated clearance result in undesirably short half-lives of peptides in the body and hinder clinical implementation.

Furthermore, peptides that combat infectious microorganisms or cancer cells can exhibit undesired cytotoxic effects. Combining peptides with larger polymer structures that not only increase size in solution to avoid renal filtration, but also shield peptides from undesired destabilizing interactions with proteases, immune system components, and healthy cells, offers a compelling, comprehensive solution to these limitations (Kopecek & Yang, 2020).

Both conjugation of peptides to polymers and encapsulation of peptides within polymeric particles can modulate the intended interactions of therapeutic peptides, and by extension promote therapeutic efficacy; however, this requires careful consideration of peptide-polymer conjugate or formulation structure, composition, and properties. Perhaps the best example of the benefit of conjugating therapeutics to polymers is the attachment of the protein interferon 2α to the ends of a star-shaped biocompatible polymer to extend its lifetime in the body (Joralemon et al., 2010). Known as PEG-INTRON™, these conjugates now treat cancer, hepatitis, and other conditions. Additionally, attaching multiple antimicrobial peptides to star, comb, or hyperbranched polymers increases antimicrobial activity by increasing the local concentration of peptide, however in some cases also results in toxic interactions with mammalian cells (Cui et al., 2021). On the other hand, attaching an antimicrobial peptide to one end of a charge-neutral, biocompatible polymer decreases toxicity to mammalian cells, but also markedly reduces antimicrobial activity (Imura et al., 2007). The critical challenge—and immense opportunity for us working at the intersection of polymers and medicine—is in engineering the presentation of peptides within polymer scaffolds to bolster the efficacy of peptide therapeutics, rather than hampering target effects or causing deleterious side effects.

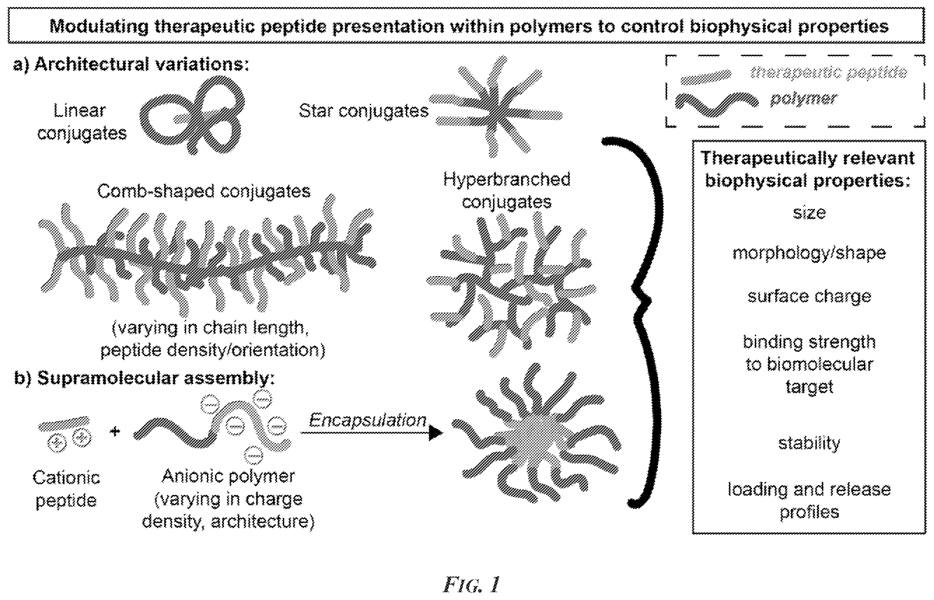

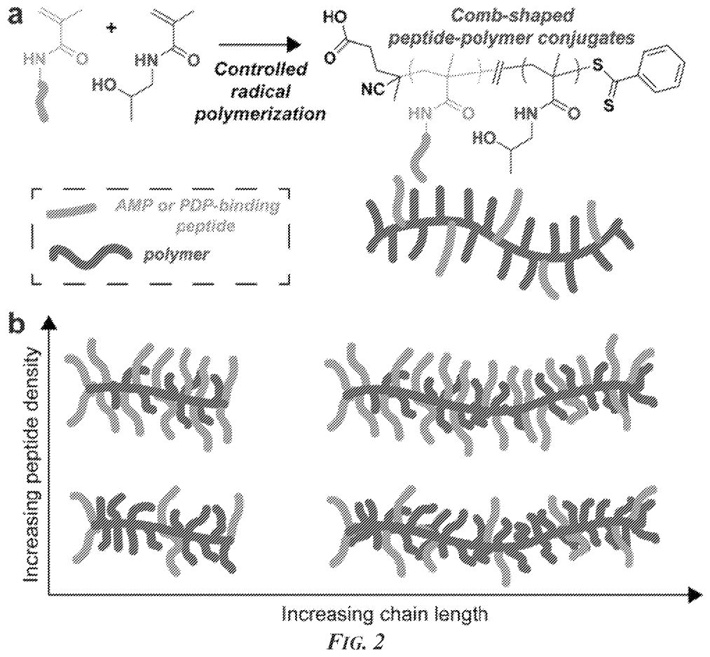

Advances in synthetic methods for preparing polymers and peptide-polymer conjugates continue to unlock unprecedented opportunities to control peptide density, polymer composition, architecture (e.g., linear vs. comb), molecular weight, and supramolecular assembly (see FIG. 1). Previous work by the present co-inventors (Parelkar et al., 2014; Ghobadi et al., 2016) together with the work of others (Johnson et al., 2010; Johnson et al., 2011; Blum et al., 2014; Chu et al., 2015; Ngambenjawong & Pun, 2017) has shown that varying these structural parameters appreciably impacts peptide stability and interactions with biological molecules, aggregates, and membranes. Yet, therapeutic peptides differ widely from one another in length, structure, composition, and mechanism of action. Some peptides lose appreciable function upon conjugation to polymers, and in these cases are more suited to physical (e.g., non-covalent) encapsulation in polymers. All of these factors are likely to influence these structure-property-performance relationships. It is imperative that, to realize the vast potential of therapeutic peptides, the impacts of peptide and polymer composition on the conjugate structure, properties, and biomolecular interactions are understood.

A major thrust of the presently disclosed subject matter is to systematically control how peptide presentation within polymers determines biophysical properties-size, shape, and surface charge, loading and release of peptide (in the case of encapsulated peptides), and biomolecular interactions. Using highly accessible, tunable, and scalable polymer and peptide chemistry as a versatile platform, the presently disclosed subject matter relates to the synthesis and biophysical evaluation/use of peptide-polymer biophysical properties to build therapeutically relevant conjugates: design, synthesis, and characterization of polymers and polymer-peptide conjugates with complex architectures (Parelkar et al., 2014; Ghobadi et al., 2016; Zigmond et al., 2016; Johnston et al., 2017) and supramolecular assembly elements (Chang et al., 2015; Kalasin et al., 2017; Fan et al., 2017; Letteri et al., 2017; Song et al., 2017a; Santa Chalarca et al., 2018; Dong et al., 2019; Li et al., 2019; Dong et al., 2020; Lin et al., 2020; Grewal et al., 2021; Song et al., 2021).

### II. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience.

Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

A disease or disorder is “alleviated” if the severity of a symptom of the disease, condition, or disorder, or the frequency with which such a symptom is experienced by a subject, or both, are reduced by any measurable criterion. In some embodiments, a disease or disorder is “alleviated” if the severity of a symptom of the disease, condition, or disorder, or the frequency with which such a symptom is experienced by a subject, or both, are reduced to a condition that would be considered to be normal (i.e., absent).

As used herein, the term “subject” refers to an individual (e.g., human, animal, or other organism) to be treated by the methods or compositions of the present invention. Subjects include, but are not limited to, mammals (e.g., murines, simians, equines, bovines, porcines, canines, felines, and the like), and includes humans. In the context of the invention, the term “subject” generally refers to an individual who will receive or who has received treatment for a condition characterized by the presence of bacteria (e.g., Bacillus anthracis (e.g., in any stage of its growth cycle), or in anticipation of possible exposure to bacteria. As used herein, the terms “subject” and “patient” are used interchangeably, unless otherwise noted.

As used herein, the terms “neutralize” and “neutralization” when used in reference to bacterial cells or spores (e.g., B. anthracis cells and spores) refers to a reduction in the ability of the spores to germinate and/or cells to proliferate.

As used herein the term “bacterial spore” or “spore” is used to refer to any dormant, non-reproductive, but viable structure produced by some bacteria (e.g., Bacillus and Clostridium) in response to adverse environmental conditions.

As used herein, the term “treating a surface” refers to the act of exposing a surface to one or more compositions of the present invention. Methods of treating a surface include, but are not limited to, spraying, misting, submerging, wiping, and coating. Surfaces include organic surfaces (e.g., food products, surfaces of animals, skin, etc.) and inorganic surfaces (e.g., medical devices, countertops, instruments, articles of commerce, clothing, etc.).

As used herein, the terms “effective amount” and “therapeutically effective amount” are used interchangeably and refer to the amount that provides a therapeutic effect, e.g., an amount of a composition that is effective to treat or prevent pathological conditions, including signs and/or symptoms of disease, associated with a pathogenic organism infection (e.g., spore germination, bacterial growth, toxin production, etc.) in a subject.

The terms “bacteria” and “bacterium” refer to all prokaryotic organisms, including those within all of the phyla in the Kingdom Procaryotae. As used herein, the term “microorganism” refers to any species or type of microorganism, including but not limited to, bacteria, archaea, fungi, protozoans, mycoplasma, and parasitic organisms.

As used herein the term “colonization” refers to the presence of bacteria in a subject that are either not found in healthy subjects, or the presence of an abnormal quantity and/or location of bacteria in a subject relative to a healthy patient.

The term “stationary growth phase” as used herein defines the growth characteristics of a given population of microorganisms. During a stationary growth phase, the population of bacteria remains stable with the rate of bacterial division being approximately equal to the rate of bacterial death. This can be due to increased generation time of the bacteria. Accordingly, “stationary phase bacteria” are bacteria that are in a stationary growth phase. “Exponential phase bacteria” are bacteria that are rapidly proliferating, and the population is rapidly expanding, typically the number of bacteria increases at an exponential rate.

As used herein a “multidrug-resistant” (or “MDR”) microorganism or bacteria is an organism that has an enhanced ability, relative to non-resistant strains, to resist distinct drugs or chemicals (of a wide variety of structure and function) targeted at eradicating the organism. Typically, the term refers to resistance to at least 3 classes of antibiotics.

Chemokines are small proteins secreted by cells that have the ability to induce directed chemotaxis in responsive cells. As used herein the term “interferon-inducible (ELR) CXC chemokine” refers to a chemokine protein, or corresponding peptidomimetic, having a motif of four conserved cysteine residues, the first two of which are separated by a non-conserved amino acid (thus constituting the Cys-X-Cys or ‘CXC’ motif) and devoid of a three amino acid sequence, glutamic acid-leucine-arginine (the ‘ELR’ motif), immediately proximal to the CXC sequence. Examples of interferon-inducible (ELR) CXC chemokines include human CXCL9, murine CXCL9, human CXCL10, murine CXCL10, human CXCL11, and murine CXCL11.

CXCL9, CXCL10, and CXCL11 are potently induced by both type 1 and type 2 interferons (IFN-α/β and IFN-γ, respectively).

As used herein, the term “adjuvant” as used herein refers to an agent which enhances the pharmaceutical effect of another agent.

The expression “amino acid” as used herein is meant to include both natural and synthetic amino acids, and both D- and L-amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage can be present or absent in the peptides of the invention.

The term “amino acid” is used interchangeably with “amino acid residue” and can refer to a free amino acid and to an amino acid residue of a peptide. It will be apparent from the context in which the term is used whether it refers to a free amino acid or a residue of a peptide.

The term “antibody”, as used herein, refers to an immunoglobulin molecule which is able to specifically bind to a specific epitope on an antigen. Antibodies can be derived from natural sources or from recombinant sources and can be intact immunoglobulins or immunoreactive portions of intact immunoglobulins (for example, a fragment or derivative of an antibody that includes an antigen-binding site or a paratope). Antibodies are typically tetramers of immunoglobulin molecules. The antibodies in the present invention can exist in a variety of forms including, for example, polyclonal antibodies, monoclonal antibodies, Fv, Fab and F (ab) 2, as well as single chain antibodies and humanized antibodies (see e.g., Bird et al., 1988; Harlow & Lane, 1988; Houston et al., 1988; Harlow & Lane, 1999; each of which is incorporated herein by reference in its entirety).

The term “synthetic antibody” as used herein refers to an antibody which is generated using recombinant DNA technology, such as, for example, an antibody expressed by a bacteriophage or a host cell. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antimicrobial agent”, as used herein, refers to any entity that exhibits antimicrobial activity, i.e. the ability to inhibit the growth of and/or kill bacteria, including for example the ability to inhibit growth or reduce viability of bacteria by at least about 30%, at least about 40%, at least about 50%, at least about 60%, at least about 70% or more than 70%, as compared to bacteria not exposed to the antimicrobial agent. The antimicrobial agent can exert its effect either directly or indirectly and can be selected from a library of diverse compounds, including for example antibiotics. For example, various antimicrobial agents act, inter alia, by interfering with (1) cell wall synthesis, (2) plasma membrane integrity, (3) nucleic acid synthesis, (4) ribosomal function, and (5) folate synthesis. One of ordinary skill in the art will appreciate that a number of “antimicrobial susceptibility” tests can be used to determine the efficacy of a candidate antimicrobial agent.

As used herein, the term “antisense oligonucleotide” means a nucleic acid polymer, at least a portion of which is complementary to a nucleic acid which is present in a normal cell or in an affected cell. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (see e.g., U.S. Pat. No. 5,034,506 to Summerton & Weller; Nielsen et al., 1991). The term “antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences.

As used herein, the term “biologically active fragments” or “bioactive fragment” of a polypeptide encompasses natural or synthetic portions of the full-length protein that are capable of specific binding to their natural ligand or of performing the function of the protein.

A “pathogenic” cell is a cell which, when present in a tissue, causes or contributes to a disease or disorder in the animal in which the tissue is located (or from which the tissue was obtained).

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T”, is complementary to the sequence “T-C-A.”

The term “complex”, as used herein in reference to proteins, refers to binding or interaction of two or more proteins. Complex formation or interaction can include such things as binding, changes in tertiary structure, and modification of one protein by another, such as phosphorylation.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

As used herein, the term “cytokine” refers to an intercellular signaling molecule, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

As used herein, a “derivative” of a compound refers to a chemical compound that can be produced from another compound of similar structure in one or more steps, as in replacement of H by an alkyl, acyl, or amino group. Similarly, a “derivative” of a peptide (or of a polypeptide) is a compound that can be produced from or has a biological activity similar to a peptide (or a polypeptide) but that differs in the primary amino acid sequence of the peptide (or the polypeptide) to some degree. By way of example and not limitation, a derivative of a subject peptide of the presently disclosed subject matter is a peptide that has a similar although not identical primary amino acid sequence as the subject peptide (for example, has one or more amino acid substitutions) and/or that has one or more other modifications (e.g., N-terminal, C-terminal, and/or internal modifications) as compared to the subject peptide. Thus, the term “derivative” compasses the term “modified peptide” and vice versa, in the context of peptides. In some embodiments, a derivative of a peptide is a C-terminal amidated peptide.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid of the amino acid sequence, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

The terms “formula” and “structure” are used interchangeably herein.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool; Altschul et al., 1993) are available for determining sequence identity.

In some embodiments, “identity” can be expressed as a “percent identity”. As used herein, the phrase “percent identity” in the context of two nucleic acid or polypeptide sequences, refers to two or more sequences or subsequences that have in some embodiments 60%, in some embodiments 70%, in some embodiments 75%, in some embodiments 80%, in some embodiments 85%, in some embodiments 90%, in some embodiments 92%, in some embodiments 94%, in some embodiments 95%, in some embodiments 96%, in some embodiments 97%, in some embodiments 98%, in some embodiments 99%, and in some embodiments 100% nucleotide or amino acid residue identity, respectively, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments, the percent identity exists over at least about 150 residues. In some embodiments, the percent identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm disclosed in Smith & Waterman, 1981; by the homology alignment algorithm disclosed in Needleman & Wunsch, 1970; by the search for similarity method disclosed in Pearson & Lipman, 1988; by computerized implementations of these algorithms (GAP, BESTFIT, FASTA, and TFASTA in the GCG® WISCONSIN PACKAGE®, available from Accelrys, Inc., San Diego, California, United States of America), or by visual inspection.

See generally, Altschul et al., 1990; Ausubel et al., 2002; and Ausubel et al., 2003.

One example of an algorithm that is suitable for determining percent sequence identity and sequence similarity is the BLAST algorithm, which is described in Altschul et al., 1990. Software for performing BLAST analysis is publicly available through the website of the National Center for Biotechnology Information. This algorithm involves first identifying high scoring sequence pairs (HSPs) by identifying short words of length W in the query sequence, which either match or satisfy some positive valued threshold score T when aligned with a word of the same length in a database sequence. T is referred to as the neighborhood word score threshold. See generally, Altschul et al., 1990. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences (see e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

The term “inhibit”, as used herein, refers to the ability of a compound or any agent to reduce or impede a described function or pathway. For example, inhibition can be by at least 10%, by at least 25%, by at least 50%, by at least 75%, by at least 80%, by at least 85%, by at least 90%, by at least 95%, by at least 97%, by at least 99%, or more.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound invention or be shipped together with a container which contains the identified compound. Alternatively, the instructional material can be shipped separately from the container with the intention that the instructional material and the compound be used cooperatively by the recipient.

An “isolated” compound/moiety is a compound/moiety that has been removed from components naturally associated with the compound/moiety. For example, an “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “modulate”, as used herein, refers to changing the level of an activity, function, or process. The term “modulate” encompasses both inhibiting and stimulating an activity, function, or process.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in an animal. In some embodiments, a pharmaceutically acceptable carrier is pharmaceutically acceptable for use in a human.

The term “polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides (e.g., a polypeptide of in some embodiments at least 50 amino acids, in some embodiments at least 75 amino acids, in some embodiments at least 100 amino acids, in some embodiments at least 200 amino acids, in some embodiments at least 300 amino acids, in some embodiments at least 500 amino acids, and in some embodiments more than 500 amino acids).

A peptide encompasses a sequence of 2 or more amino acids wherein the amino acids are naturally occurring or synthetic (non-naturally occurring) amino acids.

The term “linked” or like terms refers to a connection between two entities. The linkage can comprise a covalent, ionic, or hydrogen bond or other interaction that binds two compounds or substances to one another.

As used herein the term “peptidomimetic” refers to a chemical compound having a structure that is different from the general structure of an existing peptide, but that functions in a manner similar to the existing peptide, e.g., by mimicking the biological activity of that peptide. The term “modified peptide” encompasses a peptidomimetic. Peptidomimetics typically comprise naturally occurring amino acids and/or unnatural amino acids, but can also comprise modifications to the peptide backbone. For example, a peptidomimetic can include one or more of the following modifications:
1. Peptides wherein one or more of the peptidyl-C(O) NR-linkages (bonds) have been replaced by a non-peptidyl linkage such as a —CH₂-carbamate linkage (—CH₂OC(O)NR—), a phosphonate linkage, a —CH₂-sulfonamide linkage (—CH₂—S(O)₂NR—) linkage, a urea (—NHC(O)NH—) linkage, a —CH₂-secondary amine linkage, an azapeptide bond (CO substituted by NH), or an ester bond (e.g., depsipeptides, wherein one or more of the amide (—CONHR—) bonds are replaced by ester (COOR) bonds) or with an alkylated peptidyl linkage (—C(O)NR—) wherein R is C₁-C₆ alkyl;
2. Peptides wherein the N-terminus is derivatized to a —NRR1 group, to a —NRC(O)R group, to a —NRC(O)OR group, to a —NRS(O)₂R group, to a —NHC(O)NHR group where R and R1 are hydrogen or C₁-C₆ alkyl with the proviso that R and R1 are not both hydrogen;
3. Peptides wherein the C terminus is derivatized to —C(O)R2 where R2 is selected from the group consisting of C₁-C₆ alkoxy, and —NR3R4 where R3 and R4 are independently selected from the group consisting of hydrogen and C₁-C₄ alkyl;
4. Modification of a sequence of naturally occurring amino acids with the insertion or substitution of a non-peptide moiety, e.g., a retroinverso fragment.

The term “permeability”, as used herein, refers to transit of fluid, cell, or debris between or through cells and tissues.

A “sample”, as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds”, as used herein, is meant a compound which recognizes and binds a specific protein, but does not substantially recognize or bind other molecules in a sample, or it means binding between two or more proteins as in part of a cellular regulatory process, where said proteins do not substantially recognize or bind other proteins in a sample.

The term “standard”, as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse, and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

As used herein an “amino acid modification” refers in some embodiments to a substitution, addition, or deletion of an amino acid, and includes substitution with, or addition of, any of the 20 amino acids commonly found in human proteins, as well as unusual or non-naturally occurring amino acids such as but not limited to D-amino acids. Commercial sources of unusual amino acids include Sigma-Aldrich (Milwaukee, Wisconsin, United States of America), ChemPep Inc. (Miami, Florida, United States of America), and Genzyme Pharmaceuticals (Cambridge, Massachusetts, United States of America). Unusual amino acids can be purchased from commercial suppliers, synthesized de novo, or chemically modified or derivatized from naturally occurring amino acids. Amino acid modifications include linkage of an amino acid to a conjugate moiety, such as a hydrophilic polymer, acylation, alkylation, and/or other chemical derivatization of an amino acid. The term “modified peptide” encompasses any amino acid modification as described herein.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substitutions can be designed based on, for example, the model of Dayhoff et al., 1978.

In some embodiments, an amino acid substitution is a conservative amino acid substitution. As used herein, the term “conservative amino acid substitution” is defined in some embodiments as exchanges within one of the following five groups:
I. Small aliphatic, nonpolar, or slightly polar residues: Ala, Ser, Thr, Pro, Gly;
II. Polar, charged residues and their amides: Asp, Asn, Glu, Gln, His, Arg, Lys;
III. Large, aliphatic, nonpolar residues: Met Leu, Ile, Val, Cys
IV. Large, aromatic residues: Phe, Tyr, Trp

Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., 1990.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, amino acids whose hydropathic indices are in some embodiments within +/−2, in some embodiments within +/−1, and in some embodiments within +/−0.5 can be employed.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, in some embodiments an amino acid with a compact side chain, such as glycine or serine, would not be replaced with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet, or reverse turn secondary structure has been determined and is known in the art (see e.g., Chou & Fasman; 1974; Chou & Fasman, 1978; Chou & Fasman, 1979).

Based on such considerations and extensive empirical study, tables of conservative amino acid substitutions have been constructed and are known in the art. By way of example and not limitation, the following substitutions can be made: arginine and lysine; glutamate and aspartate; serine and threonine; glutamine and asparagine; and valine, leucine, and isoleucine. Alternatively, Table 1 lists exemplary conservative amino acid substitutions.

TABLE 1
Exemplary Conservative Amino Acid Substitutions

| Amino Acid | Possible Substitution(s) |
|---|---|
| Ala (A) | Leu, Ile, Val |
| Arg (R) | Gln, Asn, Lys |
| Asn (N) | His, Asp, Lys, Arg, Gln |
| Asp (D) | Asn, Glu |
| Cys (C) | Ala, Ser |
| Gln (Q) | Glu, Asn |
| Glu (E) | Gln, Asp |
| Gly (G) | Ala |
| His (H) | Asn, Gln, Lys, Arg |
| Ile (I) | Val, Met, Ala, Phe, Leu |
| Leu (L) | Val, Met, Ala, Phe, Ile |
| Lys (K) | Gln, Asn, Arg |
| Met (M) | Phe, Ile, Leu |
| Phe (F) | Leu, Val, Ile, Ala, Tyr |
| Pro (P) | Ala |
| Ser (S) | Thr |
| Thr (T) | Ser |
| Trp (W) | Phe, Tyr |
| Tyr (Y) | Trp, Phe, Thr, Ser |
| Val (V) | Ile, Leu, Met, Phe, Ala |

In some embodiments, another consideration for amino acid substitutions include whether or not the residue is located in the interior of a protein or is solvent exposed. For interior residues, conservative substitutions can include in some embodiments: Asp and Asn; Ser and Thr; Ser and Ala; Thr and Ala; Ala and Gly; Ile and Val; Val and Leu; Leu and Ile; Leu and Met; Phe and Tyr; Tyr and Trp. For solvent exposed residues, conservative substitutions can include in some embodiments: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, the Dayhoff matrix, the Grantham matrix, the McLachlan matrix, the Doolittle matrix, the Henikoff matrix, the Miyata matrix, the Fitch matrix, the Jones matrix, the Rao matrix, the Levin matrix, and the Risler matrix (summarized in, for example, Johnson & Overington, 1993; see also the PROWL resource available at the website of The Rockefeller University, New York, New York, United States of America).

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### III. Exemplary Embodiments

How can antimicrobial peptides and neutral hydrophilic groups be arranged on polymers to optimize antimicrobial performance? Antimicrobial peptides often combine cationic and hydrophobic character to interact with and disrupt the anionic lipid membranes of bacteria. Unfortunately, these features also cause toxicity to mammalian cells. Conjugation of AMPs to polymers significantly reduces toxicity-promoting interactions with the mammalian cells and can improve AMP solubility, shield peptides from degradation, and yield larger molecules that avoid rapid renal filtration to prolong circulation in the bloodstream (Imura et al., 2007). Yet, these improvements often come at the expense of bactericidal activity, for example with the polymer surrounding the peptide and preventing interaction with bacterial membranes. The literature on AMP-polymer conjugates overwhelmingly and consistently underscores the role of molecular architecture (e.g., star, comb, and hyperbranched conjugates, and variations thereof) on conjugate antimicrobial performance (Cui et al., 2021) yet the field lacks a systematic understanding of the impacts of AMP-polymer conjugate architecture on molecular properties and antimicrobial performance. As disclosed herein, molecular architecture, peptide density and orientation, and chain length of polymers combining AMPs and neutral hydrophilic groups are varied to determine the effects of these structural variables on biophysical properties relevant for antimicrobial performance.

It is suspected that comb and hyperbranched architectures capable of presenting both AMPs and neutral hydrophilic groups optimize performance by providing a high local concentration of AMPs to bolster antimicrobial activity together with toxicity-mitigating hydrophilic groups. Specifically, increasing AMP density and polymer chain length may increase antimicrobial activity; for example, enhanced biophysical and antimicrobial properties of conjugates combining naturally derived AMPs with neutral hydrophilic groups arranged in linear, star, comb, and hyperbranched architectures. Chain length, as well as peptide density and orientation are also varied to improve biophysical properties and antimicrobial performance.

As a general approach, to prepare AMPs for incorporation into polymers, the N-terminal amine is functionalized with a polymerizable methacrylamide group. After chromatographic purification to >95%, mass spectrometry and nuclear magnetic resonance (NMR) spectroscopy are employed to confirm the primary peptide structure. Adapting reported methods for the co-polymerization of methacrylamide-functionalized peptides with the neutral hydrophilic monomer 2-hydroxypropyl methacrylamide (HPMA; Yang et al., 2006; Johnson et al., 2010; Yang & Kopecek, 2016; Song et al., 2017b), comb polymers featuring 5-20 mol % peptide pendent groups are generated. NMR spectroscopy and size exclusion chromatography (SEC) allow for the confirmation of the composition and molecular weight of the conjugates. The AMP secondary structure (e.g., α-helix vs. random coil) pre- and post-conjugation are determined in a membrane-mimetic environment using circular dichroism spectroscopy. With dynamic light scattering, gel electrophoresis, zeta potential measurements, and transmission electron microscopy, the supramolecular assembly of the conjugates in aqueous solution are determined by measuring the size, surface charge, and morphology. Constructing a multiscale picture of AMP-polymer conjugate structure is important for connection to peptide stability, which is assessed by high-performance liquid chromatography (HPLC) after incubation with proteases, and antimicrobial performance. The antimicrobial performance of the conjugates are determined, screening initially for antimicrobial activity against a panel of multidrug-resistant pathogenic bacterial strains using an ALAMARBLUE™-based viability assay, followed by determination of the minimum inhibitory concentrations for active conjugates according to standard methodology. Hemolysis assays are used to gauge toxicity to mammalian cells.

Successful completion of this work furnishes candidate antibiotic formulations as well as design criteria, structure-property relationships, and methods for presenting antimicrobial peptides on polymer scaffolds to overcome barriers to clinical translation. In addition to the structural variables discussed above, it can also be important to vary the identity of the linker connecting the AMP to the polymer backbone (e.g., to hydrocarbon rather than glycine-based spacers capable of hydrogen bonding) and the neutral hydrophilic monomer (e.g., to polyethylene glycol or zwitterions). Generating hyperbranched conjugates to enable a comparison among comb, hyperbranched, star, and linear conjugate architectures is also envisioned.

How does the density and arrangement of neutral hydrophilic groups within charged polymers modulate encapsulation, stability, and release of charged therapeutic peptides? While some therapeutic peptides retain activity upon conjugation to polymers, others are less tolerant to chemical modification. In this case, physical encapsulation provides an excellent approach to providing some of the same benefits as conjugation, namely stabilization against clearance/degradation and increased local concentrations of a given peptide, without chemical modification (Borro & Malmsten, 2019). Electrostatic complexation is well established for complexation of nucleic acids in gene therapy (Johnson et al., 2010; Johnson et al., 2011) and for the generation of polyelectrolyte complexes from mixtures of model charged peptides (Perry et al., 2015; Priftis et al., 2015; Chang et al., 2017; Lytle et al., 2019; Sing & Perry, 2020). There remain, however, far fewer examples of electrostatic complexes for encapsulation and release of therapeutic peptides (Kizilbey et al., 2018; Borro & Malmsten, 2019; Wang et al., 2019; Raveendran et al., 2020) which can vary appreciably in composition. As an example of the benefits of electrostatic encapsulation of peptides, loading the cationic anticancer peptide melittin within polyelectrolyte complexes functionalized with cancer cell-targeting moieties imparted the desired cytotoxic effects to cancer cells and provided protection against enzymatic degradation (Raveendran et al., 2020). By optimizing the density, arrangement, and type of neutral hydrophilic groups within charged polymers, the stability and the loading capacity and release profiles of antimicrobial and anti-inflammatory cationic peptide therapeutics can be controlled.

As disclosed herein, design rules are built that inform encapsulation of therapeutic peptides. Together, the proposed systematic studies involving the arrangement of therapeutic peptides with neutral hydrophilic groups on polymers elucidate critically needed guidelines for designing polymers to present therapeutic peptides to optimize performance. The presently disclosed subject matter thus connects the structural and compositional aspects of these polymer-peptide formulations in order to finely control with advanced, yet highly accessible synthetic methods to biophysical properties (i.e., size, morphology, surface charge of the polymer-peptide formulations, binding interactions, and loading and release profiles).

Stereochemistry-driven complexation, or “stereocomplexation” of macromolecules produces remarkable enhancements in the stability and thermomechanical properties of materials (Slager & Domb, 2003; Tsuji, 2016; Worch et al., 2019). For instance, whereas poly(D-lactide) and poly(L-lactide) melt at 180° C., the melting temperature of their stereocomplexed blends (1:1 ratio of poly(D-lactide): poly(L-lactide)) is nearly 50° C. higher (Tsuji, 2016). One of the main goals of the presently disclosed subject matter is to expand macromolecular stereocomplexation to new materials and application spaces using D-peptides and L-peptides. By elucidating the effects of peptide structure on interactions between complementary stereoregular peptides (i.e., D- and L-peptides), developing peptide stereocomplexes as supramolecular cross-linkers for hydrogels and, relevant to the presently disclosed subject matter, as ligands to specifically bind proteins is facilitated.

Reasoning that since natural proteins are comprised of L-amino acids, there are opportunities to design D-peptide segments to specifically stereocomplex given protein segments. Compared to completely L-type peptides and proteins (e.g., antibodies), it is anticipated that D-peptides and peptide stereocomplexes will facilitate specific binding with improved proteolytic stability, an outstanding challenge in biologic-based therapeutics (Collier & Segura, 2011; Foster et al., 2017; Zamuner et al., 2017; Bila et al., 2019). While recognizing that the immune response to D-peptides is complex (Sela & Zisman, 1997; Li et al., 2016; Arranz-Gibert et al., 2018), reports attesting to their favorable immune system activation and improved stability (Sela & Zisman, 1997; Li et al., 2016; Arranz-Gibert et al., 2018; Ran et al., 2020; Griffin et al., 2021) are encouraging. Thus, stereochemistry-driven interactions can be considered in designing protein binding groups for targeted drug delivery as well as inhibition or sequestration of problematic peptides or proteins (e.g., amyloid β plaques implicated in Alzheimer's disease (Chiti & Dobson, 2006) and toxic dipeptide repeats characteristic of Amyotrophic Lateral Sclerosis (Mori et al., 2013; Shi et al., 2017).

Fully appreciating the potential of therapeutic peptides and the role of polymers in enabling their clinical implementation, the tunability of polymer and peptide chemistry is leveraged to maximize the benefit of polymers to therapeutic peptides. solid-phase peptide synthesis (Collier & Segura, 2011), and controlled polymerization methods are employed to achieve precision control of chain length and composition so as to connect structure to therapeutically relevant biophysical properties. As disclosed herein, controlled radical polymerization are used to generate comb-shaped polymer-peptide conjugates that display antimicrobial and poly(dipeptide)-binding peptides and neutral hydrophilic groups pendent to a synthetic polymer chain. These polymerization methods afforded independent control of chain length and peptide density, allowing for the probing of the effects of these structural variables on conjugate size, morphology, surface charge, stability, and interactions with biological targets. Also, by modulating the amount and arrangement of neutral hydrophilic groups within anionic polymers using controlled radical polymerization, the loading capacity and release rate of cationic antimicrobial and anti-inflammatory peptides is tuned.

Since not all peptides are amenable to chemical conjugation without loss of function, anionic polymers are tailored to electrostatically encapsulate cationic antimicrobial and anti-inflammatory peptides and facilitate programmable release. Dispersing neutral hydrophilic groups randomly into anionic polymers may decrease binding affinity and proteolytic stability and increase release rate, whereas spatially separating neutral hydrophilic and anionic segments in block copolymers increases stability and slow release of encapsulated peptides.

As an exemplary general approach, random and block copolymers consisting of anionic and neutral hydrophilic groups for electrostatic encapsulation of cationic peptides are prepared using controlled radical polymerization. Block copolymers are prepared by chain extension of a polyanion with a neutral hydrophilic monomer. Before and after complexation with cationic peptides, multiscale structural characterization of the polymers is conducted. Using isothermal titration calorimetry (ITC), gel electrophoresis, and turbidimetry, electrostatic complexation of the various polyanions to the antimicrobial peptide is measured. Proteolytic stability, loading capacity, and release profiles of peptides with the anionic polymers are characterized using HPLC and spectrometry. The binding affinity, loading and release profiles, proteolytic stability, and complex morphology and composition are connected to biological performance. The antimicrobial performance of peptide-polymer formulations is then evaluated using bacteriologic assays.

While the copolymers combining anionic and neutral hydrophilic groups are a good starting point, to increase peptide stability and prolong release, hydrophobic monomers can be incorporated. To accelerate release, degradable poly(aminoester) s can be employed. The degradation of these polymers was characterized as a function of hydrophobic content, buffer concentration, and pH (Kuenen et al., 2021), and anionic variants of these cationic polymers suitable for encapsulation of cationic peptides using click chemistry to install two anionic thiols per repeating unit were recently prepared. An exemplary degradable poly(aminoester) is a net anionic poly(β-amino ester) s (PBAEs) as described in Kuenen et al., 2023. As set forth therein, PBAEs can be employed for complexing cationic cargo, including but not limited to the stapled peptides, truncated versions thereof, conjugates thereof, including but not limited to the linear conjugates, comb-shaped conjugates, bottlebrush-shaped conjugates, star-shaped conjugates, and combinations thereof disclosed herein.

### Pharmaceutical Compositions

In some embodiments, the peptides and/or peptide-polymer conjugates of the presently disclosed subject matter are present in a pharmaceutical composition. Thus, in some embodiments the presently disclosed subject matter relates to pharmaceutical compositions comprising, consisting essentially of, or consisting of one or more peptides as disclosed herein, one or more conjugates as disclosed herein, one or more polymers as disclosed herein, or any combination thereof, along with one or more pharmaceutically acceptable carriers, diluents, or excipients. In some embodiments, the presently disclosed pharmaceutical compositions are pharmaceutically acceptable for use in humans.

Thus, the disclosed pharmaceutical compositions can be employed by administration to a subject in need thereof. In some embodiments, the disclosed pharmaceutical compositions can be administered in vivo in a pharmaceutically acceptable carrier. By “pharmaceutically acceptable” is meant a material that is not biologically or otherwise undesirable, i.e., the material can be administered to a subject, along with a peptide composition of the presently disclosed subject matter, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution and/or in suspension (for example, incorporated into microparticles, liposomes, and/or cells.

As would be understood by those of skill in the art, when peptides are synthesized, they are typically prepared with counter-ions to stabilize basic/acidic sidechains of amino acid residues. Thus, peptide drugs often occur in the form of salts where different counter-ions can affect peptide structure, function, etc. differently. In particular, cationic peptides like those disclosed herein are typically obtained as trifluoroacetate (TFA) salts. However, TFA can be toxic and have off-target effects making it an unfavorable drug formulation for some uses. To address these issues, in some embodiments TFA has been exchanged with acetate or formate, each of which is non-toxic and can be used in peptide pharmaceuticals.

The peptide, conjugate, and polymer compositions of the presently disclosed subject matter can be used therapeutically in combination with one or more pharmaceutically acceptable carriers.

Suitable carriers and their formulations are described in Remington et al. (1975) Remington's Pharmaceutical Sciences, 15th ed., Mack Pub. Co., Easton, Pennsylvania, United States of America. Typically, an appropriate amount of a pharmaceutically acceptable salt is used in the formulation to render the formulation isotonic. Examples of the pharmaceutically acceptable carrier include, but are not limited to, saline, Ringer's solution, and dextrose solution. The pH of the solution is in some embodiments from about 5 to about 8, and in some embodiments from about 7 to about 7.5. Further carriers include sustained release preparations such as semipermeable matrices of solid hydrophobic polymers containing the peptide compositions, which matrices are in the form of shaped articles, e.g., films, liposomes, or microparticles. It will be apparent to those persons skilled in the art that certain carriers can be selected depending upon, for instance, the route of administration and/or concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents, and the like, in addition to the molecule of choice. Pharmaceutical compositions can also include one or more active ingredients such as antimicrobial agents, anti-inflammatory agents, anesthetics, and the like.

The pharmaceutical composition can be administered in a number of ways depending on whether local or systemic treatment is desired, and on the area to be treated. Administration can occur topically (including ophthalmically, vaginally, rectally, intranasally), orally, by inhalation, or parenterally, for example by intravenous drip, subcutaneous, intraperitoneal, or intramuscular injection. The disclosed peptide compositions can be administered in some embodiments topically, intravenously, intraperitoneally, intramuscularly, subcutaneously, intracavity, or transdermally.

Preparations for parenteral administration can include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose, and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, antioxidants, chelating agents, and inert gases and the like.

Formulations for topical administration can include ointments, lotions, creams, gels, drops, suppositories, sprays, liquids, and powders. Conventional pharmaceutical carriers, aqueous, powder or oily bases, thickeners, and the like can also be employed, as desired.

Compositions for oral administration include powders or granules, suspensions or solutions in water or non-aqueous media, capsules, sachets, or tablets. Thickeners, flavorings, diluents, emulsifiers, dispersing aids, or binders can in some embodiments also be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, or phosphoric acid and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl, and aryl amines and substituted ethanolamines.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the Examples

Materials. Fmoc-1-Cys(Trt)-OH (≥98%), Fmoc-1-Gly-OH (≥98%), Fmoc-1-Pro-OH (≥ 98%), Fmoc-1-Glu(OtBu)-OH (≥98%), Fmoc-1-Ser(Trt)-OH (≥98%), Fmoc-1-Lys (Boc)-OH (≥98%), Fmoc-1-Ala-OH·H₂O (≥98%), Fmoc-1-Ile-OH (≥98%), Fmoc-1-Leu-OH (≥98%), Fmoc-1-Val-OH (≥98%), Fmoc-1-Arg(Pbf)-OH (≥98%), Fmoc-(s)-2-(4-pentenyl) Alanine-OH (≥97%), rink amide resin LS (0.5 mmol/g, 100-200 mesh), and oxyma pure (≥99%) were purchased from Advanced ChemTech. N,N-dimethylformamide (DMF, ≥99.8%), acetic anhydride (≥98%), piperidine (≥99%), diisopropyl carbodiimide (DIC, ≥99%), Grubbs Catalyst® Generation I (M102, ≥97%), 1,2-dichloroethane (DCE) (≥99%), dichloromethane (DCM, ≥99.5%), diethyl ether (≥99%), 2,2′-(ethylenedioxy) diethanethiol (DODT,≥95%), triisopropylsilane (TIPS, ≥98%), trifluoroacetic acid (TFA, ≥99%), acetonitrile (ACN, for HPLC, gradient grade, ≥99.9%), methoxy-PEG-maleimide (linear PEG maleimide, average Mₙ=2 KDa), 4-arm PEG-Maleimide (average Mₙ=10 KDa), 8-arm PEG-Maleimide (average Mₙ=20 KDa), 8-arm PEG-Maleimide (average Mₙ=10 KDa), deuterium oxide (D₂O), phosphate buffered saline (PBS), 2,2,2-trifluoroethanol (TFE, ≥99%), sodium trifluoroacetate (NaTFAc, ≥98%), dl-dithiothreitol (DTT, ≥98%), potassium phosphate monobasic (≥99%), potassium phosphate dibasic (≥98%), sodium dodecyl sulfate (SDS, ≥99%), Proteinase K from Tritirachium album, lysogeny broth (LB)-Lennox, sodium chloride (≥99%), RPMI 1640 medium, RPMI 1640 medium without phenol red, low-endotoxin bovine serum albuim (BSA), Triton-X 100, and Melittin were purchased from Sigma Aldrich. Alamar blue was purchased from ThermoFisher.

Instrumentation. Reverse-phase analytical high performance liquid chromatography

(HPLC) was performed on an Alliance system from Waters equipped with a XBRIDGER C18 column (4.6 mm×50 mm, 3 μm) at 35° C. with flow rate of 1 mL/min and a photodiode array detector (Waters 2489 UV/Visible) to assess the purity of the peptides and conjugates as well as to monitor the progress of conjugation reactions and dialysis. Preparative HPLC, equipped with a C18 column (30 mm×150 mm, 5 μm) at room temperature with flow rate of 25.52 mL/min and a photodiode array detector (Waters 2489 UV/Visible), was used to purify the crude peptides and conjugates. Samples were prepared in HPLC solvent (95% v/v water with 0.1% TFA and 5% v/v ACN with 0.1% TFA).

NMR spectroscopy was conducted on an 800 MHz Burke Avance III Varian NMR spectrometer in D₂O. Chemical shifts were referenced to the solvent residual peak at 4.79 ppm. Spectra were analyzed with MestReNova v14.3.2-32681.

Matrix-assisted laser desorption/ionization-time of flight (MALDI-TOF) mass spectrometry was performed on a Shimadzu MALDI-8030 system with a 200 Hz solid-state laser (355 nm). The instrument was calibrated with a standard MALDI calibration kit (TOFMix, containing Glu Fib, P14R, Cytochrome C, BSA, CHCA matrix, Sinipinic acid matrix, and DHB matrix, Shimadzu) (670 femtomoles/μL in 70% v/v ACN with 0.1% TFA). The sample solution (1 μL, 2 mg/mL in HPLC solvent) was coated with a α-cyano-4-hydroxycinnamic acid (CHCA) matrix (1 μL, 5 mg/mL in 70% v/v ACN with 0.1% TFA) on a plate and was thoroughly dried in air before measurement.

Size exclusion chromatography (SEC) was performed in TFE with 0.02 M NaTFAc at 0.3 mL/min using a Tosoh system equipped with two isocratic pumps (one for the sample, the second for the solvent reference), a degasser, an auto sampler, one 4.6 mm×35 mm TSKgel guard super AW-H column (bead diameter: 9 μm), two 6 mm×150 mm TSKgel super AWM-H linear analytical columns (beads diameter: 9 μm), and a refractive index detector. Number average molecular weight (Mₙ) and dispersity (Ð) were determined relative to poly(methyl methacrylate) (PMMA) standards. Samples were prepared with concentrations of 2-3 mg/ml with an injection volume of 20 μL.

Circular dichroism spectroscopy was run under N_{2(g)} in a 0.1 cm path length quartz-cell at 25° C. using a JASCO (Easton, Maryland, USA) J-1500 CD spectrophotometer with a Peltier thermostatted single-position cell holder. Samples of peptide or conjugates were prepared at 0.1 mg/mL in 10 mM phosphate buffer (PB, pH=7) or in 50% v/v TFE to mimic the hydrophobic environment on cell membrane or in 60 mM SDS to mimic the anionic membranes of bacteria. Spectra were obtained in triplicate on the same solution from 180-250 nm with a scanning speed of 50 nm/min and integration time of 1 s. Mean residue ellipticity=ellipticity (θ, mdeg)/[10×path length (1, cm)×peptide concentration (C, mol/L)×number of amino acid residues]. Conjugate concentrations were calculated based on average molecular weight determined from NMR spectra, including TFA counter ions on each cationic Lys and Arg residue as well as protons on each anionic Glu residue.

Dynamic light scattering (DLS) and zeta potential measurements were performed on a Malvern Zetasizer Ultra with 4.0 mW He Ne laser (633 nm) at 25° C. Samples were prepared at concentrations of 100, 200, and 400 μM peptide in 10 mM PBS (pH=7.4). Samples were filtered (0.45 μm) before measurement. Size measurements were performed in square DTS0012 cuvettes in triplicate. The hydrodynamic diameter of samples was reported as the Z-average from the software or calculated from diffusion coefficients from correlograms using a non-linear cumulant analysis. The diffusion barrier technique described by the manufacturer was used for zeta potential measurements to protect the peptide-based material and the electrodes in DTS1070 cuvettes. Buffer (0.8 mL) was pre-loaded in the cuvette and sample solution (50 μL) was slowly added to the bottom of the cuvette using a gel-loading pipette tip to minimize convective mixing. Zeta measurements were performed in triplicate and reported as average with standard deviation.

Transmission Electron Microscopy (TEM) was performed using a FEI Titan instrument operating at an accelerating voltage of 120 kV. Peptide and conjugates samples were prepared at peptide concentrations of 200 μM in 10 mM PBS (pH=7.4). Carbon-coated copper girds (300 mesh, Electron Microscopy Sciences) were pretreated with O_{2(g)} and Ar_{(g)} in a plasma cleaner for 30 s. Samples (3 μL) were added onto the grids for 1 min, blotted with filter paper by placing filter paper at the edge of the grid to remove excess solution, and washed by dabbing and blotting off a drop of DI water (10 μL) three times. The washed grids were dried for 1 min before staining with 2% aqueous uranyl acetate solution (3 L) for 1 min. Excess uranyl acetate solution was blotted off with filter paper and the samples were air dried before imaging. Images were taken at magnifications ranging from 43000-87000X, and the particle diameters were measured with ImageJ. Histograms were generated in Excel by counting 200 structures per sample.

Peptide synthesis. Peptides (Ac-CGGP9: Ac-CGGPESKAIKNLLKAVSKERSKRSP, pre-stapled Ac-CGGP9: Ac-CGGPESKAIKA(p)LLKA(p)VSKERSKRSP, polymerizable P9 with an Ahx linker (mAhxP9): mAhxPESKAIKNLLKAVSKERSKRSP, GGGL8: GGGRTVRCTCI, Acm-protected GGGL8(Acm)₂: GGGRTVRC(Acm)TC(Acm)I, and Acm-protected L8(Acm)2GGC: RTVRC(Acm)TC(Acm)IGGC) were synthesized using an automated, microwave-assisted solid phase peptide synthesizer via Fmoc methods on Rink amide resin (loading capacity of 0.5 mmol/g). Fmoc-protecting groups on resin and amino acids were removed using 20% (v/v) piperidine in dimethylformamide (DMF). For the coupling of amino acids (0.2 M in DMF), diisopropylcarbodiimide (1 M in DMF) and Oxyma Pure (1 M in DMF) were added at 90° C. for 4 minutes. Acetyl capping of the N-terminus was achieved by performing a coupling reaction with acetic anhydride (10% v/v in DMF) at 60° C. Methacrylamide group was added to N-terminus by performing a coupling reaction with methacrylic anhydride (10% v/v in DMF) at 60° C. All arginine residues were coupled twice prior to the next amino acid to improve the purity of peptide.

Alternatively, pre-stapled Ac-P9: Ac-PESKAIKA(pentenyl)LLKA(pentenyl) VSKERSKRSP-NH₂ and pre-stapled Ac-CGGP9 used for synthesis of conjugation to PEG maleimide: Ac-CGGPESKAIKA(pentenyl)LLKA(pentenyl)VSKERSKRSP-NH₂ were synthesized using a CEM Liberty Blue microwave-assisted solid phase peptide synthesizer via standard Fmoc methods on a Rink amide resin (Advanced ChemTech). Fmoc-protecting groups on resin and amino acids were removed using 20% (v/v) piperidine in DMF at 90° C. for 70 s. For the coupling of amino acids (0.2 M in DMF), diisopropylcarbodiimide (DIC, 1 M in DMF) and Oxyma Pure (1 M in DMF) were added at 90° C. for 4 min. Acetyl capping of the N-terminal amine was achieved by reaction with acetic anhydride (10% v/v in DMF) at 65° C. for 2 min 30 s. All arginine residues were coupled twice prior to Fmoc-deprotection in an effort to prevent deletions.

Stapling procedure. Stapling was conducted via a ring-closing metathesis reaction between the pentenyl alanine residues in a 10 mM solution of Grubbs' first-generation catalyst in dichloroethane (DCE; see FIG. 21). Peptide-loaded resin was washed with DCE to remove DMF before reacting with the prepared catalyst solution on the synthesizer. The catalyst solution was degassed by bubbling N₂ at room temperature for 20 minutes before use. This mixture was added to reaction vessel to react for 30 minutes at 40° C., washed with DCE, and then reacted a second time under the same conditions.

Peptide deprotection. An acidic cleavage cocktail of 92.5% v/v TFA, 2.5% v/v TIPS, 2.5% v/v DODT, and 2.5% v/v DI water was used to cleave the peptides from resins and remove the side chain protecting groups from the peptide. After stirring the resin with deprotection cocktail for 3 hours at room temperature, peptide mixture (20 mL) was precipitated into cold diethyl ether in four centrifuge tubes (40 mL in each), isolated by centrifugation (4700 rpm, 5 minutes), washed with ether (×2), and dried under vacuum. The peptides were store in freezer (−20° C.) before use.

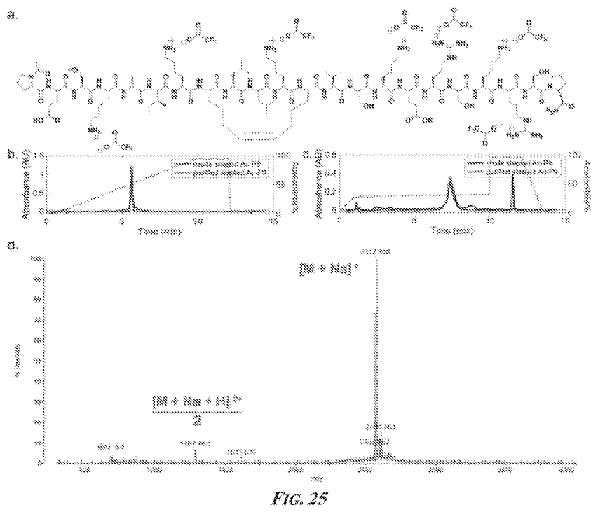

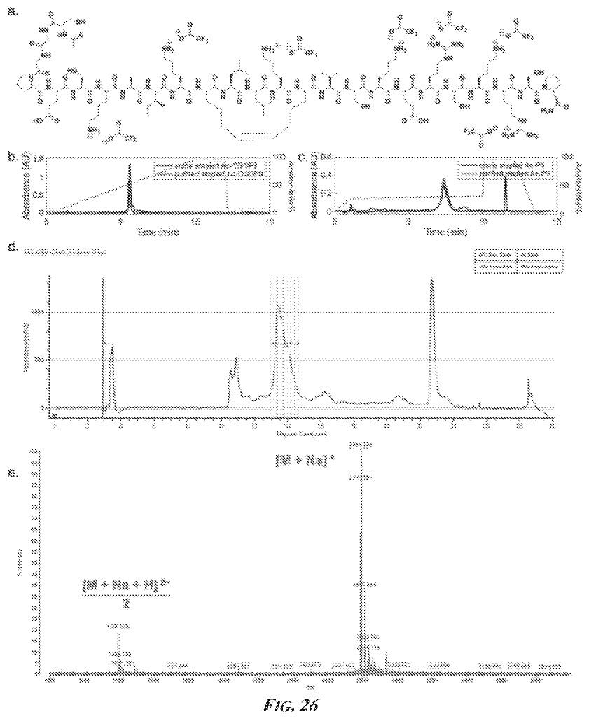

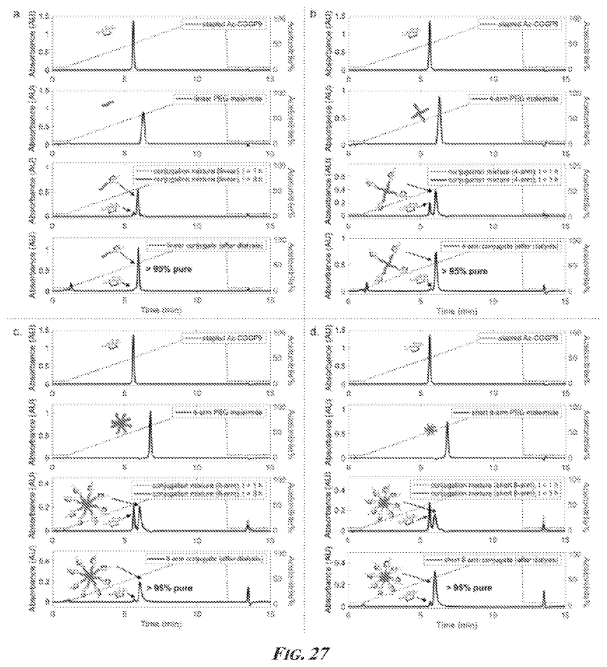

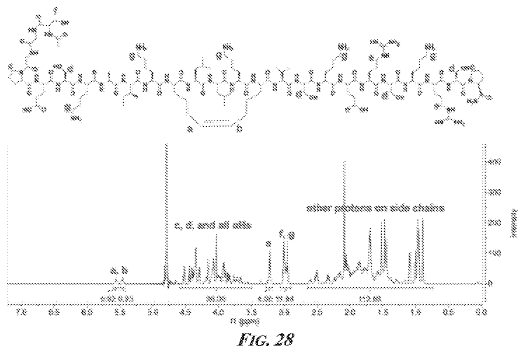

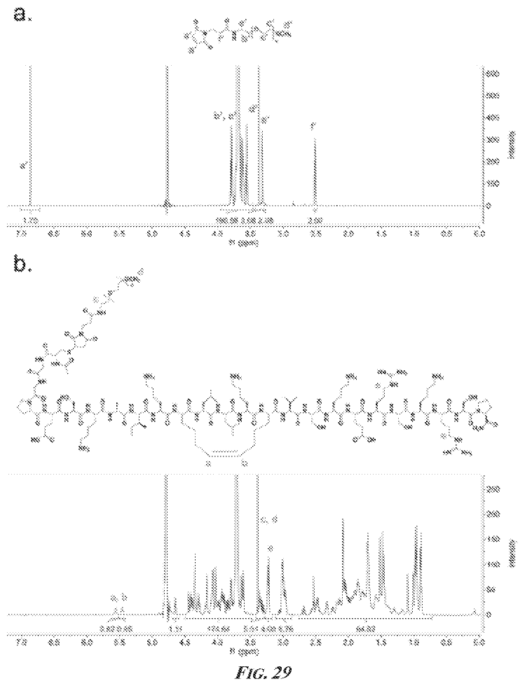

Peptide purification. Peptides were purified by preparative HPLC before conjugation and characterization. Crude peptides (30 mg) were dissolved in HPLC solvent (10 mL) and filtered through 0.45 μm PTFE syringe filters. The solution was injected, and the product was collected and lyophilized. The elution profiles used on the preparative HPLC were calculated by scaling up the column size, injection volume, flow rate, and loading mass from the corresponding analytical HPLC method as recommended by Waters. The recovery of the purification was calculated as the mass ratio of purified peptide to injected crude peptide, and therefore reflects both the purity of the crude peptide and the recovery from the chromatography system. The purity was determined by the % peak area on analytical HPLC, assuming similar extinction coefficients for the desired peptide and impurities. For each run, the recovery was ca. 30-40% from 30 mg crude peptide, with purity >99%. Synthesis and purification were confirmed by matrix-assisted laser desorption ionization (MALDI) time-of-flight (TOF) spectra (FIGS. 25 and 26). Stapled Ac-P9 m/z: [M+Na]⁺ calculated=2573.6; found=2573.0. Stapled Ac-CGGP9 m/z: [M+Na]⁺ calculated=2788.6; found=2789.3.

Conjugation of Stapled Ac-CGGP9 to Linear and Star-shaped PEG Maleimide. The purified peptide was conjugated to linear, 4-, 8-, and short 8-arm PEG maleimide. For the linear conjugate, purified stapled Ac-CGGP9 (with 7 TFA counter ions, 28.5 mg, 8 μmol) and mPEG maleimide (16 mg, 8 μmol, 1 equiv. to thiol group on peptide) were dissolved in 1×PBS (pH=7.4, 2 mL). For 4-arm star-shaped conjugate, 4-arm PEG maleimide (20 mg, 2 μmol) and stapled Ac-CGGP9 (with 7 TFA counter ions, 42.7 mg, 12 μmol, 1.5 equiv. to maleimide groups) were dissolved in 1×PBS (pH=7.4, 2 mL). For 8-arm star-shaped conjugate, 8-arm PEG maleimide (20 mg, 1 μmol) and stapled Ac-CGGP9 (with 7 TFA counter ions, 42.7 mg, 12 μmol, 1.5 equiv. to maleimide groups) were dissolved in 1×PBS (pH=7.4, 2 mL). For short 8-arm star-shaped conjugate, short 8-arm PEG maleimide (10 mg, 1 μmol) and stapled Ac-CGGP9 (with 7 TFA counter ions, 42.7 mg, 12 μmol, 1.5 equiv. to maleimide groups) were dissolved in 1×PBS (pH=7.4, 2 mL). The mixtures were stirred in 8 mL glass vials at room temperature for 4 h. The mixture (0.05 mL) was diluted in 0.95 mL HPLC eluent for HPLC analysis to monitor the reactions.

Considering that the cysteine on stapled Ac-CGGP9 may form disulfides with cysteines on other peptides and to yield dimers with a molecular weight of 5.5 kDa, the mixtures were with treated reducing agent DTT to break possible disulfide-linked unconjugated peptide and then dialyzed against water with 0.1% v/v TFA to remove unreacted peptide. For the star-shaped conjugates, membranes with MWCO of 3.5 kDa were used to keep the conjugates with molecular weight ranging from ~20-40 kDa, while for the linear conjugate mixture, a membrane with MWCO of 2 kDa was used to retain the ~4 kDa linear conjugate. Acidic conditions were selected to hinder disulfide formation and prevent supramolecular assembly during dialysis. To monitor the removal of unreacted peptide, the reaction mixture (0.05 mL) was diluted in 0.95 mL HPLC eluent for HPLC analysis. The peptides were not fully removed after one week. Hypothesizing that the polymer conjugates may complex the unreacted peptides such that they remain in the dialysis bag, 5% v/v ACN was added to solubilize and ‘release’ the unreacted peptides. We noted that adding 10% ACN accelerated the removal of unreacted peptide, while when adding more than 20% v/v ACN, we started to lose the conjugates with a low yield. The purified conjugates were lyophilized and stored at −20° C. until use. The yields of linear, 4-arm, 8-arm, and short 8-arm conjugates were 78%, 75%, 71%, and 70%, respectively, calculated with the assumption of full conversion of maleimide groups.

The mixtures were dried first and dissolved in water with 0.1% TFA (4 mL) before being dialyzed against water with 0.1% TFA (4 L) with membrane with MWCO of 2 kDa to remove the unreacted peptide in the mixture of conjugate. The acidic condition helped the linear conjugate which has similar size as the peptide to stay in the dialysis bag when removing the peptide. The mixture (0.05 mL) was diluted in 0.95 mL HPLC eluent for HPLC analysis to monitor the removal of unreacted peptide overnight. The purified conjugates were dried on a lyophilizer and stored at −20° C. before use.

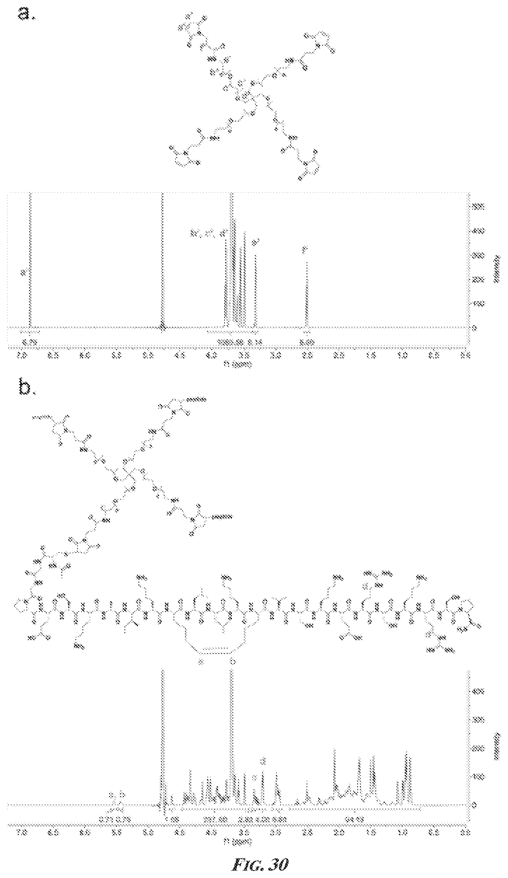

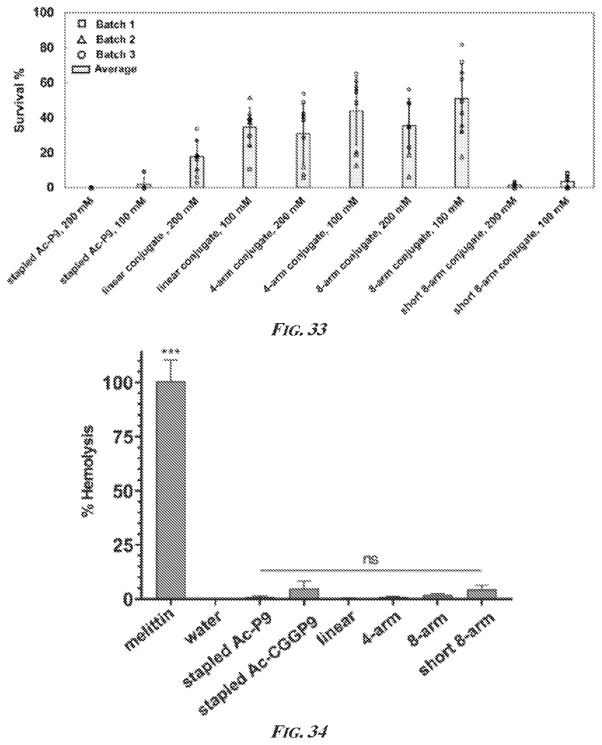

The peptide arm numbers on star-shaped conjugates were determined from ¹H NMR spectra (FIGS. 30-34). PEG maleimide with different architectures as well as the conjugates were dissolved in D₂O separately (5 mg/mL) and proton spectra were acquired with 128 scans. The integration of the Arg δH resonance at 3.22 ppm corresponding to 4H per peptide was set to 4 in the spectra of conjugates. The number of peptides per conjugate was calculated as the ratio of the integration of a polymer peak (δ=3.37 ppm, integration varied by polymer arm number) to the same peak on the conjugate.

Proteolytic Stability of Stapled Ac-CGGP9 and the Conjugates. Stock solutions of Proteinase K (5 μM, 0.14 mg/mL) and the peptides/conjugates (250 μM peptide) were prepared in either RPMI buffer or 1×PBS (pH=7.4) right before use. The mass concentration of stapled Ac-P9, stapled Ac-CGGP9, linear, 4-arm, 8-arm, and short 8-arm star-shaped conjugate stock solutions were 0.84, 0.89, 1.39, 1.78, 1.57, and 1.23 mg/mL, respectively. Stock solutions of peptide or conjugate (0.6 mL) were mixed with Proteinase K stock solution (0.6 mL) and filtered into a 2 mL HPLC sample vial before characterization. HPLC was used to monitor degradation by injecting 100 μL mixture after incubation in the presence or absence of Proteinase K for 0.5 h, 1 h, 2 h, and 24 h. Control samples were prepared by diluting the peptide or conjugate stock solution (0.6 mL) with RPMI buffer or PBS (0.6 mL). The fractions of peak area were calculated from HPLC by comparing the control samples to those treated with Proteinase K. To test if Proteinase K was still functional after the 24 h experiment, we spiked more peptide into the mixture in RPMI, in which stapled Ac-P9 powder (0.2 mg) was added to the mixture after 24 h incubation. The mixture was filtered before injection, and HPLC was used to monitor the process 10, 40, and 70 min after adding more peptide.

Antimicrobial Activity of Peptides and the Corresponding Conjugates. The antimicrobial activity of peptides stapled Ac-CGGP9 and the corresponding conjugates against the Gram-negative bacterial strain Klebsiella pneumoniae BL13802 was evaluated within an alamarBlue assay. Bacterial suspensions (100 μL, 5×10⁵ CFU/mL in RPMI) were combined with peptides/conjugates (100 μL, 200 μM peptide concentration) in different wells of a 96-well plate. RPMI buffer alone (200 μL) was added in the plate as a blank control. The bacteria suspension (100 μL) was also incubated with RPMI buffer (100 μL) as untreated control. After incubation (2 h at 37° C. with shaking, 270 rpm), each sample (50 μL) was combined with 2× Luria Broth (LB) medium (50 μL, in triplicates) in the wells of a fresh plate in triplicate. AlamarBlue (10 μL) was added to each well and the plate was covered by a foil and incubated at 37° C. (non-shaking) for 2-3 h. Fluorescence generated from viable bacteria was measured using a plate reader. The reading was taken at 540 nm when the highest fluorescence in the wells was in the range of 6-8×10⁶ relative fluorescent units (RFU). Multiple measurements were taken to make sure the reduction of dye molecules was completed, and the results were read in the range that the fluorescence values were proportional to the concentrations of living bacteria in the well. The average of fluorescence in blank wells was subtracted from the others. The fluorescence was normalized by fluorescence of the average of blanked untreated wells to calculate bacteria survival.

Hemolysis of Stapled Ac-CGGP9 and the Corresponding Conjugates. Human red blood cells (5 mL) were combined with PBS (45 mL, pH 7.4) in a 50 mL centrifuge tube. The solution was mixed by inversion and centrifuged at 500×g for 10 min at room temperature. RBCs were washed twice more as above, then diluted 1:20 (v/v) in RPMI without phenol red and supplemented with low-endotoxin BSA (v/v 1%). Un-colored RPMI buffer was chosen to avoid interference with absorbance measurements. Washed RBCs (180 μL) were combined with peptides/conjugates/controls (20 μL, at peptide concentrations of 2 mM in H₂O) in each of 3 wells of a plate. Uncolored RPMI-BSA media (200 μM) was added on the plate as blank control. H2O (20 μL) and Melittin (20 μL, 80 μM in H₂O) was incubated with washed RBCs (180 μL) as the negative and positive control, respectively. Triton-X (10% in H2O, 20 μL) was incubated with washed RBCs (180 μL) as a control which hemolyzes all RBCs. After incubation (1 h at 37° C.), the plate was centrifuged at 500×g for 5 min. Sample supernatants (100 μL) were transferred to a clear-bottom, black-wall microplate. The absorbance of lysed RBCs was measured at 540 nm using a plate reader. The fluorescence was blank subtracted and normalized by the range from blank to 1% Triton-X 100 (complete hemolysis, 100%) to calculate hemolysis %.

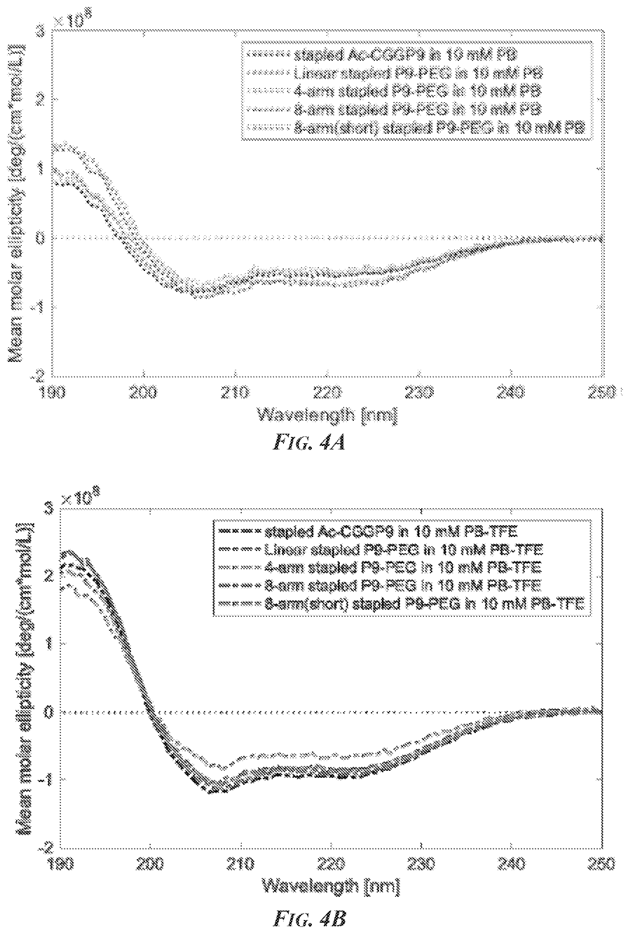

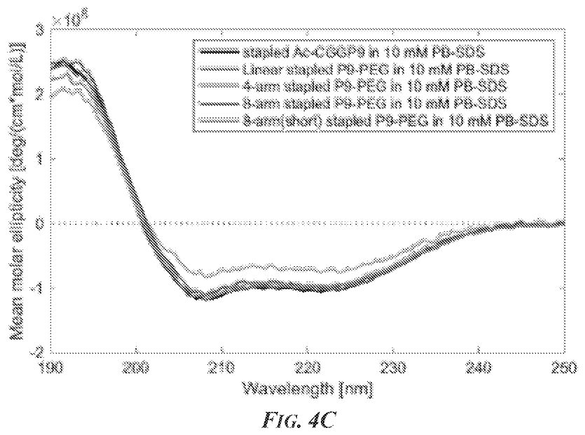

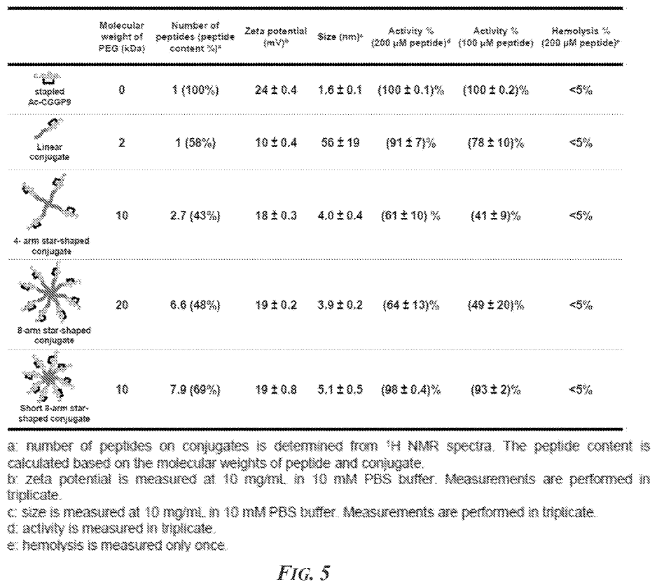

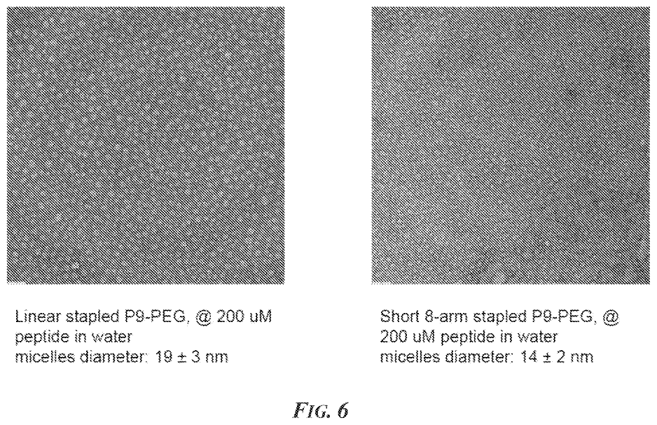

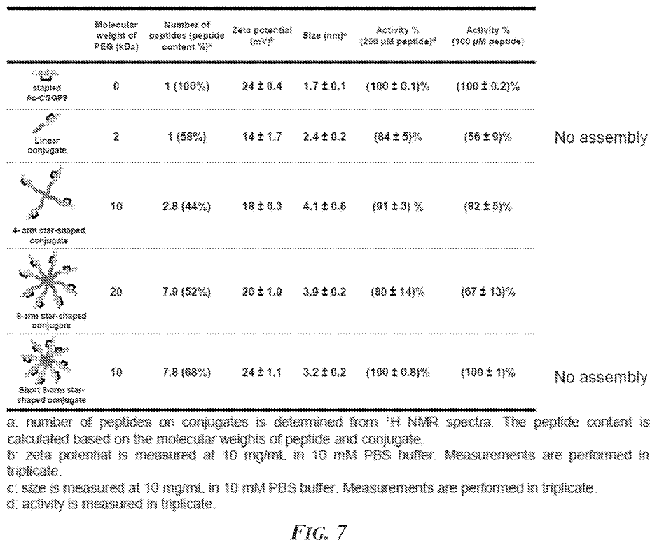

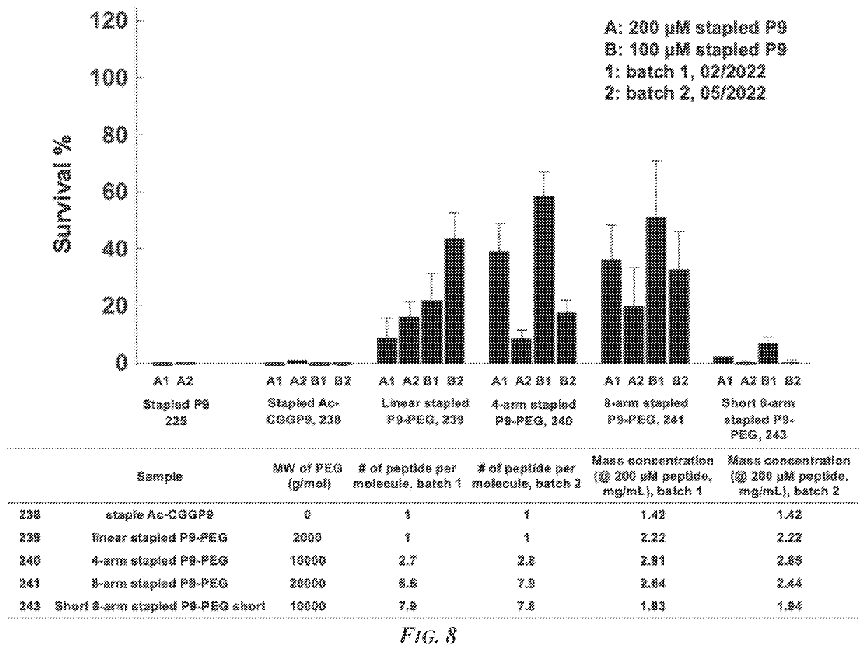

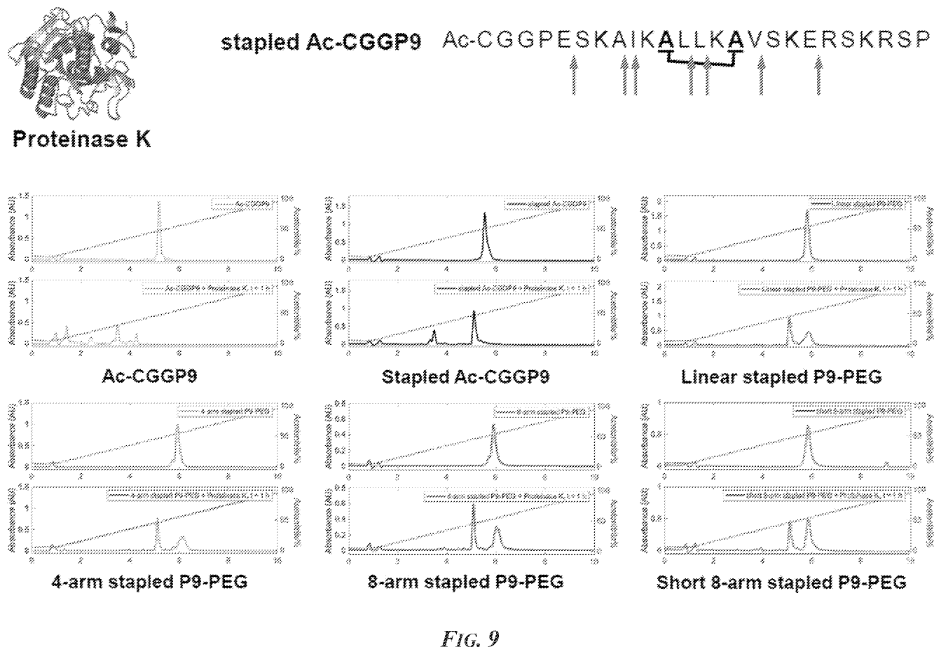

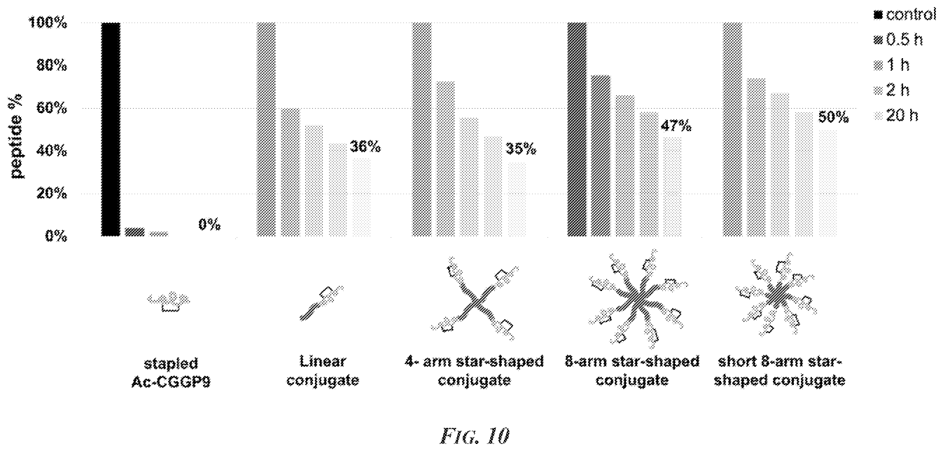

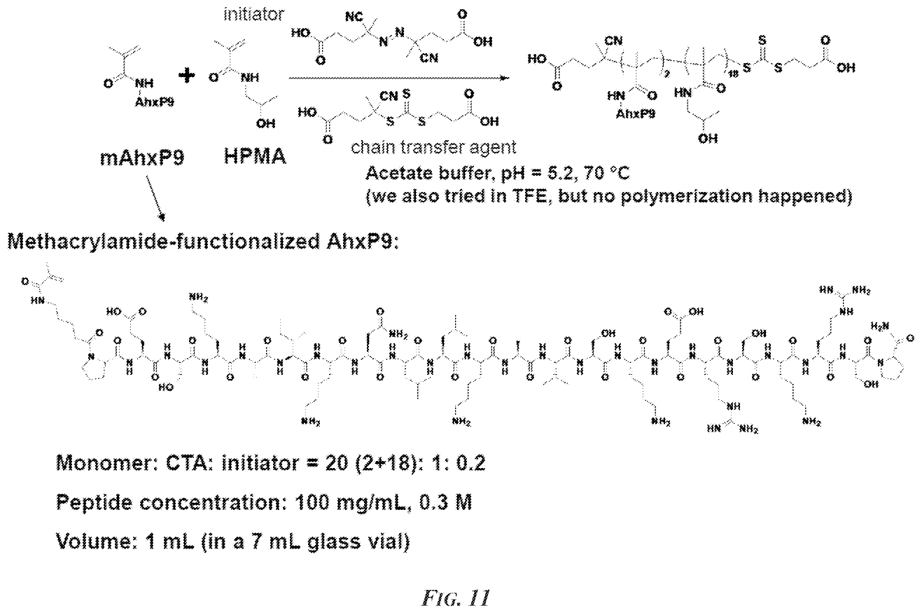

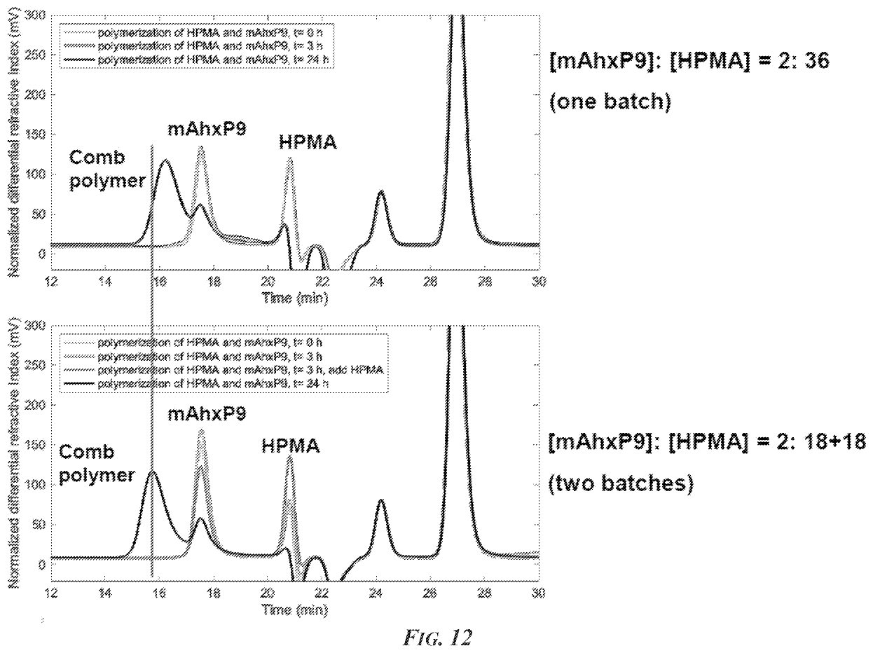

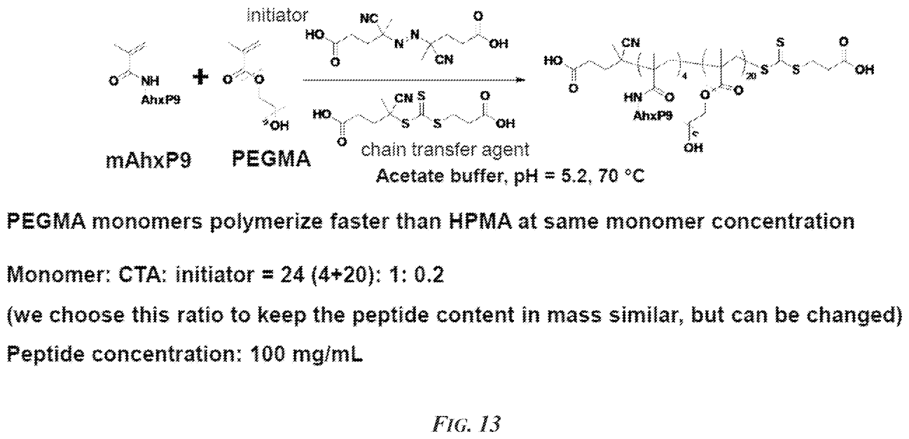

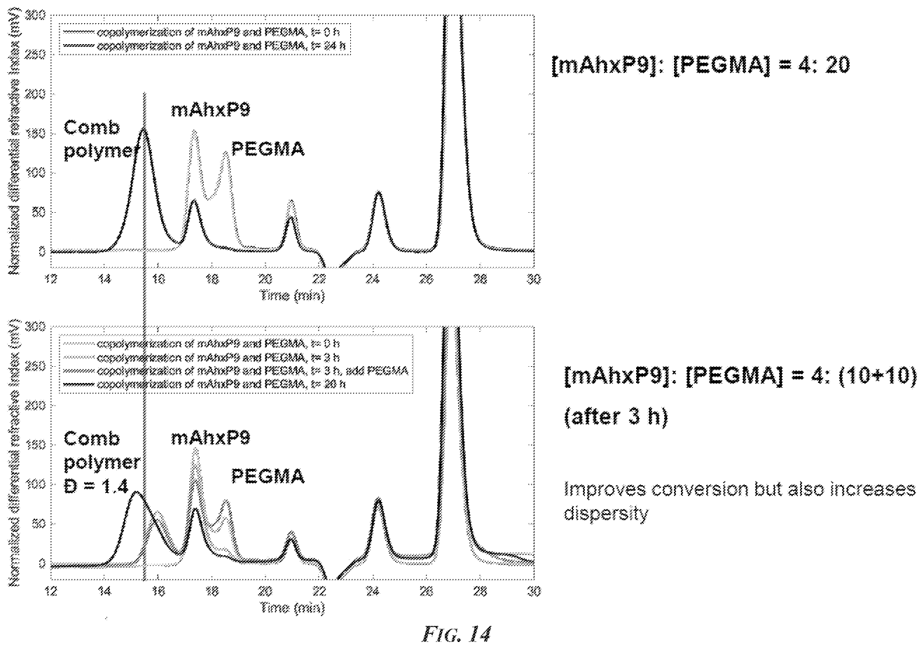

Secondary Structure of Stapled Ac-CGGP9 and the Corresponding Conjugates. Stapled Ac-CGGP9 and the corresponding conjugates were dissolved in 10 mM PB, 50% v/v TFE in 10 mM PB, and 60 mM SDS in 10 mM PB (0.1 mg/mL) (FIGS. 4A-4C). Spectra were obtained from 180-250 nm with a scanning speed of 50 nm/min and integration time of 1 s. Measurements were performed in triplicates and reported as an average. Ellipticity values are converted to mean molar ellipticity by the following equation:

Θₘ=θ_{obs}/(10*1*C_{peptide}*n) EQUATION 1

where θₘ is the mean molar ellipticity (deg/(cm*mol/L)), θ_{obs} is the observed ellipticity (mdeg), 1 is the path length (nm), C_{peptide} is the concentration of peptide (mol/L), and n is the number of amino acid residues per peptide.

While both Ac-CGGP9 and the corresponding conjugates showed only random coil structures in PB, with the staple, both peptide and conjugates showed helical structures in PB (FIG. 4A). The 8-arm conjugates showed slightly higher molar helicity than the others in PB, however, the spectra of these lined up with the other ones in TFE and SDS (FIGS. 4B and 4C), suggesting the difference might not be significant. With the presence of helix-promoting solvent TFE or bacterial membrane mimics SDS, the conjugates all showed higher molar helicity than in PB, indicating that there might be some potential conformational change of the peptide when interacting with bacterial membrane.

Size and Zeta Potential Measurement of Peptides (Stapled Ac-CGGP9 and GGGL8) and the Corresponding Conjugates. Size and zeta potential were measured in 10 mM PBS (pH=7.4) at concentration of 200 μM peptide. A diffusion barrier technique was adapted to protect the electrodes in cuvette and save the usage of samples (https://www.materials-talks.com/diffusionbarrier-method-the-practical-details/). Solution of samples (50 μL) was injected into the U-shaped cuvette with 0.8 mL buffer alone. Measurements were performed in triplicates of same sample.

Activities of Peptides and the Corresponding Conjugates. The activities of peptides (stapled Ac-CGGP9 and GGGL8) and the corresponding conjugates against Gram-negative bacteria K. pneumoniae was measured by alamarBlue analysis. Bacterial suspensions (100 μL, 2.5×105 cfu/mL in RPMI medium) were combined with medium±peptides/controls (at 200 μM peptide concentration) in the wells of a tissue-culture treated plate. Reagent blanks without bacteria were included in each assay. After incubation (2 h at 37° C. with shaking), each sample (50 μL) was combined with 2× Luria Broth (LB) medium (50% v/v, in triplicates) in the wells of a fresh tissue culture-treated plate. AlamarBlue (10 μL) was added to each well and the plate was incubated at 37° C. (protected from light, non-shaking) for 3 hours. Fluorescence generated from viable bacteria was measured using a plate reader. Bacterial survival was calculated as a percentage of the untreated control (bacteria exposed to buffer-alone) according to cfu totals or blank-corrected relative fluorescence units.

Stabilities of Stapled Ac-CGGP9 and the Corresponding Conjugates by HPLC. Stock solutions of Proteinase K (2.5 μM, 0.14 mg/mL, 1×PBS, pH=7.4) and peptide/conjugates (1 mM peptide, 1×PBS, pH=7.4) were prepared right before use. The mass concentration of stapled Ac-CGGP9, linear, 4-arm, 8-arm, and short 8-arm star conjugate stock solutions were 1.78, 2.78, 3.56, 3.04, and 2.42 mg/mL, respectively. Stock solutions were mixed with same volume (0.6 mL for each) and filtered right before characterization. HPLC was used to monitor the degradation over time (0.5 hours, 1 hour, 2 hours, and 20 hours). Control samples were prepared by dilute the stock solution with same volume of 1×PBS. The fractions of peak area were calculated from HPLC by comparing the control samples to the treated ones.

Polymerization of mAhxP9 and PEGMA. The RAFT polymerization was conducted in acetate buffer (pH=5) at 70° C. overnight. Stock solutions of CTA and ACVA were prepared (10 mg/mL in acetate buffer). The PEGMA monomer was run though a beads column to remove the inhibitor.

For one-batch polymerization of peptide and PEGMA, stock solution of CTA (225 μL, 2.2 mg, 0.008 mmol), stock solution of ACVA (40 μL, 0.4 mg, 0.0016 mmol, 0.2 equiv. to CTA), crude peptide monomer mAhxP9 (100 mg, with TFA counter ions, 0.029 mmol, 4 equiv. to CTA), and PEGMA (72 μL, 0.16 mmol, 20 equiv. to CTA) were dissolved in acetate buffer with a final volume of 1 mL in a 7 mL glass shell vial. The monomer solution was de-oxygenated by bubbling through N2 at room temperature for 20 minutes before heating.

For the two-batch polymerization, stock solution of CTA (225 μL, 2.2 mg, 0.008 mmol), stock solution of ACVA (40 μL, 0.4 mg, 0.0016 mmol, 0.2 equiv. to CTA), crude peptide monomer mAhxP9 (100 mg, with TFA counter ions, 0.029 mmol, 2 equiv. to CTA), and PEGMA (36 μL, 0.08 mmol, 10 equiv. to CTA) were dissolved in acetate buffer with a final volume of 0.9 mL in a 7 mL glass shell vial. The monomer solution was deoxygenated by bubbling through N₂ at room temperature for 20 minutes before heating. Another PEGMA solution was prepared (36 μL mg, 0.08 mmol, 10 equiv. to CTA) in 0.1 mL acetate buffer for the second feeding. The solution of PEGMA was degassed for 20 minutes before use. After 3 hours polymerization, the HPMA solution was injected into the polymerization dropwise to prevent the change in temperature.

Samples (10 μL) were taken from 0 to 20 hours during the reaction and diluted with SEC eluent (1 mL) for characterization. SEC samples (50 μL) were injected to the column and the chromatograms were normalized based on the peak of acetate.

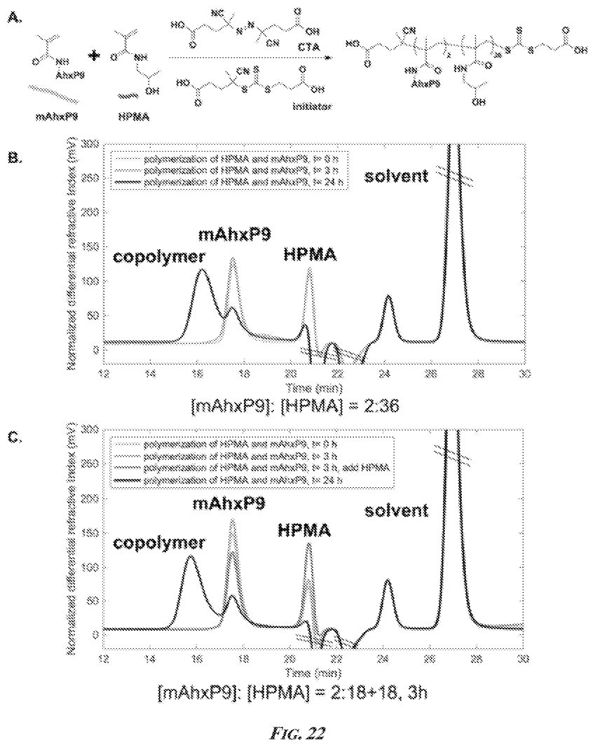

Polymerization of mAhxP9 and HPMA. The RAFT polymerization was conducted in acetate buffer (pH=5) at 70° C. overnight (FIG. 22). Stock solutions of chain transfer agent (4-((((2-Carboxyethyl)thio) carbonothioyl)thio)-4-cyanopentanoic acid; CTA) and initiator (4,4′-Azobis(4-cyanovaleric acid); ACVA) were prepared (10 mg/mL in acetate buffer).

For one-batch polymerization of peptide and HPMA, stock solution of CTA (450 μL, 4.5 mg, 0.015 mmol), stock solution of ACVA (80 μL, 0.8 mg, 0.003 mmol, 0.2 equiv. to CTA), crude peptide monomer mAhxP9 (100 mg, with TFA counter ions, 0.029 mmol, 2 equiv. to CTA), and HPMA (75.2 mg, 0.53 mmol, 36 equiv. to CTA) were dissolved in acetate buffer with a final volume of 1 mL in a 7 mL glass shell vial. The monomer solution was de-oxygenated by bubbling with N₂ (g) at room temperature for 20 minutes before heating.

For the two-batch polymerization, stock solution of CTA (450 μL, 4.5 mg, 0.015 mmol), stock solution of ACVA (80 μL, 0.8 mg, 0.003 mmol, 0.2 equiv. to CTA), crude peptide monomer mAhxP9 (100 mg, with TFA counter ions, 0.029 mmol, 2 equiv. to CTA), and HPMA (37.6 mg, 0.26 mmol, 18 equiv. to CTA) were dissolved in acetate buffer with a final volume of 0.9 mL in a 7 mL glass shell vial. The monomer solution was deoxygenated by bubbling with N₂ (g) at room temperature for 20 min before heating. Another HPMA solution was prepared (37.6 mg, 0.26 mmol, 18 equiv. to CTA) in 0.1 mL acetate buffer for the second feeding. The solution of HPMA was degassed for 20 minutes before use. After 3 hours polymerization, the HPMA solution was injected into the polymerization dropwise to prevent appreciable changes in temperature.

Samples (10 μL) were taken from 0 to 20 hours during the reaction and diluted with SEC eluent (1 mL) for characterization. SEC samples (50 μL) were injected onto the column and the chromatograms were normalized based on the peak of acetate, which elutes between 14-25 minutes.

Similar to the copolymerization of PEGMA and mAHXP9, slowing the polymerization of HPMA by adding the monomers in batches incorporated more peptide monomers. The two-batch copolymerization resulted in copolymers with larger size and a lower peak of unreacted peptide, suggesting a higher conversion of peptide.

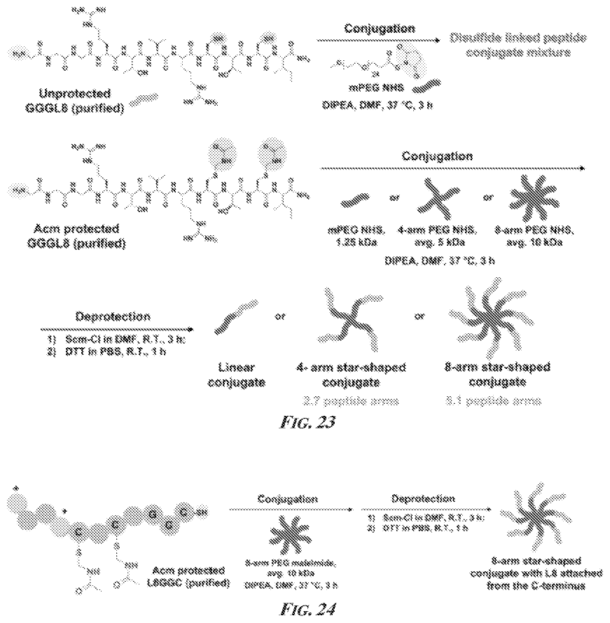

### Conjugation of L8 to Linear and Star-Shaped PEG NHS (See FIG. 23).

Conjugation of GGGL8 to linear PEG NHS. The conjugation reaction of GGGL8 and mPEG NHS was conducted in DMF (2 mL). DMF (10 mL) was de-oxygenated to minimize thiol oxidation by sparging with N₂ (g) for 20 minutes. A stock solution of mPEG NHS (40 mg) in 4 mL degassed DMF was prepared and stored at −20° C. Purified GGGL8 (12 mg, 8 μmol) and DIPEA (0.035 mL, 0.2 mmol) were dissolved in DMF (1 mL) in an 8 mL glass vial. Stock solution of mPEG NHS (1 mL, 10 mg/mL, 8 μmol) was added and the mixture was stirred at 37° C. for 3 hours. The conjugate and unreacted peptide were precipitated into cold ether (8 mL) and dried under vacuum for 30 minutes. To break disulfide bonds formed between the cysteine residues, conjugate mixture (2 mg) was stirred with DTT (5 mg, excess) in 1×PBS (pH=7.4, 1 mL) at room temperature for 1 hour. The mixture was characterized by HPLC.

Conjugation of Acm-protected GGGL8 to linear PEG NHS. Purified peptide GGGL8(Acm)₂ (13 mg, 8 μmol) and DIPEA (0.035 mL, 0.2 mmol) were dissolved in DMF (1 mL) in an 8 mL glass vial for 5 minutes. The peptide solution was mixed with mPEG NHS stock solution (1 mL, 10 mg/mL, 8 μmol) and stirred at 37° C. for 3 hours. Samples (0.05 mL) were taken at different time points, diluted with SEC eluent (0.95 mL) and characterized with SEC to monitor the reaction. The reaction solution was added to cold ether (8 mL) and dried under vacuum for 30 minutes. The dried conjugation mixture was stirred with Scm-Cl (1.5 eq. to Acm, 2.2 μL, 24 μmol) in DCM or DMF (2.5 mL) at room temperature for 3 hours. After precipitating in 10 mL ether, the mixture was dried under vacuum for 30 minutes. To break the disulfide bond on cysteine residues, the mixture was dissolved in 150 mM PBS (2 mL) with DTT (10 mg, excess) and stirred at room temperature for 1 hour. The mixture was purified by preparative HPLC.

Conjugation of Acm-protected GGGL8 to 4- and 8-arm PEG NHS. GGGL8(Acm)₂ (19.5 mg, 12 μmol, 1.5 eq. to NHS group on polymer) was dissolved in DMF (1 mL) with DIPEA (0.035 mL, 0.2 mmol) in an 8 mL glass vial for 5 minutes. The solution of 4-arm PEG NHS (1 mL, 10 mg, 2 μmol) or 8-arm PEG NHS (1 mL, 10 mg, 1 μmol) was added to the peptide solution in four batches in 1 hour and stirred at 37° C. for 3 hours after the mixing. The dried conjugation mixture was dialyzed against DI water with 0.1% TFA with a membrane with MWCO of 1 kDa to remove the unreacted peptide and lyophilized.

The dried conjugates were stirred with Scm-Cl (2 eq. relative to Acm in theory of full conversion, 3.0 μL, 32 μmol) in DMF (2.5 mL) at room temperature for 3 hours. After precipitating in 10 mL ether, the mixture was dried under vacuum for 30 minutes. To break the disulfide bonds among cysteine residues, the mixture was dissolved in 1×PBS (pH=7.4, 2 mL) with DTT (10 mg, excess) and stirred at room temperature for 1 hour. The mixture was purified by dialysis against DI water (×1) and DI water w/0.1% TFA (×2) in a 500 ml beaker with a membrane with MWCO of 2 kDa for 3 hours.

Peptide number determination. The peptide arm numbers on star-shaped conjugates were determined from NMR spectra. Functionalized PEG with different architectures as well as the conjugates were dissolved in deuterated H₂O separately (5 mg/mL) and the samples were scanned 64 times. The protons on polymer core were set as reference peak on the conjugates. The numbers of peptides were calculated as the ratio of the integration of Arg peak on the spectrum of conjugates and the Arg peak on spectrum of the free peptide (Int.=4).

Conjugation of Acm-protected L8(Acm)₂GGC to 8-arm PEG maleimide. The 8-arm PEG maleimide (10 mg, 1 μmol) and L8GGC peptide (19.5 mg, 12 μmol, 1.5 eq. to the maleimide groups) were dissolved in 2 mL 1×PBS (pH=7.4) and stirred at room temperature for 3 hours (FIG. 24). The dried conjugation mixture was dialyzed against DI water with 0.1% TFA with a membrane with MWCO of 1 kDa to remove the unreacted peptide and lyophilized.

The dried conjugates were stirred with Scm-Cl (2 eq. to Acm in theory, 3.0 μL, 32 μmol) in DMF (2.5 mL) at room temperature for 3 hours. After precipitating in 10 mL ether, the mixture was dried under vacuum for 30 minutes. To break the disulfide bond on cysteine residues, the mixture was dissolved in 1×PBS (pH=7.4, 2 mL) with DTT (10 mg, excess) and stirred at room temperature for 1 hour. The mixture was purified by dialysis against DI water (×1) and DI water w/0.1% TFA (×2) in a 500 ml beaker with a membrane with MWCO of 2 kDa for 3 hours.

The peptide arm numbers on star-shaped conjugates were determined from NMR spectra. PEG maleimide with different architectures as well as the conjugates were dissolved in deuterated H₂O separately (5 mg/mL) and the samples were scanned 64 times. The protons on Arg (3.22 ppm, Int.=4) were set as reference peak on the conjugates. The numbers of peptides were calculated as the ratio of the integration of a polymer peak on polymer alone and on conjugates.

Ellman's Assay. To generate a calibration curve, a stock solution of 0.6 mM cysteine monohydrochloride in 100 mM phosphate buffer (pH=8) was prepared first and was diluted to desired concentrations. The stock solutions (100 μL) were then mixed with buffer or Ellman's reagent stock (100 μL) in each well. The absorbance was read on a plate reader at 412 nm with triplicate measurement for one sample. The absorbance was modified by subtracting the buffer with Ellman's reagent.

To measure the available groups on GGGL8 and the linear conjugate, stock solutions of 0.3 mM peptide/linear conjugate (0.6 mM thiol groups) were prepared and diluted to desire concentration (Tables 2-4).

TABLE 2
Recipe of stock solutions of cysteine with
various concentrations in phosphate buffer

| Final thiol | stock thiol | stock cysteine | cysteine | stock | buffer |
|---|---|---|---|---|---|
| conc. (mM) | conc. (mM) | conc. (mM) | (mg) | (mL) | (mL) |
| | 0.6 | 0.6 | 4.22 | 0 | 40 |
| 0.3 | 0.6 | 0.6 | 0 | 5 | 0 |
| 0.24 | 0.48 | 0.48 | 0 | 4 | 1 |
| 0.18 | 0.36 | 0.36 | 0 | 3 | 2 |
| 0.12 | 0.24 | 0.24 | 0 | 2 | 3 |
| 0.06 | 0.12 | 0.12 | 0 | 1 | 4 |
| 0 | 0 | 0 | 0 | 0 | 5 |

TABLE 3
Recipe of stock solutions of GGGL8 with various
concentrations in phosphate buffer

| Final thiol | stock thiol | stock peptide | peptide | stock | buffer |
|---|---|---|---|---|---|
| conc. (mM) | conc. (mM) | conc. (mM) | (mg) | (mL) | (mL) |
| | 0.6 | 0.3 | 4.39 | 0 | 10 |
| 0.3 | 0.6 | 0.3 | 0 | 1.5 | 0 |
| 0.25 | 0.5 | 0.25 | 0 | 1.25 | 0.25 |
| 0.2 | 0.4 | 0.2 | 0 | 1 | 0.5 |
| 0.1 | 0.2 | 0.1 | 0 | 0.5 | 1 |
| 0.05 | 0.1 | 0.05 | 0 | 0.25 | 1.25 |
| 0 | 0 | 0 | 0 | 0 | 1.5 |

TABLE 4
Recipe of stock solutions of linear GGGL8-PEG
with various concentrations in phosphate buffer

| | | Stock | | | |
|---|---|---|---|---|---|
| Final thiol | stock thiol | conjugate | conjugate | stock | buffer |
| conc. (mM) | conc. (mM) | conc. (mM) | (mg) | (mL) | (mL) |
| | 0.6 | 0.3 | 3.74 | 0 | 5 |
| 0.3 | 0.6 | 0.3 | 0 | 1.5 | 0 |
| 0.25 | 0.5 | 0.25 | 0 | 1.25 | 0.25 |
| 0.2 | 0.4 | 0.2 | 0 | 1 | 0.5 |
| 0.1 | 0.2 | 0.1 | 0 | 0.5 | 1 |
| 0.05 | 0.1 | 0.05 | 0 | 0.25 | 1.25 |
| 0 | 0 | 0 | 0 | 0 | 1.5 |

Peptide Purification and Characterization. Preparative-scale reverse-phase high performance liquid chromatography (HPLC) was used to purify stapled Ac-P9 and stapled Ac-CGGP9). The structure was confirmed by matrix-assisted laser desorption ionization (MALDI) time-of-flight (TOF) mass spectrometry and the purity was assessed by analytical HPLC.

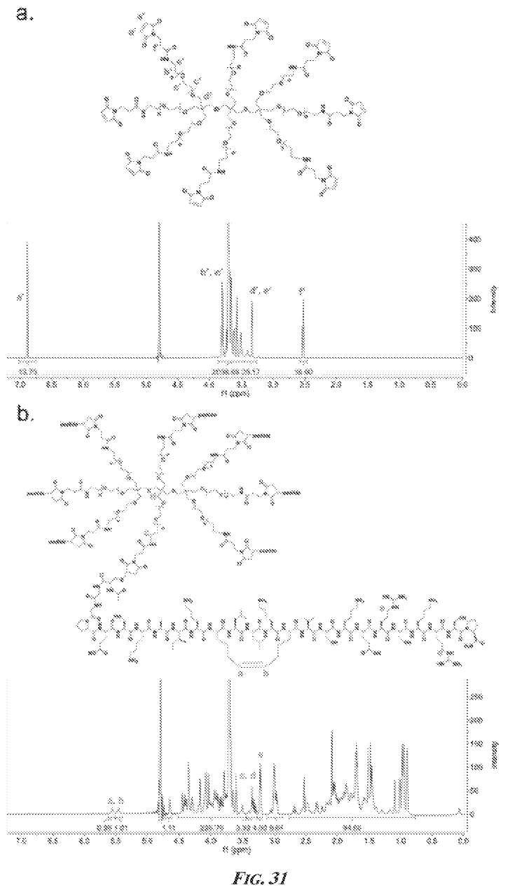

Peptide Arm Number Determination. To determine the average number of peptides “arms” on each conjugate, the ¹H NMR spectra of stapled Ac-CGGP9 and PEG maleimide were compared to their conjugates. The integration of protons on Arg (δH, 3.22 ppm) was set to 4 (peak e), to account for 2 δH on each of the 2 Arg residues per peptide (FIG. 30). For linear PEG maleimide, we set the integration of the 2 methylene protons next to the amide carbonyl (peak f, 2.51 ppm) to 2 for 2H (FIG. 31A). For the linear stapled P9-PEG conjugate, we again set integration of the 4 8H on Arg per peptide (δ=3.22 ppm) to 4, for comparison to peaks c and d (δ=3.37 ppm) that include 5H on the end groups of each polymer. We found peak c and d on the conjugate to integrate to the expected 5H, indicating the successful and quantitative conjugation of one peptide to each polymer chain.

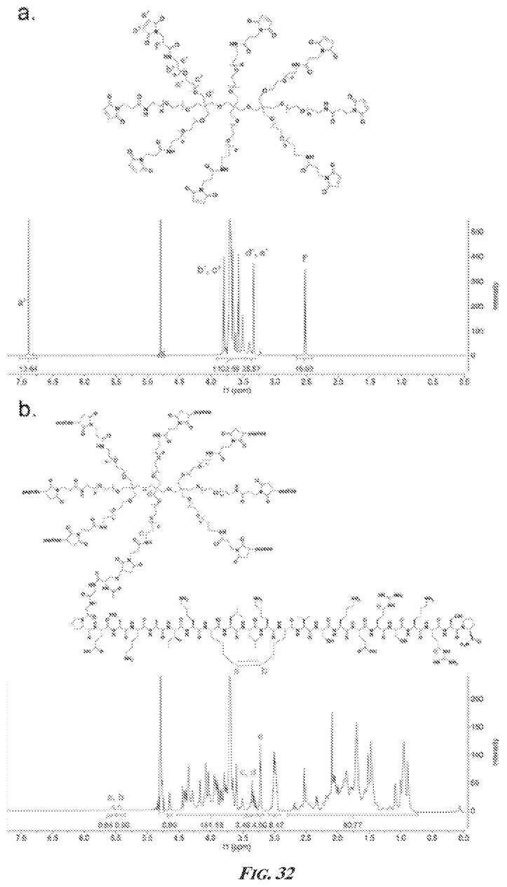

It was determined that the average peptide arm number on the star-shaped conjugates similarly, by setting protons on Arg to 4 and comparing the relative integration of polymer peaks in the spectrum of on polymer alone to those in the conjugate spectra (FIGS. 32-34). The 4-arm, 8-arm, and short 8-arm conjugates have an average of 2.8, 7.4, and 7.3 peptides per molecule, respectively. To improve the conversion of the conjugation to the 4-arm PEG, we tried to use a higher molar ratio of peptide relative to polymer in the conjugation reaction ([thiol group on peptide]: [maleimide on 4-arm PEG]=1.5:1) as well as a new batch of 4-arm PEG maleimide. However, the conversion did not appreciably improve.

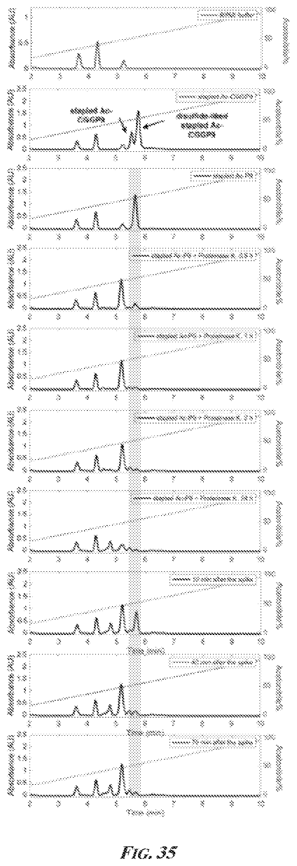

AlamarBlue Assay. The antimicrobial activity of the three independently synthesized batches of peptides and conjugates were tested at peptide concentrations of 100 or 200 μM against Gram-negative Klebsiella Pneumoniae in triplicate. The bacteria survival percent values are plotted in FIG. 35. Peptide and conjugates were active against bacteria at both concentrations, and antimicrobial activity increased with concentration. While linear, 4-arm and 8-arm conjugates varied more between batches, the stapled Ac-P9, linear conjugate, and short 8-arm conjugate showed similar results for three batches at both concentrations.

Hemolysis. Hemolysis caused by the peptide, the cysteine-modified peptide used for conjugation reaction, and the corresponding conjugates was tested at peptide concentration of 200 μM (FIG. 36). Melittin was chosen as a positive control and the results were normalized by the color of lysed red blood cells by 1% Triton X-100. None of the material induced significant hemolysis at this concentration.

Proteolytic Stability of Stapled P9-PEG Conjugates in RPMI. The effect of peptide conjugation on proteolytic stability in the same RPMI buffer used for alamarBlue activity assays was investigated. Interestingly, it was noted the HPLC peak of stapled Ac-CGGP9 split into two, suggesting the cysteine on peptide may oxidize to form disulfide linked peptide. Therefore, the stability of conjugates to stapled Ac-P9, the peptide without CGG linker, was compared since in the conjugate the cysteine is not available for disulfide formation.

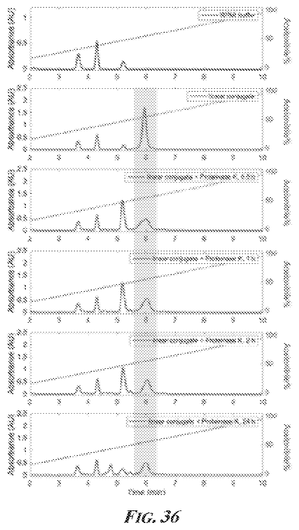

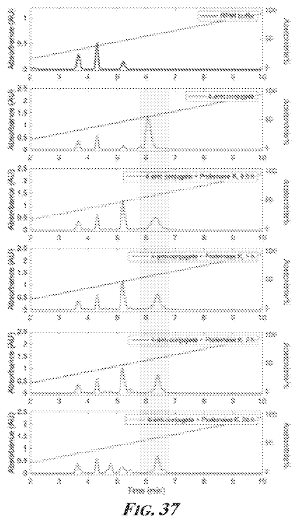

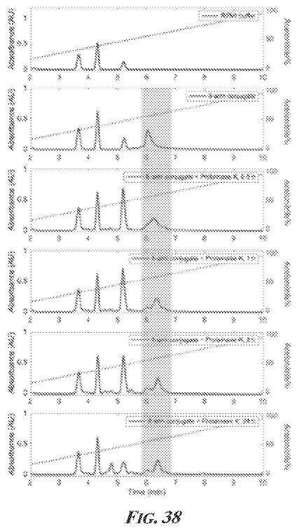

While stapled Ac-P9 fully degraded after 2 hours, the conjugates protected part of the peptide in part after treatment with Proteinase K. To test whether Proteinase K was still functional in the mixtures after 24 hours, more peptide was spiked in the mixture and the mixture was injected onto HPLC after 10, 40, and 70 minutes. After 10 minutes, an increase in the peptide peak was observed, and after 30 minutes, the peptide peak decreased, suggesting Proteinase K was still functional after 24 hours and same condition can be used to monitor the degradation of conjugates. The HPLC traces of Proteinase K treated conjugates and integration of peaks are provided in FIGS. 36-39 and Table 2.

### Example 1

### Synthesis and Purification of Stapled P9-PEG Conjugates

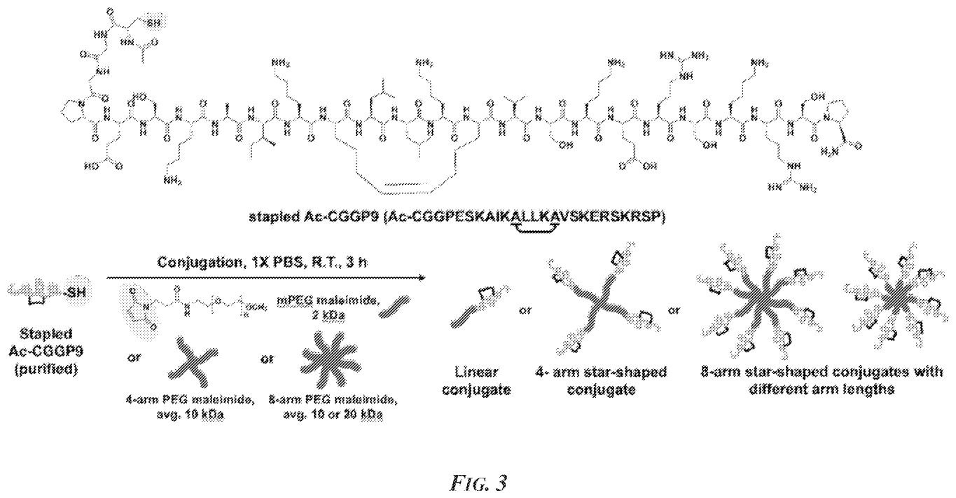

To prepare linear and star-shaped stapled P9-PEG conjugates, we chose a ‘click’-reaction to conjugate peptides with thiol-bearing cysteines at the N-terminus to maleimide-functionalized linear or star-shaped PEG (FIG. 3). While amine-NHS mediated conjugation reactions are widely used in bioconjugation reactions, the thiol-maleimide chemistry was preferred in this case to prevent side reactions involving the lysine amines on the peptide. To reduce steric hinderance, a double-glycine linker was installed next to the cysteine residue. Stapled Ac-CGGP9 was synthesized on a solid phase synthesizer and purified by preparative-scale reverse-phase HPLC to a purity >99%.

The conjugation reactions were performed at room temperature for 3 h with excess peptide (1.1 equiv. to maleimide groups on linear PEG or 1.25 equiv. to maleimide groups on star-shaped PEGs). Monitoring by HPLC, we noticed most of the peptide reacted in the first hour and excess thiol-containing peptide started to form disulfide-linked peptide dimers after 3 h. Thus, before dialysis, the mixtures were treated with DTT to reduce the disulfides and restore the larger size differences between the unreacted peptide and conjugates. A mixture of acidic water and ACN was chosen for dialysis to minimize aggregation and disulfide formation. To avoid the loss of conjugate, HPLC was used to monitor the process and to stop the dialysis when the peak fraction of unreacted peptide in mixture was lower than 5%. Since HPLC traces generated by UV absorption at 214 nm primarily show peptide-containing components in the mixtures, the purified conjugates were then characterized by SEC, on which both polymer and peptide content can be detected by differential refractive index detectors. The peak area on SEC was used to analyze the purity, with all conjugates found to be >95% pure (FIG. 17). The yields of linear, 4-arm, 8-arm, and short 8-arm conjugates were 78%, 75%, 71%, and 70%, respectively.

To determine the average peptide arm numbers on each conjugate, we first compared the peak area of peptide and conjugates on HPLC traces (see Table 5). Interestingly, despite similar amounts of unreacted peptide (or consumption of peptide) in the reaction mixture in HPLC traces, the UV absorbance at 214 nm of the 8-arm conjugate and the short 8-arm conjugate was lower than that of the linear and 4-arm conjugates, indicating the extinction coefficient of those materials may be different. Therefore, the ratio of peak areas of unreacted peptide to conjugate in the mixture may be not suitable for determining the conversion of the conjugation reactions and/or the peptide content on conjugates. Therefore, we used NMR spectra to determine the average number of peptide arms on each conjugate (FIGS. 30-34). We confirmed that the linear conjugate has one peptide arm on each molecule by comparing the integrations of the proton resonance at 3.37 ppm on linear PEG maleimide alone to those on the conjugate. While the 4-arm conjugate had an average around 3 peptide arms, both 8-arm conjugates were almost fully functionalized. We used the average number of peptide arms to estimate the molecular weight of each conjugate for use in calculating conjugate concentration needed to achieve a given peptide concentration to understand the role of polymer architecture in antimicrobial performance and related properties.

### Example 2

### Activity Against Klebsiella pneumoniae

The activity against multi-drug resistant K. pneumoniae was evaluated via alamarBlue assay. Fluorescence generated by the reduced alamarBlue reagent (resazurin) in metabolic active bacteria was measured and normalized as bacteria survival percentages (FIG. 18A). In these assays, we held peptide concentration constant to show how arm number and arm length affect interactions between conjugates and bacteria. Since conjugates with high concentration will kill most bacteria used in the assay, while too low concentration will not yield killing, we chose two peptide concentrations (100 and 200 μM) to obtain a moderate killing to detect differences among conjugates (FIG. 35). The results at 100 μM peptide are shown in FIG. 18C. The measurements were done with three batches of materials that were independently synthesized. The free peptide from all three batches killed most of the bacteria, while the linear conjugate only obtained half of the activity. While we hypothesized increase arm number to increase the local concentration of peptide would enhance the activity, we did not observe significant increase of activity when comparing linear conjugate to the 4- and 8-arm analogues. Several studies show similar findings and suggest that increasing arm number may hinder the pore formation on bacterial membrane, however, in this case assemblies and morphology need to be considered too. Shortening the arm length of the polymer, we found the short 8-arm conjugate exhibited improved activity, consistent with the findings on 4-arm poly(N-isopropylacrylamide-co-amino ethyl acrylamide) with different lengths. We noticed that while the activity of linear, 4-arm, and 8-arm conjugates showed some variations between different batches and different assays, the short 8-arm conjugates consistently show its comparable activity to the stapled Ac-P9, suggesting the high-density structure can be promising. To better explain what lead the differences in activity among the conjugates, how peptides present on polymers needs to be understood by measuring size, surface charge, and morphology of the materials.

### Example 3

### Hemolysis

Hypothesizing attaching AMPs to neutral hydrophilic PEG can better balance activity and toxicity, we measured to hemolysis of peptide and conjugates at same peptide concentration. While previous study showed stapled Ac-P9 did not show significant hemolysis at 50 μM, we increased peptide concentration to 200 μM for both peptide and conjugates to test the toxicity at the concentrations we tested for activity. It is very encouraging that none of the conjugates introduced significant hemolysis at 200 μM peptide. While the free peptides were safe for red blood cells, conjugates having even 8 peptide arms on one molecule behaved similarly, possibly due to the neutral hydrophilic PEG component.

### Example 4

### Stability to Proteinase K

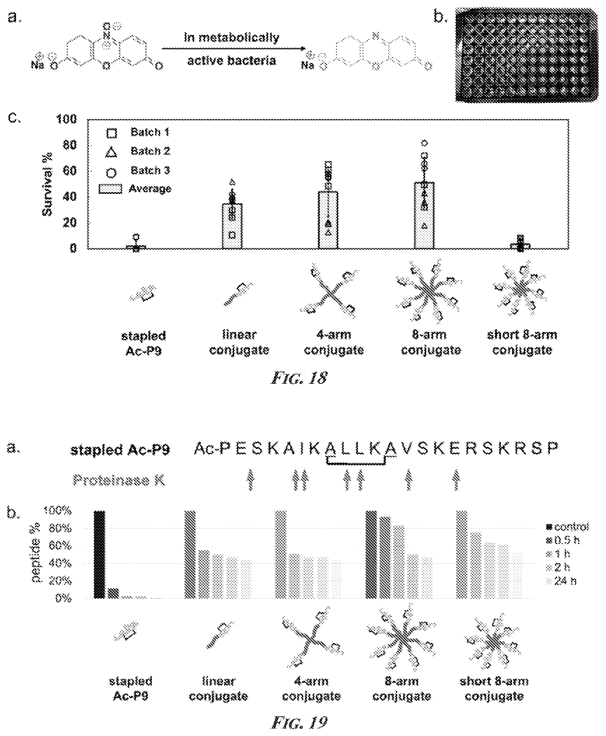

As conjugation to polymers can protect AMPs from enzymatic degradation by shielding peptides from protease, we were interested in how arm number and arm length influence this shielding effect. For these studies, we selected Proteinase K as a model enzyme, as it was predicted to cleave multiple sites on stapled P9 (FIG. 19). To monitor the degradation, we used HPLC to track the fraction of intact peptide by comparing the peptide/conjugate peak area before and after incubation with Proteinase K. We conducted the degradation reactions in same RPMI buffer used for alamarBlue assay to learn if the differences in stability contribute to activity. While the peptide degraded almost fully after a 1 h incubation with Proteinase K, all conjugates showed higher stability (FIGS. 36-39). All conjugates degraded slower after 2 h and retained more than 40% peptide content after 24 h treatment with Proteinase K. In particular, the high-density 8-arm structure preserved ca. 80% peptide conjugated to polymers after 1 h, which was much higher than linear and 4-arm conjugates, possibly due to steric hinderance. To verify that Proteinase K was still active after 24 h, more peptide was added to the mixture. The stapled Ac-P9 peak increased initially after the spike and then decreased, showcasing the Proteinase K was indeed still functional after 24 h after, and therefore, the conjugates exhibited higher stability than the free peptide.

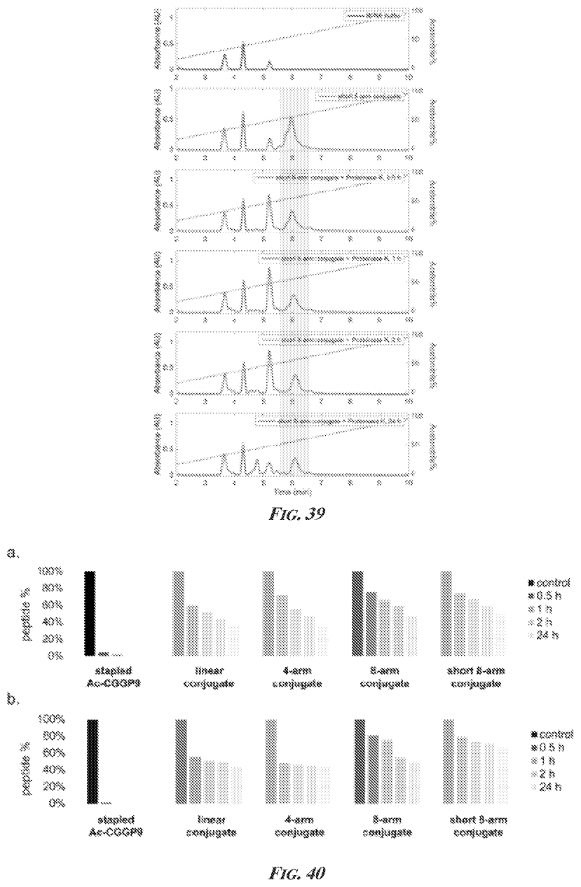

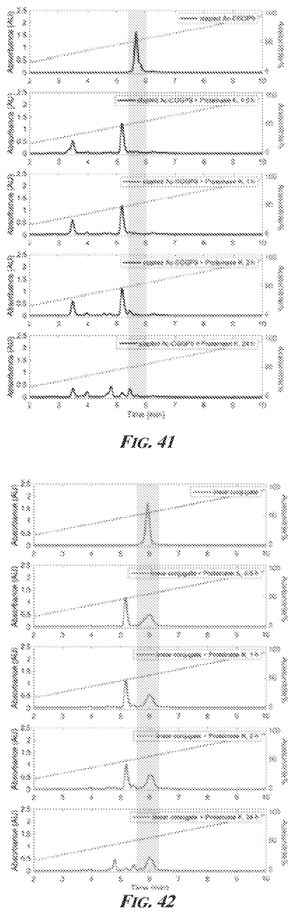

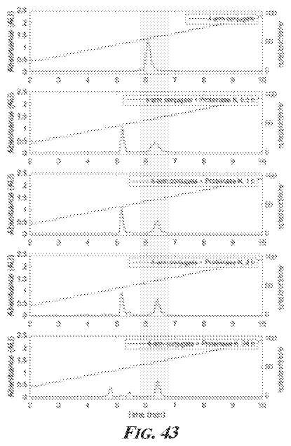

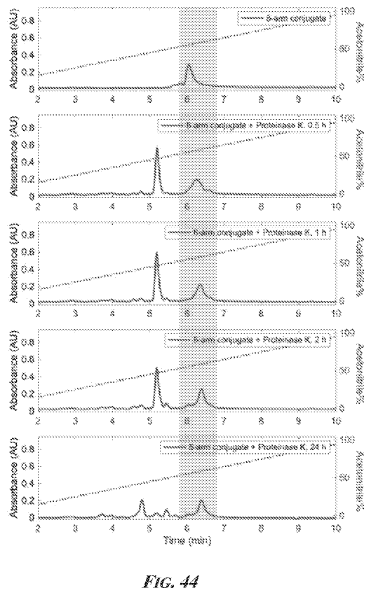

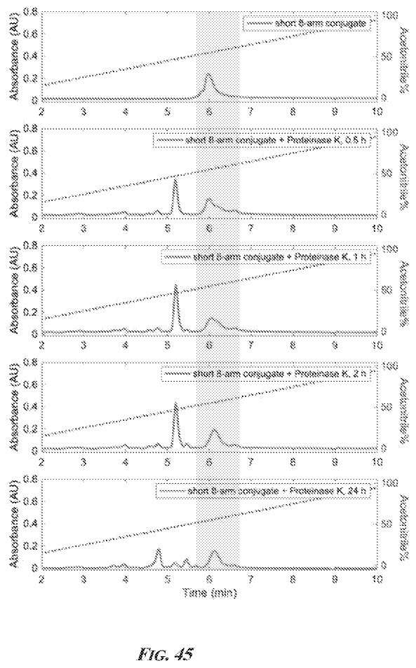

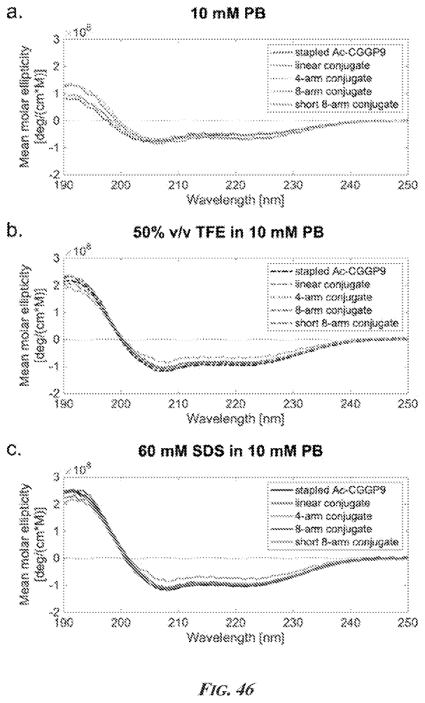

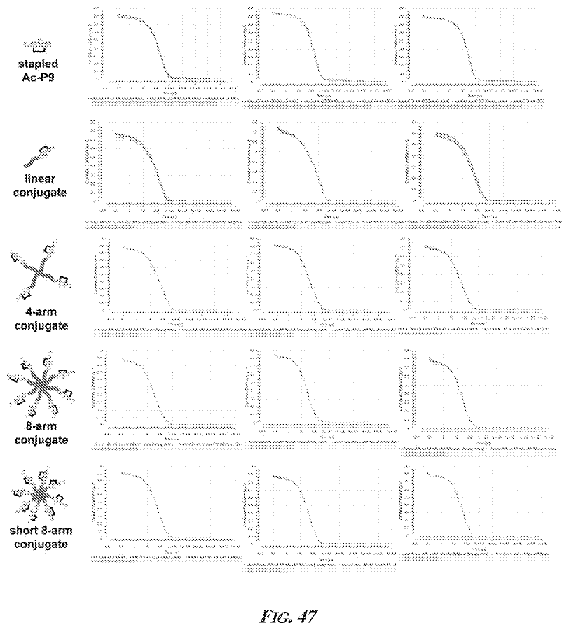

We also incubated peptide/conjugates with Proteinase K in 1×PBS (pH=7.4) to maintain the solution pH since the 25 mM HEPES in RPMI buffer may not prevent the pH shift entirely. While the peptide degraded fully after 30 min treatment, all conjugates showed higher stability, consistent with the findings in RPMI buffer (FIGS. 40-43). Notably, the short 8-arm conjugate that obtained the highest stability among all conjugates preserved more than 60% peptide content after a 24 h incubation.

Degradation of conjugates in both conditions suggested conjugation to polymers hindered Proteinase K from degrading the peptide, and that protection can be enhanced by higher density structures. While the 8-arm conjugates showed slower degradation compared to their linear and 4-arm star analogues, it was not clear whether this protection was due to higher arm numbers on single molecules, or due to supramolecular assembly. Thus, the solution behavior (e.g., secondary structure, size, charge, and morphology) of these molecules were essential to explain the performance results.

### Example 5

### Secondary Structure

Given that the helical structure of stapled Ac-P9 is essential to its antimicrobial activity, thus, we first measured the secondary structure of the conjugates to see if attaching to polymers disrupts the helicity. To better understand how arm number and arm length affect the helical content in different environments, we did the measurement in 3 different solvents (i.e., 10 mM PB, 50% TFE as the hydrophobic cell membrane mimics, and 60 mM SDS as anionic bacterial membrane mimics) and normalize the ellipticity based on peptide concentration and length. All conjugates showed helical structure (2 min values at 206 and 222 nm) similarly as the peptide alone in 10 mM PB, suggesting conjugation to polymer did not disrupt peptide secondary structure. While it is important to have helical structure to be active, the similar molar helicity in different solvents indicated the differences in antimicrobial activity may be due to other properties (e.g., surface charge, size, and morphology).

### Example 6

### Size, Surface Charge, and Morphology

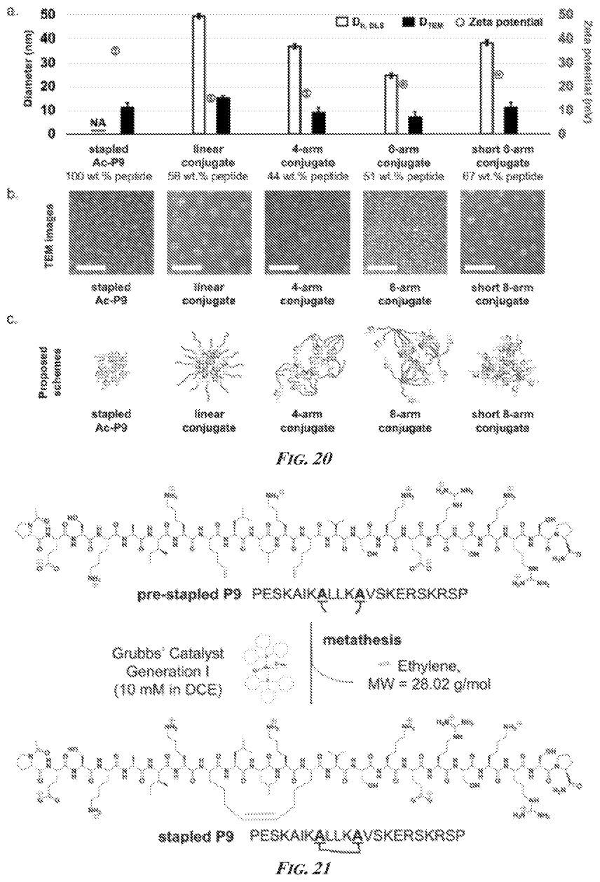

We first measured zeta potential of the peptide and conjugates at constant peptide concentration to gauge the extent of possible electrostatic interactions between peptide/conjugates and bacteria (FIG. 20A). The free peptide showed the highest zeta potential of 35 mV, while the linear conjugate showed just half the zeta potential of the free peptide, consistent with its lower activity compared to peptide alone. While we did not observe significant differences between the zeta potential of the linear and 4-arm conjugates, the 8-arm conjugate showed higher zeta potential compared to the linear conjugates. However, the increase in zeta potential did not benefit its activity, indicating zeta potential might not be the only parameter important to activity. The short 8-arm conjugates showed the highest zeta potential among the conjugates, consistent with its highest activity. We noticed that even the short 8-arm conjugate exhibited comparable activity to the unconjugated peptide, the lower zeta potential indicates the peptide content was shielded by some PEG arms, supporting its higher stability.

We also measured the size of the peptide/conjugates using both DLS and TEM images to study if the conjugates self-assembled in solution (FIGS. 20A and 20B). To validate results fitted by the software on DLS, we fitted the correlograms with decay models including one or multiple size distributions of the particles. We found the results fitted from the single population model was consistent with the Z-average number from DLS, and the results from bimodal distribution were close to the intensity profile generated from the DLS software (Table 4). We suspect the two populations of size indicate both size of the single chain (unimer) and size of the assemblies, results of the latter are larger than the Z-average number and single distribution. Considering that larger structures contribute more than smaller sizes to DLS results, we here use the Z-average number as the hydrodynamic diameters of the particles. Comparing the diameters calculated from DLS (D_{h, DLS}) to the diameters measured from TEM images (D_{TEM}), we found D_{TEM} of the conjugates were all smaller than D_{h, DLS}. This was possibly due to the TEM samples were prepared at dry state, which may lead to decreases in size compared to the measurement in solution via DLS. We found that the mass concentration of unconjugated peptide was only 0.6 mg/mL at 200 μM, which lead to weak scattering when using DLS, resulting in large difference between D_{h, DLS} (150 nm) and D_{TEM} (10 nm) (Table 4), thus the D_{h, DLS} of unconjugated peptide is not applicable.

To understand how peptides present on the conjugates at same peptide concentration, we related their performance to the solution properties including zeta potential, size, and morphology. While the DLS results of the unconjugated peptide may not be trustable due to the low mass concentration, comparing the zeta potential value and TEM images of the peptide to its linear conjugate, we suspect the stapled Ac-P9 was partially hided by the PEG chain due to the hydrophilic, charged amino acid residues, resulting in partially decreased activity but enhancement in stability (FIG. 20C).

The 4-arm conjugate showed smaller size and similar zeta potential to the linear analogue, possibly due to its lower molar concentration and lower peptide content (44%). We hypothesize that the 4-arm conjugates assembled into nanostructure with polymer shell and AMP core, however, the low peptide content resulted in structures where polymer arms can only ‘locked’ some of the peptide content to hinder the interactions with bacteria (FIG. 20C). With the rest of the peptide content on the surface, the morphology of the particles was less structured than the linear analogues. Similarly, the 8-arm conjugate may form nanostructures with peptide cores and PEG shells too. However, different from the 4-arm analogue, the intensity profile of the 8-arm conjugate showed higher fraction of the single chain compared to the other architectures, suggesting the high arm number may prevent the assembly formation. We suspect that the 8-arm conjugates formed less structured aggregates compared to the 4-arm conjugates (FIG. 20C), and the higher zeta potential compared to the 4-arm analogue was attributed from the higher local concentration of peptide. However, the low peptide content (51%) did not lead to significant increase in its antibacterial activity.

Further, the short 8-arm conjugate formed particles having both higher zeta potential and larger size compared to the 8-arm conjugate, possibly due to the short polymer arms can only bridge the aggregated peptide content but unlikely to shield the peptide arms like the 4-arm and longer 8-arm analogs (FIG. 20C). This high-density structure of peptide presented more peptide content on the exterior of the structure but maintain protection from PEG arms, resulting in comparable activity and improved stability to the unconjugated peptide.

Thus, while the hydrophobicity difference between PEG and stapled Ac-P9 can lead to phase separation in solution, we speculate the formation of micelles/aggregates is dependent on both molecular architectures and peptide content. With higher peptide content on the high-density structure, the conjugates may present peptide content in a way to leverage the steric hinderance effect to enhance the stability while maintaining the activity by the multivalency of peptide arms.

TABLE 5
Summary of Raw HPLC Peak Area Data of Peptide/Conjugate Treated
by Proteinase K in RPMI Buffer After 0.5, 1, 2, and 24 hours

| Peak area on HPLC | control | after 0.5 h | after 1 h | after 2 h | after 24 h |
|---|---|---|---|---|---|
| stapled Ac-P9 | 12404892 | 1459303 | 338152 | 316184 | 147487 |
| linear conjugate | 18591375 | 10240577 | 9217317 | 8806783 | 8241829 |
| 4-arm conjugate | 18801136 | 9520151 | 8921037 | 8927364 | 8381981 |
| 8-arm conjugate | 4934828 | 4554038 | 4078143 | 2458611 | 2317713 |
| short 8-arm conjugate | 9811759 | 7352429 | 6239023 | 6089020 | 5124387 |

TABLE 6
Summary of Raw HPLC Peak Area Data of Peptide/Conjugate Treated
with Proteinase K in 1X PBS After 0.5, 1, 2, and 24 Hours

| Peak area on HPLC | control | after 0.5 h | after 1 h | after 2 h | after 24 h |
|---|---|---|---|---|---|
| stapled Ac-CGGP9 | 14565457 | 245330 | 0 | 0 | 0 |
| linear conjugate | 17325483 | 9683812 | 8754412 | 8486414 | 7625727 |
| 4-arm conjugate | 15842706 | 7652565 | 7430065 | 7245074 | 6995785 |
| 8-arm conjugate | 4566786 | 3711678 | 3443403 | 2502010 | 2249326 |
| short 8-arm conjugate | 4036048 | 3192181 | 2981129 | 2898030 | 2724360 |

TABLE 8
Table S3 Zeta Potential and Size (Diameter) of Stapled Ac-P9 and its Conjugates

| | | stapled | linear | 4-arm | 8-arm | short 8-arm |
|---|---|---|---|---|---|---|
| | | Ac-P9 | conjugate | conjugate | conjugate | conjugate |
| Zeta potential | 400 μM | 39 ± 2.7 | 15 ± 0.8 | 20 ± 0.3 | 23 ± 1.0 | 29 ± 0.6 |
| (mV) | 200 μM | 35 ± 1.2 | 15 ± 1.1 | 17 ± 1.1 | 21 ± 0.5 | 25 ± 0.2 |
| | 100 μM | — | 11 ± 2.4 | 17 ± 0.8 | 19 ± 0.5 | 25 ± 1.3 |
| Diameter, DLS | 400 μM | 140.4 ± 15.6 | 88.9 ± 3.8 | 45.7 ± 1.3 | 27.6 ± 0.2 | 54.0 ± 1.7 |
| (nm) | 200 μM | 145.6 ± 4.3 | 49.2 ± 1.0 | 36.9 ± 1.2 | 24.6 ± 0.3 | 38.3 ± 0.2 |
| | 100 μM | 294.3 ± 15.8 | 45.3 ± 4.6 | 37.8 ± 2.3 | 22.5 ± 0.4 | 32.8 ± 0.3 |
| Diameter, fitted, | 200 μM | — | 54.4 | 43.6 | 28.8 | 43.8 |
| monomodal (nm) | | | | | | |
| Diameter, fitted, | 200 μM | — | 3.3, 92.7 | 8.2, 94.1 | 10.5, 79.9 | 9.8, 76.7 |
| bimodal (nm) | | | | | | |
| Diameter, TEM (nm) | 200 μM | 11 ± 2.2 | 15 ± 1.0 | 9 ± 2.1 | 7 ± 2.4 | 11 ± 2.3 |

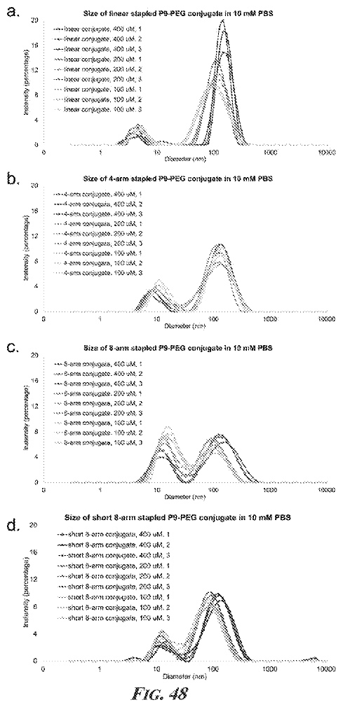

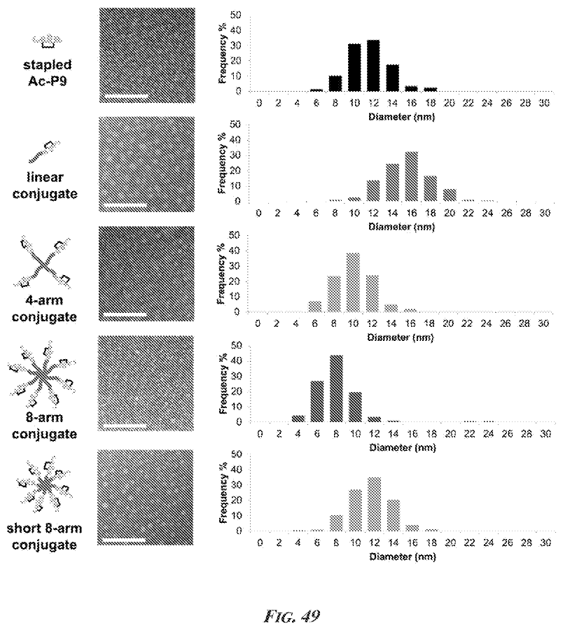

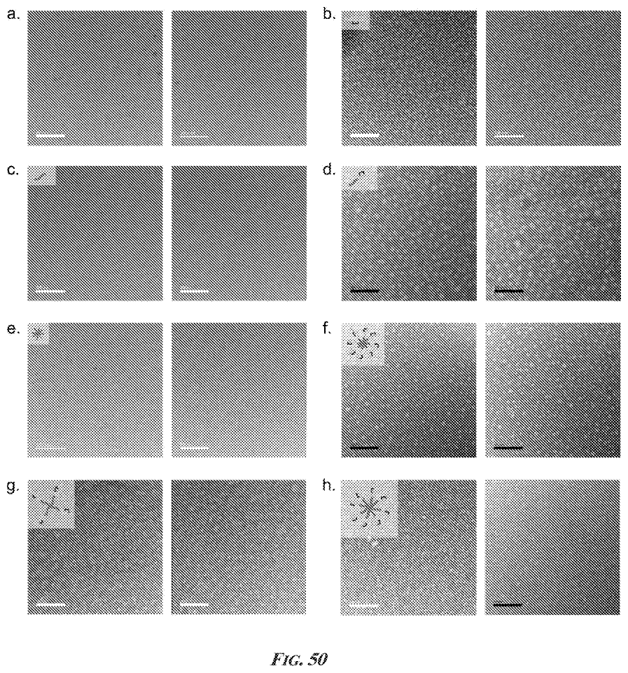

Measurements were taken at 100, 200, and 400 μM peptide in 10 mM PBS in triplicates. For stapled Ac-P9, the zeta potential measurement was limited by the low concentration at 100 μM (0.3 mg/mL). Z-average values are reported as the hydrodynamic diameter of the materials. The correlograms were fitted by two models to calculate the diameter populations. TEM images were taken at 200 μM peptide concentration and were analyzed by ImageJ and the histograms were obtained from Excel by counting 200 samples. Histograms are listed in FIG. 49.

Measurements were taken at 100, 200, and 400 μM peptide in 10 mM PBS in triplicates. For stapled Ac-P9, the zeta potential measurement was limited by the low concentration at 100 μM (0.3 mg/mL). Z-average values are reported as the hydrodynamic diameter of the materials. The correlograms were fitted by two models to calculate the diameter populations. TEM images were taken at 200 μM peptide concentration and were analyzed by ImageJ and the histograms were obtained from Excel by counting 200 samples. Histograms are listed in FIG. 49.

### Discussion of the Examples

Antimicrobial peptides (AMPs) are promising alternatives to conventional antibiotics in treating drug-resistant bacteria, yet they are limited by toxicity to mammalian cells and instability in biological environments. Previous studies showcase conjugating peptide to non-linear neutral, hydrophilic polymers provide opportunities to better balance activity and toxicity than cationic polymers, however, not much polyethylene glycol (PEG) involved. As disclosed herein, the cytokine-derived AMP, stapled Ac-P9, was conjugated to linear and star-shaped PEG with various arm numbers and arm lengths to determine the role of molecular architecture on the solution properties and functions. It was found that all conjugates showed enhanced proteolytic stability, while the short 8-arm conjugate maintained the antimicrobial activity compared to the unconjugated peptide. To connect the molecular architecture to the performance, the solution properties (e.g., secondary structure, zeta potential, size, and morphology) of the conjugates were measured. Linear, 4-arm, and 8-arm conjugates 2-2.5 kDa PEG arms formed nanoscale structures in solution with lower zeta potential relative to the unconjugated AMP, suggesting a lower density of cationic AMP available at the surface. While clear trends in antimicrobial activity as a function of arm number were not observed, potentially due to variations in peptide content and solution assembly among these conjugates, the 8-arm conjugate conferred the highest proteolytic stability in the first 1 hour incubation with enzyme. Reducing the arm length of the 8-arm conjugate to 1.25 kDa per arm could lead the molecules to assemble in a different way than the other architectures, resulting in increased antimicrobial activity while maintaining high proteolytic stability. The performance was attributed to its high peptide content and high peptide density, resulting assembled nanostructure with high zeta potential among all conjugates. Considered together, while enhancing proteolytic stability often comes with the cost of lower antimicrobial activity, these results suggested that displaying AMPs at sufficiently high density on a neutral non-linear polymer could strike a nice balance to achieve both high antimicrobial activity and stability.

As disclosed herein, we prepared a series of conjugates of stapled Ac-P9 with various arm number and arm length. We chose linear, 4-, and 8-arm PEG maleimide as those are commercially available, while maleimide-functionalized PEG with higher arm number would require grafting-from or grafting-through approaches to increase the complexity in synthesis. Previous studies show conjugating AMPs to PEG with similar molecular weight better retain their activity than longer PEG, and in this case, we selected linear and star-shaped PEG with similar length based on the peptide's MW. While shorter linear conjugates with higher activity often show lower stability compared to longer ones, here, we showed that the shorter star-shaped conjugates would obtain both higher activity and stability, which can be promising for delivering other AMPs.

For the synthesis of peptide and conjugates, we adapted thiol-maleimide chemistry due to the presence of amin-containing lysine residues, while for AMPs with both lysine and cysteine residues, other strategies will be needed for conjugation reactions. We noticed some challenges during dialysis potentially due to the small size differences between peptide and conjugates, and the 8-arm conjugates that need longer dialysis and longer monitoring process had slightly lower yield.

To connect molecular design to antimicrobial performance, we measured properties that are important to activity of stapled Ac-P9 alone including secondary structure, size, zeta potential, and morphology at similar peptide concentration. The unconjugated stapled Ac-P9 showed high antimicrobial activity but low proteolytic stability, the conjugates with neutral PEG formed particles with lower zeta potential to shielded peptide content to improve the stability. With the protection from PEG chains (ca. 50% in mass), the linear, 4-arm, and 8-arm conjugates all showed decreased activity compared to the unconjugated peptide at same peptide concentration. It is encouraging that shortening the arm length to increase the peptide content to 70% yielded particles with highest zeta potential among the conjugates to increase the activity, while the assemblies masked the peptide content in the micelles to maintain the stability.

Taken together, we found varying the peptide density and peptide content adjusted how to present the peptide on these nanostructures to balance their stability and activity. The short 8-arm conjugates having the highest peptide density and peptide content (%) formed particles to retain comparable antibacterial activity and enhanced proteolytic stability to the unconjugated peptide. We anticipate that high-density structure with high peptide content is promising in designing self-assembled AMP-PEG conjugates to balance their performance.

### REFERENCES

All references listed below and in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1993) Basic local alignment search tool. J Mol Biol 215:403-410.
Arranz-Gibert et al. (2018) Immunosilencing peptides by stereochemical inversion and sequence reversal: retro-D-peptides. Sci Rep 8(1):6446.
Ausubel et al. (2002) Short Protocols in Molecular Biology, Fifth ed. Wiley, New York, New York, United States of America.
Ausubel et al. (2003) Current Protocols in Molecular Biology. John Wylie & Sons, Inc, New York, New York, United States of America.
Bila et al. (2019) Engineering a stable future for DNA-origami as a biomaterial. Biomater Sci 7(2):532-541.
Bird et al. (1988) Single-chain antigen-binding proteins. Science 242:423-426.
Blum et al. (2014) Peptides displayed as high density brush polymers resist proteolysis and retain bioactivity. J Am Chem Soc 136(43):15422-15437.
Borro & Malmsten (2019) Complexation between antimicrobial peptides and polyelectrolytes. Adv Colloid Interface Sci 270:251-260.
Bowie et al. (1990) Deciphering the message in protein sequences: tolerance to amino acid substitutions. Science 247:1306-1310.
Chang et al. (2015) Functional Sulfobetaine Polymers: Synthesis and Salt-Responsive Stabilization of Oil-in-Water Droplets. Macromolecules 48(21):7843-7850.
Chang et al. (2017) Sequence and entropy-based control of complex coacervates. Nat Commun 8(1):1273.
Chiti & Dobson (2006) Protein misfolding, functional amyloid, and human disease. Annu Rev Biochem 75:333-366.
Chou & Fasman (1974) Prediction of protein Conformation. Biochemistry 13:222-245.
Chou & Fasman (1978) Empirical predictions of protein conformation. Ann Rev Biochem 47:251-276.
Chou & Fasman (1979) Prediction of beta-turns. Biophys J 26:367-384.
Chu et al. (2015) Multivalent display of pendant pro-apoptotic peptides increases cytotoxic activity. J Control Release 205:155-161.
Collier & Segura (2011) Evolving the use of peptides as components of biomaterials. Biomaterials 32(18):4198-4204.
Crawford et al. (2017) CXC Chemokines Exhibit Bactericidal Activity against Multidrug-Resistant Gram-Negative Pathogens. mBio 8(6):e01549-17.
Cui et al. (2021) Molecular engineering of antimicrobial peptide (AMP)-polymer conjugates. Biomater Sci 9(15):5069-5091.
Dayhoff et al. (1978) in Atlas of Protein Sequence and Structure 1978, National Biomedical Research Foundation, Washington D.C., United States of America.
Dong et al. (2019) Experiments and Simulations of Complex Sugar-Based Coil-Brush Block Polymer Nanoassemblies in Aqueous Solution. ACS Nano 13(5):5147-5162.
Dong et al. (2020) Degradable sugar-based magnetic hybrid nanoparticles for recovery of crude oil from aqueous environments. Polymer Chemistry 11(30):4895-4903.
Elder et al. (2011) Understanding the effect of polylysine architecture on DNA binding using molecular dynamics simulations. Biomacromolecules 12(11):3870-3879.
Fan et al. (2017) Multi-responsive polypeptide hydrogels derived from N-carboxyanhydride terpolymerizations for delivery of nonsteroidal anti-inflammatory drugs. Org Biomol Chem 15(24):5145-5154.
Foster et al. (2017) The Diverse Roles of Hydrogel Mechanics in Injectable Stem Cell Transplantation. Curr Opin Chem Eng 15:15-23.
Ghobadi et al. (2016) Dispersing Zwitterions into Comb Polymers for Nonviral Transfection: Experiments and Molecular Simulation. Biomacromolecules 17(2):546-557.
Grewal et al. (2021) User-defined, temporal presentation of bioactive molecules on hydrogel substrates using supramolecular coiled coil complexes. Biomater Sci 9(12):4374-4387.
Griffin et al. (2021) Activating an adaptive immune response from a hydrogel scaffold imparts regenerative wound healing. Nat Mater 20(4):560-569.
Harlow & Lane (1988) Antibodies: A Laboratory Manual. Cold Spring Harbor Laboratory Press, Cold Spring Harbor, New York, United States of America.
Harlow & Lane (1999) Using Antibodies: A Laboratory Manual. Cold Spring Harbor Laboratory Press, Cold Spring Harbor, New York, United States of America.
Henikoff & Henikoff (1992) Amino acid substitution matrices from protein blocks. Proc Natl Acad Sci USA 89:10915-10919.
Houston et al. (1988) Protein engineering of antibody binding sites: Recovery of specific activity in an anti-digoxin single-chain Fv analogue produced in Escherichia coli. Proc Natl Acad Sci USA 85(16):5879-5883.
Imura et al. (2007) Action mechanism of tachyplesin I and effects of PEGylation. Biochim Biophys Acta 1768(5):1160-1169.
Jiang et al. (2019) Multivalent Polymer-Peptide Conjugates-A General Platform for Inhibiting Amyloid Beta Peptide Aggregation. ACS Macro Lett 8(10):1365-1371.
Johnson & Overington (1993) A structural basis for sequence comparisons. An evaluation of scoring methodologies. J Mol Biol 233:716-738.
Johnson et al. (2010) Synthesis of statistical copolymers containing multiple functional peptides for nucleic Acid delivery. Biomacromolecules 11(11):3007-3013.
Johnson et al. (2011) HPMA-oligolysine copolymers for gene delivery: optimization of peptide length and polymer molecular weight. J Control Release 155(2):303-311.
Johnston et al. (2017) The effect of comb architecture on complex coacervation. Org Biomol Chem 15(36):7630-7642.
Joralemon et al. (2010) PEGylated polymers for medicine: from conjugation to self-assembled systems. Chem Commun (Camb) 46(9):1377-1393.
Kalasin et al. (2017) Adsorbed Polyzwitterion Copolymer Layers Designed for Protein Repellency and Interfacial Retention. Langmuir 33(47):13708-13717.
Karlin & Altschul (1993) Applications and statistics for multiple high-scoring segments in molecular sequences. Proc Natl Acad Sci USA 90(12):5873-5877.
Kizilbey et al. (2018) An In vivo study: Adjuvant activity of poly-n-vinyl-2-pyrrolidone-co-acrylic acid on immune responses against Melanoma synthetic peptide. Bioengineered 9(1):134-143.
Kopecek & Yang (2020) Polymer nanomedicines. Adv Drug Deliv Rev 156:40-64.
Kuenen et al. (2021) Buffering effects on the solution behavior and hydrolytic degradation of poly(β-amino ester)s. Journal of Polymer Science 59(19):2212-2221.
Kuenen et al. (2023) Net anionic poly(O-amino ester)s: synthesis, pH-dependent behavior, and complexation with cationic cargo. Polymer Chemistry 14:421-431.
Kwon et al. (2014) Poly-dipeptides encoded by the C9orf72 repeats bind nucleoli, impede RNA biogenesis, and kill cells. Science 345(6201):1139-1145.
Kyte & Doolittle (1982) A simple method for displaying the hydropathic character of a protein. J Mol Biol 157:105-132.
Letteri et al. (2017) Forming Sticky Droplets from Slippery Polymer Zwitterions. Adv Mater 29(38):1702921.
Li et al. (2016) Roles of d-Amino Acids on the Bioactivity of Host Defense Peptides. Int J Mol Sci 17(7):1023.
Li et al. (2019) Functional, Degradable Zwitterionic Polyphosphoesters as Biocompatible Coating Materials for Metal Nanostructures. Langmuir 35(5):1503-1512.
Lin et al. (2020) Erythrocyte-Membrane-Camouflaged Nanocarriers with Tunable Paclitaxel Release Kinetics via Macromolecular Stereocomplexation. ACS Materials Letters 2(6):595-601.
Lytle et al. (2019) Designing Electrostatic Interactions via Polyelectrolyte Monomer Sequence. ACS Cent Sci 5(4):709-718.
Mori et al. (2013) The C9orf72 GGGGCC Repeat Is Translated into Aggregating Dipeptide-Repeat Proteins in FTLD/ALS. Science 339(6125):1335-1338.
Needleman & Wunsch (1970) A general method applicable to the search for similarities in the amino acid sequence of two proteins. J Mol Biol 48(3):443-453.
Ngambenjawong & Pun (2017) Multivalent polymers displaying M2 macrophage-targeting peptides improve target binding avidity and serum stability. ACS Biomater Sci Eng 3(9):2050-2053.
Nguyen et al. (2020) Antibody Therapy Targeting RAN Proteins Rescues C9 ALS/FTD Phenotypes in C9orf72 Mouse Model. Neuron 105(4):645-662 eli.
Nielsen et al. (1991) Sequence-selective recognition of DNA by strand displacement with a thymine-substituted polyamide. Science 254:1497-1500.
Parelkar et al. (2011) Reconfiguring polylysine architectures for controlling polyplex binding and non-viral transfection. Biomaterials 32(9):2432-2444.
Parelkar et al. (2014) Polymer-peptide delivery platforms: effect of oligopeptide orientation on polymer-based DNA delivery. Biomacromolecules 15(4):1328-1336.
PCT International Patent Application Publication No. WO 2020/023670.
Pearson & Lipman (1988) Improved tools for biological sequence comparison. Proc Natl Acad Sci USA 85:2444-2448.
Perry et al. (2015) Chirality-selected phase behaviour in ionic polypeptide complexes. Nat Commun 6:6052.
Priftis et al. (2015) Self-assembly of alpha-helical polypeptides driven by complex coacervation. Angew Chem Int Ed Engl 54(38):11128-11132.
Ran et al. (2020) Allstage precisional glioma targeted therapy enabled by a well-designed D-peptide. Theranostics 10(9):4073-4087.
Raveendran et al. (2020) Estrone-Decorated Polyion Complex Micelles for Targeted Melittin Delivery to Hormone-Responsive Breast Cancer Cells. Biomacromolecules 21(3):1222-1233.
Remington et al. (1975) Remington's Pharmaceutical Sciences, 15th ed. , Mack Pub. Co., Easton, Pennsylvania, United States of America.
Santa Chalarca et al. (2018) Building Supracolloidal Fibers from Zwitterion-Stabilized Adhesive Emulsions. Adv Funct Mater 28(45):1804325.
Sela & Zisman (1997) Different roles of D-amino acids in immune phenomena. FASEB J 11(6):449-456.
Shi et al. (2017) Toxic PRn poly-dipeptides encoded by the C9orf72 repeat expansion block nuclear import and export. Proc Natl Acad Sci USA 114(7):E1111-E1117.
Sing & Perry (2020) Recent progress in the science of complex coacervation. Soft Matter 16(12):2885-2914.
Slager & Domb (2003) Biopolymer stereocomplexes. Advanced Drug Delivery Reviews 55(4):549-583.
Smith & Waterman (1981) Local homologies algorithm to align sequences. Adv Appl Math 2:482-489.
Smith et al. (2020) Polymer-Peptide Conjugates Convert Amyloid into Protein Nanobundles through Fragmentation and Lateral Association. ACS Appl Nano Mater 3(2):937-945.
Song et al. (2017a) Crystallization-driven assembly of fully degradable, natural product-based poly(1-lactide)-block-poly(α-d-glucose carbonate)s in aqueous solution. Polymer 122:270-279.
Song et al. (2017b) Polymer-Peptide Conjugates Disassemble Amyloid beta Fibrils in a Molecular-Weight Dependent Manner. J Am Chem Soc 139(12):4298-4301.
Song et al. (2021) Morphologic Design of Silver-Bearing Sugar-Based Polymer Nanoparticles for Uroepithelial Cell Binding and Antimicrobial Delivery. Nano Lett 21(12):4990-4998.
Taylor et al. (2016) Decoding ALS: from genes to mechanism. Nature 539(7628):197-206.
Tsuji (2016) Poly(lactic acid) stereocomplexes: A decade of progress. Adv Drug Deliv Rev 107:97-135.
U.S. Pat. Nos. 4,554,101; 5,034,506.
Wang et al. (2019) Polyion complexes of a cationic antimicrobial peptide as a potential systemically administered antibiotic. Int J Pharm 554:284-291.
Wang et al. (2021) Unexpected Function of a Heptapeptide-Conjugated Zwitterionic Polymer that Coassembles into beta-Amyloid Fibrils and Eliminates the Amyloid Cytotoxicity. ACS Appl Mater Interfaces 13(15):18089-18099.
Worch et al. (2019) Stereochemical enhancement of polymer properties. Nature Reviews Chemistry 3(9):514-535.
Yang & Kopecek (2016) Design of smart HPMA copolymer-based nanomedicines. J Control Release 240:9-23.
Yang et al. (2006) Refolding hydrogels self-assembled from N-(2-hydroxypropyl)methacrylamide graft copolymers by antiparallel coiled-coil formation. Biomacromolecules 7(4):1187-1195.
Zamuner et al. (2017) Smart biomaterials: Surfaces functionalized with proteolytically stable osteoblast-adhesive peptides. Bioact Mater 2(3):121-130.
Zhang et al. (2016) C90RF72 poly(GA) aggregates sequester and impair HR23 and nucleocytoplasmic transport proteins. Nat Neurosci 19(5):668-677.
Zhou et al. (2017) Antibodies inhibit transmission and aggregation of C9orf72 poly-GA dipeptide repeat proteins. EMBO Mol Med 9(5):687-702.
Zigmond et al. (2016) Amphiphilic Cross-Linked Liquid Crystalline Fluoropolymer-Poly(ethylene glycol) Coatings for Application in Challenging Conditions: Comparative Study between Different Liquid Crystalline Comonomers and Polymer Architectures. ACS Appl Mater Interfaces 8(49):33386-33393.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A stapled peptide comprising, consisting essentially of, or consisting of two modifications of the amino acid sequence PESKAIKNLLKAVSKERSKRSP (SEQ ID NO: 1) or a truncated version thereof, wherein the two modifications comprise, consist essentially of, or consist of substitutions of two amino acids that are separated by three intervening amino acids with 2-(4-pentenyl) alanines or substitutions of two amino acids that are separated by six intervening amino acids with 2-(4-octenyl) alanine, and further wherein the two amino acids are stapled.

2. The stapled peptide of claim 1, wherein the two modifications are substitutions of amino acids 8 and 12 of SEQ ID NO: 1, or corresponding positions of a modified, optionally truncated, peptide, with 2-(4-pentenyl) alanines and/or the two modifications are substitutions of amino acids 5 and 12 of SEQ ID NO: 1, or corresponding positions of a modified, optionally truncated, peptide, with 2-(4-octenyl) alanines.

3. The stapled peptide of claim 1 or claim 2, wherein the truncated version thereof comprises, consists essentially of, or consists of the amino acid sequence KNLLKAVSKERSKRSP (SEQ ID NO: 2).

4. The stapled peptide of any one of claims 1-3, further comprising a linking moiety at the N-terminus of the stapled peptide, wherein the linking moiety is designed for linking the stapled peptide to a polymer, optionally wherein the linking moiety is an Ac-CGG moiety, a methacrylamide-(CH₂)₆ moiety, or a combination thereof.

5. The stapled peptide of any one of claims 1-4, wherein the stapled peptide is relatively more resistant to protease cleavage than a peptide with the same amino acid sequence that is not stapled, optionally wherein the enhanced protease cleavage resistance results from one or more, optionally all, of the amino acids of the stapled peptide being D-amino acids.

6. A truncated version of the stapled peptide of any one of claims 1-5, optionally wherein the truncated version comprises, consists essentially of, or consists of the amino acid sequence KNLLKAVSKERSKRSP (SEQ ID NO: 2).

7. The stapled peptide of any one of claims 1-5 or the truncated version of claim 6, wherein the substitutions of the two amino acids do not involve any cationic amino acids.

8. A conjugate comprising the stapled peptide of any one of claims 1-5 conjugated to one or more polymers, optionally one or more poly(ethylene glycol) (PEG) molecules, zwitterionic polymers, poly(hydroxypropyl methacrylamide) polymers, or any combination thereof.

9. The conjugate of claim 8, wherein the conjugate is a linear conjugate, a comb-shaped conjugate, a bottlebrush-shaped conjugate, and/or a star-shaped conjugate, optionally wherein the star-shaped conjugate has 3, 4, 5, 6, 7, or 8 arms.

10. The conjugate of claim 8 or claim 9, wherein each arm individually comprises a PEG group of 1-20 kDa.

11. The conjugate of any one of claims 8-10, wherein the conjugate forms micelles of about 10-20 nm diameter in water.

12. A comb-shaped conjugate comprising a stapled peptide of any one of claims 1-5 and/or a truncated stapled peptide of claim 6, wherein at least one stapled peptide is modified with a polymerizable group, optionally a methacrylamide, copolymerized with a hydrophilic vinyl monomer, optionally poly(ethylene glycol) methacrylate, a 2-hydroxypropylmethacrylamide, a zwitterionic methacrylate, or any combination thereof.

13. The comb-shaped conjugate of claim 12, comprising a density of peptide monomers on the comb ranging from 1% to 100%, optionally wherein the comb-shaped conjugate comprises 4, 8, or 16 peptides, further optionally wherein the molecular weight of the comb-shaped conjugate is about 50-70 kDa.

14. A comb-shaped conjugate with a peptide attached at its N- and/or the C-terminus and/or at an intermediate amino acid, optionally wherein the internal amino acid is a lysine comprising an amine-functionalized side chain.

15. The conjugate of any one of claims 8-11 and/or the comb-shaped conjugate of any one of claims 14 and 15, wherein the conjugate and/or the comb-shaped conjugate form a nanoassembly, optionally a micelle, of about 10-20 nm diameter in water.

16. A physical mixture and/or formulation comprising at least one stapled peptide of any one of claims 1-5 and one or more polymers that complex the at least one stapled peptide via an electrostatic interaction, optionally wherein the physical mixture comprises poly(methacrylic acid) and the at least one stapled peptides of any one of claims 1-5.

17. The physical mixture and/or formulation of claim 16, comprising at least one polymer that complexes the at least one stapled peptide via an stereochemistry-driven interaction, optionally wherein the physical mixture comprises a mixture of conjugates of polymers and D-peptide versions of the at least one stapled peptide, wherein a D-peptide version of the at least one stapled peptide is capable of forming a complex with an L-peptide version of the at least one stapled peptide.

18. The physical mixture and/or formulation of claim 17 or claim 18, comprising a D-stapled peptide complexed to one or more conjugates of polymers, L-stapled peptides, or both.

19. A pharmaceutical composition comprising, consisting essentially of, or consisting of the stapled peptide of any one of claims 1-5, the truncated version of claim 6, the conjugate of any one of claims 8-11, the comb-shaped conjugate of any one of claims 14 and 15, or any combination thereof, and a pharmaceutically acceptable carrier, diluent, and/or excipient.

20. The pharmaceutical composition of claim 19, wherein the pharmaceutical composition is pharmaceutically acceptable for use in a human.

21. A medical device comprising a support layer with an antibacterial agent embedded therein or associated therewith, wherein the antibacterial agent comprises the stapled peptide of any one of claims 1-5, the truncated version of claim 6, the conjugate of any one of claims 8-11, the comb-shaped conjugate of any one of claims 14 and 15, or any combination thereof, optionally wherein the medical device is a wound dressing.

22. The medical device of claim 21, wherein the stapled peptide of any one of claims 1-5, the truncated version of claim 6, the conjugate of any one of claims 8-11, the comb-shaped conjugate of any one of claims 14 and 15, or any combination thereof is encapsulated in a particle that is embedded in or associated with the support layer.

23. A method for inhibiting the growth of and/or for killing a bacterium, the method comprising contacting the bacterium with an effective amount of an antibacterial agent selected from the group consisting of the stapled peptide of any one of claims 1-5, the truncated version of claim 6, the conjugate of any one of claims 8-11, the comb-shaped conjugate of any one of claims 14 and 15, or any combination thereof.

24. The method of claim 23, wherein the bacterium is selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to a Salmonella enterica serovars such as but not limited to Salmonella enterica serovar Typhi and Shigella flexneri; and biothreat agents such as but not limited to Bacillus anthracis in both vegetative and spore forms.

25. A method for treating a bacterial infection present in a wound, the method comprising contacting the wound with an effective amount of a composition comprising the stapled peptide of any one of claims 1-5, the truncated version of claim 6, the conjugate of any one of claims 8-11, the comb-shaped conjugate of any one of claims 14 and 15, or any combination thereof.

26. Use of the stapled peptide of any one of claims 1-5, the truncated version of claim 6, the conjugate of any one of claims 8-11, the comb-shaped conjugate of any one of claims 14 and 15, or any combination thereof for preventing or treating a bacterial infection.

27. A composition for use in preventing and/or treating a bacterial infection, the composition comprising, consisting essentially of, or consisting of the stapled peptide of any one of claims 1-5, the truncated version of claim 6, the conjugate of any one of claims 8-11, the comb-shaped conjugate of any one of claims 14 and 15.

28. The composition of claim 27, further comprising a pharmaceutically acceptable carrier, diluent, and/or excipient.

29. The composition of claim 28, wherein the composition is pharmaceutically acceptable for use in a human.
